(19) United States
(12) Patent Application Publication
(10) Pub. No.: US 2007/0275956 A1
(43) Pub. Date: Nov. 29, 2007

(54) Novel Heterocyclic Compounds

(76) Inventors: Braj Bhushan Lohray, Ahemdabad (IN); Vidya Bhushan Lohray, Ahmedabad (IN)

Correspondence Address: NIXON & VANDERHYE, PC, 901 NORTH GLEBE ROAD, 11TH FLOOR, ARLINGTON, VA, 22203, US

(21) Appl. No.: 10/576,130

(22) PCT Filed: Oct. 14, 2004
(86) PCT No.: PCT/IN04/00319

§ 371 Date: May 30, 2007

(30) Foreign Application Priority Data
Oct. 14, 2003 (IN) 1064/MUM/2003

(51) Int. Cl. A61K 31/54; A61K 31/535; A61P 3/00; A61P 9/00; C07D 265/00; C07D 279/22

(52) U.S. Cl. 514/224.8; 514/230.5; 544/105; 544/38

(57) ABSTRACT

Disclosed are novel compounds of formula (I, having hypolipidaemic and hypocholesterolemic properties, their tautomeric forms, their pharmaceutically acceptable salts, their pharmaceutically acceptable solvates and pharmaceutically acceptable compositions containing them.

### FIELD OF INVENTION

The present invention relates to novel hypolipidemic and hypocholesterolemic compounds, their tautomeric forms, their pharmaceutically acceptable salts, their pharmaceutically acceptable solvates and pharmaceutically acceptable compositions containing them. More particularly, the present invention relates to novel heterocycle containing amino acid derivatives of the general formula (I), their tautomeric forms, their pharmaceutically acceptable salts, their pharmaceutically acceptable solvates, pharmaceutical compositions containing them, use of these compounds in medicine and the intermediates involved in their preparation.

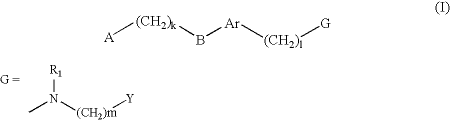

The present invention also relates to a process for the preparation of the above said novel compounds, their tautomeric forms, their pharmaceutically acceptable salts, and pharmaceutical compositions containing them.

The compounds of the general formula (I) lower or modulate triglyceride levels and/or cholesterol levels and/or low-density lipoproteins (LDL) and often raise HDL plasma levels and hence are useful in combating different medical conditions, where such lowering (and raising) is beneficial. Thus, it could be used in the treatment and/or prophylaxis of obesity, hyperlipidaemia, hypercholesteremia, hypertension, and atherosclerotic disease events. These compounds are also useful in the prophylaxis or treatment of diseases where insulin resistance is the underlying pathophysiological mechanism such as impaired glucose tolerance, diabetes, obesity, hyperlipidemia etc.

### BACKGROUND OF THE INVENTION

The present invention discloses compounds suitable for the treatment of hyperlipidemia, diabetes, obesity and similar diseases by modulating the Peroxisome Proliferator Activated Receptor (PPAR). The disease conditions, pathophysiology of the disease conditions, their effects and known & proposed therapies have been described in detail in WO 9119702, WO 9401420, WO 9413650, WO 9503038, WO 9517394, WO 9604260, WO 9604261, WO 9633998, WO 9725042, WO 9736579, WO 9828534, WO 9908501, WO 9916758, WO 9919313, W09920614, WO 0023417, WO 0023445, WO 0023451, WO 0309841, WO 0066572, WO 0116111, WO 0116120, WO 0153257 etc. which are incorporated in their entirety as reference.

Hyperlipidemia has been recognized as the major risk factor in causing cardiovascular diseases due to atherosclerosis [MetS Insights, Sep; 4, 13-17 (2004)]. Atherosclerosis and other such peripheral vascular diseases affect the quality of life of a large population in the world. The therapy aims to lower the elevated plasma LDL cholesterol, low-density lipoprotein and plasma triglycerides in order to prevent or reduce the risk of occurrence of cardiovascular diseases. The detailed etiology of atherosclerosis and coronary artery diseases is discussed by Ross and Glomset [New Engl. J. Med., 295, 369-377 (1976)].

Peroxisome Proliferator Activated Receptor (PPAR) is a member of the steroid/ retinoid/ thyroid hormone receptor family. PPAR∝, PPARγ and PPARδ have been identified as subtypes of PPARS. The role of PPAR, in different disease conditions is widely established PPARγ activation has been found to play a central role in initiating and regulating adipocyte differentiation [Endocrinology 135, 798-800, (1994)] and energy. homeostasis, [Cell, 83, 803-812 (1995); Cell, 99, 239-242 (1999)]. PPARγ agonists would stimulate the terminal differentiation of adipocyte precursors and cause morphological and molecular changes characteristic of a more differentiated, less malignant state. During adipocyte differentiation, several highly specialized proteins are induced, which are being involved in lipid storage and metabolism. It is accepted that PPARγ activation leads to expression of CAP gene [Cell Biology, 95, 14751-14756, (1998)], however, the exact link from PPARγ activation to changes in glucose metabolism and decrease in insulin resistance in muscle has not been clear. PPARα is involved in stimulating β-oxidation of fatty acids [Trends Endocrine. Metabolism, 4, 291-296 (1993)] resulting in plasma circulating free fatty acid reduction [Current Biol., 5, 618-621 (1995)]. The role of PPARs in regulation of obesity-related insulin sensitivity and inflammation [Int J Obes Relat Metab Disord. December; 27 Suppl 3:S17-21(2003)], lipid metabolism and insulin sensitivity [Diabetes February;53 Suppl 1:S43-50 (2004)] have been fairly well established. PPARs are also believed to play a role in diseases associated with metabolic syndrome. [Curr Top Med Chem., 3(14): 1649-61(2003)]. There is growing evidence that PPAR agonists may also influence the cardiovascular system through PPAR receptors as well as directly by modulating vessel wall function [Diabetes Metab., February; 30(1): 7-12 (2004); Drugs Today (Barc), December; 39(12):949-60 (2003)].

PPAR agonists have been found useful in the treatment of obesity [WO 97/36579; Nat Med, April; 10(4):355-61(2004)]. Dual PPAR α and γ agonists have been suggested to be useful for Syndrome X (WO 97/25042). PPAR γ agonists and HMG-CoA reductase inhibitors have exhibited synergism and indicated the usefulness of the combination in the treatment of atherosclerosis and xanthoma [EP 0753 298; Cardiol Rev. May-June; 12(3): 158-70 (2004)].

Leptin is a protein when bound to leptin receptors is involved in sending satiety signal to the hypothalamus. Leptin resistance would therefore lead to excess food in-take, reduced energy expenditure, obesity, impaired glucose tolerance and diabetes [Science, 269, 543-46(1995); Recent Prog Horm Res., 59: 169-205 (2004); Ann N Y Acad Sci, June; 967: 363-78 (2002)]. It has been reported that insulin sensitizers lower plasma leptin concentration [Proc. Natl. Acad. Sci. 93, 5793-5796 (1996); WO 98/02159].

A few compounds belonging to the class of heterocycle containing amino acid derivatives have been reported to be useful in the treatment of hyperlipidemia, hypercholesterolemia, antiobesity and hyperglycemia which includes those described in

i) U.S. Pat. No. 6414002 (Cheng et al.) which discloses compounds of the following general formula

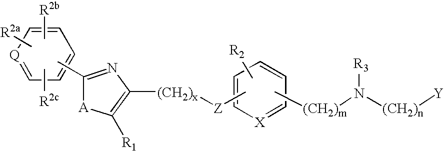

ii) WO 0340114 A1 (Devasthale et al.) which discloses compounds having the following general formula 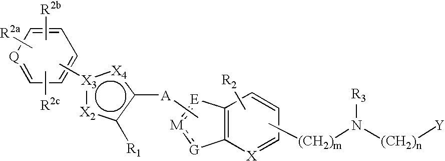
which are incorporated in their entirety as reference. However, none of them have been commercialized so far. There is always an unmet medical need to provide better and cost effective medicines which are better than or of comparable efficacy with the present treatment regimes, and also having a better patient compliant regime.

### SUMMARY OF TIHE INVENTION

The present invention thus provides new compounds of general formula (I) 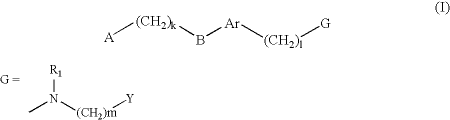
their tautomeric forms, their pharmaceutically acceptable salts, their pharmaceutically acceptable solvates and pharmaceutically acceptable compositions containing them and their use in medicine. The present invention also discloses a process for the preparation of compounds of formula (I) and pharmaceutical compositions containing them.

### OBJECTS OF THE INVENTION

It is an object of this invention is to develop novel compounds represented by the general formula (I) useful for the treatment of hypocholesterolemia, hyperlipidemia, hypoalphalipoproteinemia, obesity, hyperglycemia, hypertriglyceridemia, diabetes mellitus, which may have additional body weight lowering effect and beneficial effect in the treatment and/or prophylaxis of diseases caused by hyperlipidaemia, diseases classified under syndrome X and atherosclerosis.

Another object of the present invention is to provide novel heterocycle containing amino acid derivatives represented by general formula (I), their pharmaceutically acceptable salts, their pharmaceutically acceptable solvates and pharmaceutical compositions containing them or their mixtures thereof and their use in medicine.

Yet another object of this invention is to provide processes for the preparation of novel heterocycle containing amino acid derivatives represented by the general formula (I), their analogs, their tautomeric forms and their pharmaceutically acceptable salts or solvates.

Still another object of the present invention is to provide pharmaceutical compositions containing compounds of the general formula (I), their analogs, their tautomeric forms, their pharmaceutically acceptable salts or their mixtures in combination with suitable carriers, solvents, diluents and other media normally employed in preparing such compositions.

A further object of the present invention is to provide processes for preparation of intermediates involved in the preparation of compounds of formula (I).

### DETAILED DESCRIPTION OF THE INVENTION

Accordingly, the present invention relates to compounds of the general formula (I), 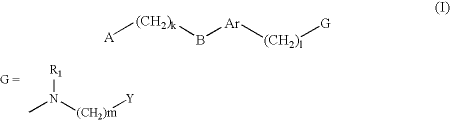
their tautomeric forms, their pharmaceutically acceptable salts, their pharmaceutically acceptable solvates wherein
‘A’ represents an optionally substituted group selected from aryl, heteroaryl, heterocyclyl groups, each of them may optionally be fused, wherein when ‘A’ is substituted, suitable substituents may be selected from hydroxyl, oxo, halo, thio, nitro, amino, cyano, formyl, alkyl, haloalkyl, perhaloalkyl, alkoxy, haloalkoxy, perhaloalkoxy, alkenyl, alkynyl, cycloalkyl, cycloalkenyl, bicycloalkyl, bicycloalkenyl, alkoxy, alkenoxy, cycloalkoxy, aryl, aryloxy, aralkyl, aralkoxy, heterocylyl, heteroaryl, heterocyclylalkyl, heteroaralkyl, heteroaryloxy, heteroaralkoxy, heterocyclyloxy, heterocyclylalkoxy, heterocyclylalkoxyacyl, acyl, acyloxy, acylamino, monosubstituted or disubstituted amino, arylamino, aralkylamino, carboxylic acid and its derivatives such as esters and amides, carbonylamino, hydroxyalkyl, aminoalkyl, alkoxyalkyl, aryloxyalkyl, aralkoxyalkyl, alkylthio, thioalkyl, arylthio, alkylsulfonylamino, alkylsulfonyloxy, alkoxycarbonylamino, aryloxycarbonylamino, aralkyloxycarbonylamind, aminocarbonylamino, alkylaminocarbonylamino, alkoxyamino, hydroxyl amino, sulfenyl derivatives, sulfonyl derivatives, sulfonic acid derivatives;
‘B’ represents oxygen or sulfur; ‘Ar’ represents an optionally substituted divalent aromatic, heteroaromatic or.a heterocyclic group, each of them may optionally be fused, wherein when ‘Ar’ is substituted, suitable substituents may be selected from optionally substituted linear or branched alkyl, alkoxy, .thioalkyl, halogen, haloalkyl, haloalkoxy, acyl, amino, acylamino, thio or carboxylic acid derivatives or sulfonic acids or their derivatives;
R₁ represents hydrogen, optionally substituted groups selected from alkyl (linear or branched), alkenyl (linear or branched), alkynyl (linear or branched), aralkyl, aryloxycarbonyl, alkoxycarbonyl, alkynyloxycarbonyl, alkenyloxycarbonyl, arylcarbonyl, alkylcarbonyl, aryl, heteroaryl, heteroarylcarbonyl, alkylcarbonylamino, arylcarbonylamino, heteroarylcarbonylamino, alkoxycarbonylamino, aryloxycarbonylamino, heteroaryloxycarbonylamino, alkylsulfonyl, alkenylsulfonyl, alkynylsulfonyl, heteroaryloxycarbonyl, heterocyclyloxycarbonyl, alkylaminocarbonyl, arylaminocarbonyl, hydroxyalkyl, alkoxy, alkylsulfonyl, arylthiocarbonyl, heteroarylsulfonyl, arylsulfonyl groups;
when R₁ is substituted, the substituents may be selected from hydroxyl, oxo, halo, thio, nitro, amino, cyano, formyl, alkyl, haloalkyl, perhaloalkyl, alkoxy, haloalkoxy, perhaloalkoxy, alkenyl, alkynyl, cycloalkyl, cycloalkenyl, bicycloalkyl, bicycloalkenyl, alkoxy, alkenoxy, cycloalkoxy, aryl, aryloxy, aralkyl, aralkoxy, heterocylyl, heteroaryl, heterocyclylalkyl, heteroaralkyl, heteroaryloxy, heteroaralkoxy, heterocyclyloxy, heterocyclylalkoxy, heterocyclylalkoxyalkyl, heterocyclylalkoxyacyl, acyl, acyloxy, acylamino, monosubstituted or disubstituted amino, arylamino, aralkylamino, carboxylic acid and its derivatives such as esters and amides; k, l and m are integers independently ranging from 1-3; Y is COR₃ (where R₃ is OH or substituted or unsubstituted alkoxy, aryloxy, aralkyloxy, NH₂, aminoalkyl, amiodialkyl, aminoaralkyl, aminoalkylaralkyl groups);
(CH₂)ₖ, (CH₂)ₗ, (CH₂)ₘ, may be optionally substituted with one or more substituents selected from optionally substituted alkyl, haloalkyl, aryl, alkenyl, alkoxy, aryloxy, aralkoxy, alkoxycarbonyl, aryloxycarbonyl and the like; with the proviso that, ‘A’ does not represent 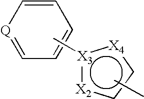
where Q is ‘C’ or ‘N’ and X₂, X₃ & X₄ are independently selected from C, N, 0 or S;

The various groups, radicals and substituents used anywhere in the specification are described in the following paragraphs.

The term “alkyl” used herein, either alone or in combination with other radicals, denotes a linear or branched radical containing one to twelve carbons, such as methyl, ethyl, n-propyl, iso-propyl, n-butyl, sec-butyl, tert-butyl, amyl, t-amyl, in-pentyl, n-hexyl, iso-hexyl, heptyl, octyl and the like.

The term “alkenyl” used herein, either alone or in combination with other radicals, denotes a linear or branched radical containing two to twelve carbons such as vinyl, allyl, 2-butenyl, 3-butenyl 2-pentenyl, 3-pentenyl, 4-pentenyl, 2-hexenyl, 3-hexenyl, 4-hexenyl, 5-hexenyl, 2-heptenyl, 3-heptenyl, 4-heptenyl, 5-heptenyl, 6-heptenyl and the like. The term “alkenyl” includes dienes and trienes of straight and branched chains.

The term “alkynyl” used herein, either alone or in combination with other radicals, denotes a linear or branched radical containing two to twelve carbons, such as ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 3-hexynyl, 4-hexynyl, 5-hexynyl, and the like. The term “alkynyl” includes di- and tri-ynes.

The term “cycloalkyl” used herein, either alone or in combination with other radicals, denotes a radical containing three to seven carbons, such as cyclopropyl, cyclobutyl, cyclopentyl, cyclohexyl, cycloheptyl and the like.

The term “cycloalkenyl” used herein, either alone or in combination with other radicals, denotes a radical containing three to seven carbons, such as cyclopropenyl, 1-cyclobutenyl, 2-cylobutenyl, 1-cyclopentenyl, 2-cyclopentenyl, 3-cyclopentenyl, 1-cyclohexenyl, 2-cyclohexenyl, 3-cyclohexenyl, -cycloheptenyl, cycloheptadienyl, cycloheptatrienyl, and the like.

The term “alkoxy” used herein, either alone or in combination with other radicals; denotes an alkyl radical, as defined above, attached directly to an oxygen atom, such as methoxy, ethoxy, n-propoxy, iso-propoxy, n-butoxy, t-butoxy, iso-butoxy, pentyloxy, hexyloxy, and the like.

The term “alkenoxy” used herein, either alone or in combination with other radicals, denotes an alkenyl radical, as defined above, attached to an oxygen atom, such as vinyloxy, allyloxy, butenoxy, pentenoxy, hexenoxy, and the like.

The term “cycloalkoxy” used herein, either alone or in combination with other radicals, denotes a cycloalkyl radical as defined above, attached directly to an oxygen atom, such as cyclopropyloxy, cyclobutyloxy, cyclopentyloxy, cyclohexyloxy, cycloheptyloxy and the like.

The term “halo” or “halogen” used herein, either alone or in combination with other radicals, such as “haloalkyl”, “perhaloalkyl” etc refers to a fluoro, chloro, bromo or iodo group. The term “haloalkyl” denotes an alkyl radical, as defined above, substituted with one or more halogens such as perhaloalkyl, preferably, perfluoro(C₁-C₆)alkyl such as fluoromethyl, difluoromethyl, trifluoromethyl, fluoroethyl, difluoroethyl, trifluoroethyl, mono or polyhalo substituted methyl, ethyl, propyl, butyl, pentyl or hexyl groups. The term “haloalkoxy” denotes a haloalkyl, as defined above, directly attached to an oxygen atom, such as fluoromethoxy., chloromethoxy, fluoroethoxy chloroethoxy groups, and the like. The term “perhaloalkoxy” denotes a perhaloalkyl radical, as defined above, directly attached to an oxygen atom, trifluoromethoxy, trifluoroethoxy, and the like.

The term “aryl” or “aromatic” used herein, either alone or in combination with other radicals, denotes an aromatic system containing one, two or three rings wherein such rings may be attached together in a pendant manner or may be fused, such as phenyl, naphthyl, tetrahydronaphthyl, indane, biphenyl, and the like. The term ‘aralkyl” denotes an alkyl group, as defined above, attached to an aryl, such as benzyl phenethyl, naphthylmethyl, and the like. The term “aryloxy” denotes an aryl radical, as defined above, attached to an alkoxy group, such as phenoxy, naphthyloxy and the like, which may be substituted. The term “aralkoxy” denotes an arylalkyl moiety, as defined above, such as benzyloxy, phenethyloxy, naphthylmethyloxy, phenylpropyloxy, and the like, which may be substituted.

The term “heterocyclyl” or “heterocyclic” used herein, either alone or in combination with other radicals, denotes saturated, partially saturated and unsaturated ring-shaped radicals containing one or more hetero atoms selected from nitrogen, sulfur and oxygen. Examples of saturated heterocyclic radicals include aziridinyl, azbtidinyl, pyrrolidinyl, imidazolidinyl, piperidinyl, piperazinyl, 2-oxopiperidinyl, 4-oxopiperidinyl, 2-oxopiperazinyl, 3-oxopiperazinyl, morpholinyl, thiomorpholinyl, 2-oxomorpholinyl, azepinyl, diazepinyl, oxapinyl, thiazepinyl, oxazolidinyl, thiazolidinyl, and the like; examples of partially saturated heterocyclic radicals include dihydrothiophene, dihydropyran, dihydrofuran, dihydrothiazole, and the like.

The term “heteroaryl” or “heteroaromatic” used herein, either alone or in combination with other radicals, denotes aromatic radicals containing one or more hetero atoms selected from O, N, S or unsaturated 5 to 6 membered heterocyclic radicals containing one or more hetero atoms selected from O, N or S, attached to an aryl group, such as pyridyl, thienyl, firyl, pyrrolyl, oxazolyl, thiazolyl, isothiazolyl, imidazolyl, isoxazolyl, oxadiazolyl, thiadiazolyl, triazolyl, tetrazolyl, benzopyranyl, benzopyranonyl, benzofuranyl, benzothienyl, indolinyl, indolyl, quinolinyl, pyrimidinyl, pyrazolyl, quinazolinyl, quinazolonyl, pyrimidonyl, pyridazinyl, triazinyl, benzoxazinyl, benzoxazinonyl, benzothiazinyl, benzothiazinonyl, benzoxazolyl, benzothiazolyl, benzimidazolyl, benzotriazolyl, phthalazynil, naphthylidinyl, purinyl, carbazolyl, phenothiazinyl, phenoxazinyl, and the like.

The term “heterocyclylalkyl” used herein, either alone or in combination with other radicals, represents a heterocyclyl group, as defined above, substituted with an alkyl group of one to twelve carbons, such as pyrrolidinealkyl, piperidinealkyl, morpholinealkyl, thiomorpholinealkyl, oxazolinealkyl, and the like, which may be substituted. The term “heteroaralkyl” used herein, either alone or in combination with other radicals, denotes a heteroaryl group, as defined above, attached to a straight or branched saturated carbon chain containing 1 to 6 carbons, such as (2-furyl)methyl, (3-furyl)methyl, (2-thienyl)methyl, (3-thienyl)methyl, (2-pyridyl)methyl, 1-methyl-1-(2-pyrimidyl)ethyl and the like. The terms “heteroaryloxy”, “heteroaralkoxy”, “heterocycloxy”, “heterocylylalkoxy” denotes heteroaryl, heteroarylalkyl, heterocyclyl, heterocylylalkyl groups respectively, as defined above, attached to an oxygen atom.

The term “acyl” used herein, either alone or in combination with other radicals, denotes a radical containing one to eight carbons such as formyl, acetyl, propanoyl, butanoyl, iso-butanoyl, pentanoyl, hexanoyl, heptanoyl, benzoyl and the like, which may be substituted.

The term “acyloxy” used herein, either alone or in combination with other radicals, denotes a radical acyl, as defined above, directly attached to an oxygen atom, such as acetyloxy, propionyloxy, butanoyloxy, iso-butanoyloxy, benzoyloxy and the like.

The term “acylamino” used herein, either alone or in combination with other radicals, denotes an acyl group as defined earlier, may be CH₃CONH, C₂H₅CONH, C₃H₇CONH, C₄H₉CONH, C₆H₅CONH and the like, which may be substituted.

The term “mono-substituted amino” used herein, either alone or in combination with other radicals, denotes an amino group, substituted with one group selected from (C₁-C₆)alkyl, substituted alkyl, aryl, substituted aryl or arylalkyl groups. Examples of monoalkylamino group include methylamine, ethylamine, n-propylamine, n-butylamine, n-pentylamine and the like.

The term ‘disubstituted amino” used herein, either alone or in combination with other radicals, denotes an amino group, substituted with two radicals that may be same or different selected from (C₁-C₆)alkyl, substituted alkyl, aryl, substituted aryl, or arylalkyl groups, such as dimethylamino, methylethylamino, diethylamino, phenylmethyl amino and the like.

The term “arylamino” used herein, either alone or in combination with other radicals, denotes an aryl group, as defined above, linked through amino having a free valence bond from the nitrogen atom, such as phenylamino, naphthylamino, N-methyl anilino and the like.

The term “aralkylamino” used herein, either alone or in combination with other radicals, denotes an arylalkyl group as defined above linked through amino having a free valence bond from the nitrogen atom e.g. benzylamino, phenethylamino, 3-phenylpropylamino, 1-napthylmethylamino, 2-(1-napthyl)ethylamino and the like.

The term “oxo” or “carbonyl” used herein, either alone (—C═O—) or in combination with other radicals, such as “alkylcarbonyl”, “alkoxycarbonyl”, “arylcarbonyl”, “aryloxycarbonyl”, “heteroarylcarbonyl”, “heteroaryloxycarbonyll”, “heterocyclyloxycarbonyl” denotes a carbonyl radical (—C═O—) substituted with an alkyl, alkoxy, aryl, aryloxy, heteroaryl; heteroaryloxy, heterocyclyloxy radicals as described above.

The term “carboxylic acid” used herein, alone or in combination with other radicals, denotes a —COOH group, and includes derivatives of carboxylic acid such as esters and amides. The term “ester” used herein, alone or in combination with other radicals, denotes —COO— group, and includes carboxylic acid derivatives, where the ester moieties are alkoxycarbonyl, such as methoxycarbonyl, ethoxycarbonyl, and the like, which may be substituted; aryloxycarbonyl group such as phenoxycarbonyl, napthyloxycarbonyl, and the like, which may be substituted; aralkoxycarbonyl group such as benzyloxycarbonyl, phenethyloxycarbonyl, napthylmethoxycarbonyl, and the like, which may be substituted; heteroaryloxycarbonyl, heteroaralkoxycarbonyl, wherein the heteroaryl group, is as defined above, which may be substituted; heterocyclyloxycarbonyl, where the heterocyclic group, as defined earlier, which may be substituted.

The term “amide” used herein, alone or in combination with other radicals, represents an aminocarbonyl radical (H₂N—C═O—), wherein the amino group is mono- or di-substituted or unsubstituted, such as methylamide, dimethylamide, ethylamide, diethylamide, and the like. The term “aminocarbonyl” used herein, either alone or in combination with other radicals, with other terms such as ‘aminocarbonylalkyl”, “n-alkylaminocarbonyl”, “N-arylaminocarbonyl”, “N,N-dialkylaminocarbonyl”, “N-alkyl-N-arylaminocarbonyl”, “N-alkyl-N-hydroxyaminocarbonyl”, and “N-alkyl-N-hydroxyaminocarbonylalkyl”, substituted or unsubstituted. The terms “N-alkylaminocabonyl” and “N,N-diallylaminocarbonyl” denotes aminocarbonyl radicals, as defined above, which have been substituted with one alkyl radical and with two alkyl radicals, respectively. Preferred are “lower alkylaminocarbonyl” having lower alkyl radicals as described above attached to aminocarbonyl radical. The terms “N-arylaminocarbonyl” and “N-alkyl-N-arylaminocarbonyl” denote amiocarbonyl radicals substituted, respectively, with one aryl radical, or one alkyl, and one aryl radical. The term “aminocarbonylalkyl” includes alkyl radicals substituted with aminocarbonyl radicals.

The term “hydroxyalkyl” used herein, either alone or in combination with other radicals, denotes an alkyl group, as defined above, substituted with one or more hydroxy radicals, such as hydroxymethyl, hydroxyethyl, hydroxypropyl, hydroxybutyl, hydroxypentyl, hydroxyhexyl and the like.

The term “aminoalkyl” used herein, alone or in combination with other radicals, denotes an amino (—NH₂) moiety attached to an alkyl radical, as defined above, which may be substituted, such as mono- and di-substituted aminoalkyl. The term “alkylamino” used herein, alone or in combination with other radicals, denotes an alkyl radical, as defined above, attached to an amino group, which may be substituted, such as mono- and di-substituted alkylamino.

The term “alkoxyalkyl” used herein, alone or in combination with other radicals, denotes an alkoxy group, as defined above, attached to an alkyl group, such as methoxymethyl, ethoxymethyl, methoxyethyl, ethoxyethyl and the like. The term “aryloxyalkyl” used herein, alone or in combination with other radicals, includes phenoxymethyl, napthyloxymethyl, and the like. The term “aralkoxyalkyl” used herein, alone or in combination with other radicals, includes C₆H₅CH₂OCH₂, C₆H₅CH₂OCH₂CH₂, and the like.

The term “alkylthio” used herein, either alone or in combination with other radicals, denotes a straight or branched or cyclic monovalent substituent comprising an alkyl group of one to twelve carbon atoms, as defined above, linked through a divalent sulfur atom having a free valence bond from the sulfur atom, such as methylthio, ethylthio, propylthio, butylthio, pentylthio and the like. Examples of cyclic alkylthio are cyclopropylthio, cyclobutylthio, cyclopentylthio, cyclohexylthio and the like, which may be substituted.

The term “thioalkyl” used herein, either alone or in combination with other radicals, denotes an alkyl group, as defined above, attached to a group of formula —SR′, where R′ represents hydrogen, alkyl or aryl group, e.g. thiomethyl, methylthiomethyl, phenylthiomethyl and the like, which may be substituted.

The term “arylthio” used herein, either alone or in combination with other radicals, denotes an aryl group, as defined above, linked through a divalent sulfur atom, having a free valence bond from the sulfur atom such as phenylthio, napthylthio and the like.

The term “alkoxycarbonylamino” used herein, alone or in combination with other radicals, denotes an alkoxycarbonyl group, as defined above, attached to an amino group, such as methoxycarbonylamino, ethoxycarbonylamino, and the like. The term “aryloxycarbonylamino” used herein, alone or in combination with other radicals, denotes an aryloxycarbonyl group, as defined above, attached to the an amino group, such as C₆H₅OCONH, C₆H₅OCONCH₃, C₆H₅OCONC₂H₅, C₆H₄(CH₃O)CONH, C₆H₄(OCH₃)OCONH, and the like. The term “aralkoxycarbonylamino” used herein, alone or in combination with other radicals, denotes an aralkoxycarbonyl group, as defined above, attached to an amino group C₆H₅CH₂OCONH, C₆H₅CH₂CH₂CH₂OCONH, C₆H₅CH₂OCONHCH₃, C₆H₅CH₂OCONC₂H₅, C₆H₄(CH₃)CH₂OCONH, C₆H₄(OCH₃)CH₂OCONH, and the like.

The term “aminocarbonylamino”, “alkylaminocarbonylamino”, “dialkylaminocarbonylamino” used herein, alone or in combination with other radicals, denotes a carbonylamino (—CONH₂) group, attached to amino(NH₂), alkylamino group or dialkylamino group respectively, where alkyl group is as defined above.

The term “alkoxyamino” used herein, alone or in combination with other radicals, denotes an alkoxy group, as defined above, attached to an amino group. The term “hydroxyamino” used herein, alone or in combination with other radicals, denotes —NHOH moiety, and may be substituted.

The term “sulfenyl” or “sulfenyl and its derivatives” used herein, alone or in combination with other radicals, denotes a bivalent group, —SO— or RₙSO, where Rₙ is substituted or unsubstituted alkyl, aryl, heteroaryl, heterocyclyl, and the like.

The term “sulfonyl” or “sulfones and its derivatives” used herein, either alone or in combination with other radicals, with other terms such as alkylsulfonyl, denotes divalent radical —SO₂—, or RₙSO₂—, where Rₙ is substituted or unsubstituted groups selected from alkyl, aryl, heteroalyl, heterocyclyl, and the like. “Alkylsulfonyl” denotes alkyl radicals, as defined above, attached to a sulfonyl radical, such as methylsulfonyl, ethylsulfonyl, propylsulfonyl and the like. The term “arylsulfonyl” used herein, either alone or in combination with other radicals, denotes aryl radicals, as defined above, attached to a sulfonyl radical, such as phenylsulfonyl and the like.

The term “substituted” used in combination with other radicals including when used as substitutions on any of the substituents, denotes suitable substituents on that radical such as substituted alkyl, substituted alkenyl, substituted alkynyl, substituted cycloalkyl, substituted aryl, etc, mentioned anywhere in the specification. The suitable substituents include, but are not limited to the following radicals, alone or in combination with other radicals- hydroxyl, oxo, halo, thio, nitro, amino, cyano, formyl, alkyl, haloalkyl, perhaloalkyl, alkoxy, haloalkoxy, perhaloalkoxy, alkenyl, alkynyl, cycloalkyl, cycloalkenyl, bicycloalkyl, bicycloalkenyl, alkoxy, alkenoxy, cycloalkoxy, aryl, aryloxy, aralkyl, aralkoxy, heterocylyl, heteroaryl, heterocyclylalkyl, heteroaralkyl, heteroaryloxy, heteroaralkoxy, heterocyclyloxy, heterocyclylalkoxy, heterocyclylalkoxyalkyl, heterocyclylalkoxyacyl, acyl, acyloxy, acylamino, monosubstituted or disubstituted amino, arylamino, aralkylamino, carboxylic acid and its derivatives such as esters and amides, carbonylamino, hydroxyalkyl, aminoalkyl, alkoxyalkyl, aryloxyalkyl, aralkoxyalkyl, alkylthio, thioalkyl, arylthio, alkoxycarbonylamino, aryloxycarbonylamino, aralkoxycarbonylamino, aralkyloxycarbonylamino, aminocarbonylamino, alkylaminocarbonylamino, dialkylaminocarbonylamino, alkoxyamino, hydroxyl amino, sulfenyl derivatives, sulfonyl derivatives.

Suitable groups and substituents on the groups may be selected from those described anywhere in the specification.

Particularly useful compounds according to the present invention includes Ethyl-[4-(2-phenoxazin-10-yl-ethoxy)-benzylamino]-acetate; Ethyl-[4-(2-phenothiazin-10-yl-ethoxy)-benzylamino]-acetate; Methyl-[4-(2-oxo-3-phenyl-oxazolidin-5-ylmethoxy)-benzylamino]-acetate; Ethyl-[(6-benzyloxy-naphthalen-2-ylmethyl)-axmno]-acetate; Ethyl-([6-(1-phenyl-pentyloxy)-naphthalen-2-ylmethyl]-amino }-acetate; Ethyl-[4-(2-carbazol-9-yl-ethoxy)-benzyiamino]-acetate; Ethyl-[4-(1-pyridin-2-yl-pyrrolidin-2-ylmethoxy)-benzylamino]-acetate; Ethyl-{4-[2-(2,3-dihydro-benzo[1,4]oxazin-4-yl)-ethoxy]-benzylamino}-acetate; Ethyl-(benzyl-{3-[2-(3,4-dihydro-2H-quiolin-1-yl)-ethoxy]-benzyl} -amino)-acetate; Ethyl-(benzyl-{3-[2-(4-methanesulfonyloxy-phenyl)-ethoxy]-benzyl)-amino)-acetate; Ethyl-(benzyl-{3-[2-(4-hydroxy-phenyl)-ethoxyl-benzyl}-amino)-acetate; Ethyl-(benzyl-[3-(2-phenoxazin-10-yl-ethoxy)-benzyl]-amino}-acetate; Ethyl-{benzyl-[3-(2-carbazol-9-yl-ethoxy)-benzyl]-ammo}-acetate; Ethyl-(benzyl-{3-[2-(5-ethyl-pyridin-2-yl)-ethoxy]-benzyl}-amino)-acetate; Ethyl-(benzyl-{3-[2-(2,3-dihydro-benzo[1,4]pxazin-4-yl)-ethoxy]-benzyl}-amino)-acetate; Ethyl (benzyl-{3-[2-(2,3-dihydo-benzo[1,4]thiazin-4-yl)-ethoxy]-benzyl}-amino)-acetate, Ethyl-{benzyl-[3-(2-indol-1-yl-ethoxy)-benzyll-amiino }-acetate; Ethyl-{benzyl-[3-(3-phenothiazin-10-yl-propoxy)-benzyl]-amino}-acetate; Ethyl-{benzyl-[3-(3-methyl-4-oxo-3,4-dihydro-quinazolin-2-ylmethoxy)-benzyl]-amino) - acetate; Ethyl-[benzyl-(3-{2-[2-methyl-5-(4-methylsulfanyl-phenyl)-pyrrol-1-yl]-ethoxy)-benzyl)-amino]-acetate; Ethyl-{benzyl-[3-(2-oxo-3-phenyi-oxazohdin-5-ylmethoxy)-benzyl]-amino}-acetate; Ethyl-{(4-methoxy-phenoxycarbonyl)-[4-(2-phenoxazin-10-yl-ethoxy)-benzyl]-amino)- acetate; Ethyl-{(4-methoxy-phenoxycarbonyl)-[4-(2-phenothiazin- 10-yl-ethoxy)-benzyl]-amino}-acetate; Methyl-{(4-methoxy-phenoxycarbonyl)-[4-(2-oxo-3-phenyl-oxazolidin-5-ylmethoxy)-benzyl]-amino }-acetate; Ethyl-[(6-benzyloxy-naphthalen-2-ylmethyl)-(4-methoxy-phenoxycarbonyl)-amino]-acetate; Methyl-{(4-methoxy-phenoxycarbonyl)-[6-(1-phenyl-pentyloxy)-naphthalen-2-ylmethyl]-amino}-acetate; Ethyl-[{4-[2-(6,7-dihydro-4H-thieno[3,2-c]pyridin-5-yi)-ethoxy]-benzyl} -(4-methoxy- phenoxycarbonyl)-amino ]-acetate; Ethyl-[{4-[2-(2,3-dihydro-benzo[1,4]thiazin-4-yl)-ethoxy]-benzyl}-(4-methoxy- phenoxycarbonyl)-amino]-acetate; Ethyl-[[4-(2-indol-1-yl-ethoxy)-benzyl]-(4-methoxy-phenoxycarbonyl)-amino]-acetate; Ethyl-[[4(2-carbazol-9-yi-ethoxy)-benl]-(4-met hoxy-phenoxycarbonyl)-anino]-acetate; Ethyl-{(4-methoxy-phenoxycarbonyl)-[4-(1-pyridin-2-yl-pyrrolidin-2-ylmethoxy)-benzyl]- amino)}-acetate; Ethyl-[{4-[2-(2,3-dihydro-benzo[1,4]oxazin-4-yl)-ethoxy]-benzyl}-(4-methoxy- phenoxycarbonyl)-amino]-acetate; Ethyl-(ethoxycarbonylmethyl-{4-[2-(5-methyl-2-phenyl-oxazol-4-yl)-ethoxy]-benzyl} - amino)-acetate; Ethyl-(benzyl-{4-[2-(5-methyl-2-thiophen-2-yl-oxazol-4-yl)-ethoxy]-benzyl}-amino)- acetate; Ethyl-(benzyl-(3-[2-(5-methyl-2-thiophen-2-yl-oxazol4-yl)-ethoxy]-benzyl}-amino)- acetate; Ethyl-[benzyl-(4-{2-[2-methyl-5-(4-methylsulfanyl-phenyl)-pyrrol-1-yl]-ethoxy}-benzyl)- amino]-acetate; Ethyl-(benzyl-{4-[2-(methyl-pyridin-2-yl-amino)-ethoxy]-benzyl}-amino)-acetate; Ethyl-(benzyl-{4-[2-(5-ethyl-pyridin-2-yl)-ethoxy]-benzyl)-amino)-acetate; Ethyl-{benzyl-[4-(2-fluoro-benzyloxy)-benzyl]-amino}-acetate; Ethyl-[benzyl-(4-{2-[2-(4-methoxy-phenyl)-5-methyl-pyrrol-1-yl]-ethoxy)-benzyl)-amino]-acetate; Ethyl-[benzyl-(4-{2-[2-methyl-5-(5-methyl-thiophen-2-yl)-pyrrol-1-yl]-ethoxy}-benzyl)- amino]-acetate; Ethyl-[benzyl-(4-{2-[5-methyl-2-(5-methyl-thiophen-2-yl)-oxazol-4-yl]-ethoxy}-benzyl)- amino]-acetate; Ethyl-(benzyl-{4-[2-(2,3-dihydro-benzo[1,4]thiazin4-yl)-ethoxy]-benzyl}-amino)-acetate; Ethyl-(benzyl-{4-[2-(5-methyl-2-thiophen-3-yl-oxazol-4-yl)-ethoxy]-benzyl}-amino)-acetate; Ethyl-({4-[2-(2-benzo[b]thiophen-2-yl-5-methyl-oxazol-4-yl)-ethoxy]-benzyl)-benzy1-amino)-acetate; Ethyl-(benzyl-[4-(3-methyl-4-oxo-3,4-dihydro-quinazolin-2-ylmethoxy)-benzyl]-amino)-acetate; Ethyl-(benzyl-[4-(2-phenoxazin-10-yl-ethoxy)-benzyl]-amino}-acetate; Ethyl-(benzyl-{4-[2-(3,4-dihydro-2H-quinolin-1-yl)-ethoxy]-benzyl}-amino)-acetate; Ethyl-[{4-[2-(5-ethyl-pyridin-2-yl)-ethoxy]-benzyl}-(4-methoxy-phenoxycarbonyl)-amino]-acetate; Ethyl-((4-methoxy-phenoxycarbonyl)--4-[2-(methyl-pyridin-2-yl-amino)-ethoxy]-benzyl)- amino)-acetate; Ethyl-[(4-methoxy-phenoxycarbonyl)-(4-(2-[2-methyl-5-(5-methyl-thiophen-2-yl)-pyrrol-1-yl]-ethoxy)-benzyl)-amino]-acetate; Methyl-((4-methoxy-phenoxycarbonyl)-{4-[2-(5-methyl-2-thiophen-2-yl-oxazol-4-yl)-ethoxy]-benzyl}-amino)-acetate; Ethyl-[(4-methoxy-phenoxycarbonyl)-(4-{2-[5-methyl-2-(5-methyl-thiophen-2-yl)-oxazol-4-yl]-ethoxy}-benzyl)-amino]-acetate; Ethyl-{(4-methoxy-phenoxycarbonyl)-[4-(3-methyl-4-oxo-3,4-dihydro-quinazolin-2- ylmethoxy)-benzyl]-amino}-acetate; Ethyl-[[4-(2-fluoro-benzyloxy)-benzyl]-(4-methoxy-phenoxycarbonyl)-amino]-acetate; Ethyl-[(4-methoxy-phenoxycarbonyl)-(4-{2-[2-methyl-5-(4-methylsulfanyl-phenyl)- pyrrol- 1-yl]-ethoxy}-benzyl)-amino]-acetate; Ethyl-[{4-[2-(2-furan-2-yl-5-methyl-oxazol-4-yl)-ethoxy]-benzyl) -(4-methoxy- phenoxycarbonyl)-amino]-acetate; Ethyl-[(4-[2-(3-ethyl-4-methyl-6-oxo-2-thioxo-3,6-dihydro-2H-pyrimidin-1-yl)-ethoxy]-benzyl}-(4-methoxy-phenoxycarbonyl)-amino]-acetate; Ethyl-((4-methoxy-phenoxycarbonyl)-{4-[2-(2,5,6-trimethyl-4-oxo-4H-thieno[2,3-d]pyrimidin-3-yl)-ethoxy]-benzyl}-amino)-acetate; Ethyl-[(4-[2-(3,4-dihydro-2H-quinolin- 1-yl)-ethoxy]-benzyl}-(4-methoxy- phenoxycarbonyl)-amino]-acetate; Ethyl-[{3-[2-(2-furan-2-yl-5-methyl-oxazol4-yl)-ethoxy]-benzyl}-(4-methoxy- phenoxycarbonyl)-amino]-acetate; Ethyl-((4-methoxy-phenoxycarbonyl)-(4-[2-(methyl-pyrimidin-2-yl-amino)-ethoxy]- benzyl}-amino)-aacetate; Ethyl-[{4-[2-(2-benzo[1,3]dioxol-5-yl-5-methyl-pyrrol-1-yl)-ethoxy]-benzyl)-(4-methoxy-phenoxycarbonyl)-amino]-acetate; Ethyl-[[4-(2-benzoimidazol-1-yl-ethoxy)-benzyl]-(4-methoxy-phenoxycarbonyl)-amino]- acetate; Ethyl-{3-[2-(2-benzo[1,3]dioxol-5-yl-5-methyl-pyrrol-1-yl)-ethoxy]-benzyl}-(4-methoxy- phenoxycarbonyl)-amino]-acetate; Ethyl-[[4-(2-benzoimidazol- 1-yl-ethoxy)-benzyl]-(4-methoxy-phenoxycarbonyl)-amino]- acetate; Ethyl-{(4-methoxy-phenoxycarbonyl)-[4-(1-methyl-1H-benzoimidazol-2-ylmethoxy)- benzyl]-amino}-acetate; Ethyl-[(4-methoxy-phenoxycarbonyl)-(4-{2-[5-methyl-2-(5-methyl-furan-2-yl)-oxazol-4-yl]-ethoxy}-benzyl)-amino]-acetate; Ethyl-[[4-(6-methoxy-1-methyl-1H-benzoimidazol-2-ylmethoxy)-benzyl]-(4- ethoxy- phenoxycarbonyl)-amino]-acetate; Ethyl-[(4-{2-[2-(5-bromo-thiophen-2-yl)--methyl-oxazol-4-yl]-ethoxy} -benzyl)-(4- methoxy-phenoxycarbonyl)-amino]-acetate; Ethyl-[[4-(benzothiazol-2-ylmethoxy)-benzyl]-(4-methoxy-phenoxycarbonyl)-amino]- acetate; Ethyl-[benzyloxycarbonyl-(4-(2-[2-methyl-5-(5-methyl-thiophen-2-yl)-pyrro1-1-yl]- ethoxy}-benzyl)-amino]-acetate; Ethyl- ((4-methoxy-phenoxycarbonyl)-[4-(2-morpholin-4-yl-ethoxy)-benzyl]-amino}- acetate; Ethyl-[(4-methoxy-phenoxycarbonyl)-(4-{2-[methyl-(4-nitro-phenyl)-amino]-ethoxy)- benzyl)-amino]-acetate; Ethyl-[(4-methoxy-phenoxycarbonyl)-(3-(2-[2-methyl-5-(5-methyl-thiophen-2-yl)-pyrrol- 1-yl]ethoxy}benzyl)-amino]-acetate; Ethyl-[[4-(benzooxazol-2-ytmethoxy)-benzyl]-(4-methoxy-phenoxycarbonyl)-amino]- acetate; Ethyl-[(4-methoxy-phenoxycarbonyl)-(3-{2-[2-methyl-5-(4-methylsulfanyl-phenyl)- pyrrol- 1-yl]-ethoxy}-benzyl)-amino]-acetate; Ethyl-((4-methoxy-phenoxycarbonyl)-{4-[2-(5-methyl-3-phenyl-isoxazol4-yl)-ethoxy]- benzyl) -amino)-acetate; (13enzyl-{3-[2-(3,4-dihydro-2H-quinolin-1-yl)-ethoxy]-benzyl}-amino)-acetic acid and its pharmaceutically acceptable salts; ([3enzyl-{3-[2-(4-methanesulfonyloxy-phenyl)-ethoxy]-benzyl}-amino)-acetic acid and its pharmaceutically acceptable salts; {Benzyl-[3-(2-phenoxazin-10-yl-ethoxy)-benzyl]-amino}-acetic acid and its pharmaceutically acceptable salts; {Benzyl-[3-(2-carbazol-9-yl-ethoxy)-benzyl]-amino}-acetic acid and its pharmaceutically acceptable salts; (3enzyl-{3-[2-(5-ethyl-pyridin-2-yl)-ethoxy]-benzyl}-amino)-acetic acid and its pharmaceutically acceptable salts; (Benzyl-{3-[2-(2,3-dihydro-benzo[1,4]oxazin4-yl)-ethoxy]-benzyl}-amino)-acetic acid and its pharmaceutically acceptable salts; (Benzyl-{3-[2-(2,3-dihydro-benzo[1,4]thiazin-4-yl)-ethoxy]-benzyl}-amino)-acetic acid and its pharmaceutically acceptable salts; {Benzyl-[3-(2-indol-1-yl-ethoxy)-benzyl]-amino}-acetic acid and its pharmaceutically acceptable salts; {Benzyl-[3-(3-phenothiazin-10-yl-propoxy)-benzyl]-amino}-acetic acid and its pharmaceutically acceptable salts; {Benzyl-[3-(3-methyl4-oxo-3,4-dihydro-quinazolin-2-ylmethoxy)-benzyl]-amino} -acetic acid and its pharmaceutically acceptable salts; [Benzyl-(3-{2-[2-methyl-5-(4-methylsulfanyl-phenyl)-pyrrol-1-yl]-ethoxy}-benzyl)- amino]-acetic acid and its pharmaceutically acceptable salts; {(4-Methoxy-phenoxycarbonyl)-[4-(2-phenoxazin-10-yl-ethoxy)-benzyl]-amino}-acetic acid and its pharmaceutically acceptable salts; {(4-Methoxy-phenoxycarbonyl)-[4-(2-phenothiazin-10-yl-ethoxy)-benzyl]-amino}-acetic acid and its pharmaceutically acceptable salts; {(4-Methoxy-phenoxycarbonyl)-[4-(2-oxo-3-phenyl-oxazolidin-5-ylmethoxy)-benzyl]- amino}-acetic acid and its pharmaceutically acceptable salts; [(6-Benzyloxy-naphthalen-2-ylmethyl)-(4-methoxy-phenoxycarbonyl)-amino]-acetic acid and its pharmaceutically acceptable salts; {(4-Methoxy-phenoxycarbonyl)-[6-(1-phenyl-pentyloxy)-naphthalen-2-ylmethyl]-amino}- acetic acid and its pharmaceutically acceptable salts; [{4-[2-(6,7-Dihydro-4H-thieno[3,2-c]pyridin-5-yl)-ethoxy]-benzyl}-(4-methoxy- phenoxycarbonyl)-amino]-acetic acid and its pharmaceutically acceptable salts; [{4-[2-(2,3-Dihydro-benzo[1,4]thiazin-4-yl)-ethoxy]-benzyl}-(4-methoxy- phenoxycarbonyl)-amino]-acetic acid and its pharmaceutically acceptable salts; [[4-(2-Indol-1-yl-ethoxy)-benzyl]-(4-methoxy-phenoxycarbonyl)-amino]-acetic acid and its pharmaceutically acceptable salts; [[4-(2-Carbazol-9-yl-ethoxy)-benzyl]-(4-methoxy-phenoxycarbonyl)-amino]-acetic acid and its pharmaceutically acceptable salts; {(4-Methoxy-phenoxycarbonyl)-[4-(1-pyridin-2-yl-pyrrolidin-2-ylmethoxy)-benzyl]- amino}-acetic acid and its pharmaceutically acceptable salts; [{4-[2-(2,3-Dihydro-benzo[1,4]oxazin-4-yl)-ethoxy]-benzyl)-(4-methoxy- phenoxycarbonyl)-amino]-aceticacid and its pharmaceutically acceptable salts; (Carboxymethyl-{4-[2-(5-me.thyl-2-phenyl-oxazol-4-yl)-ethoxy]-benzyl}-amino)-acetic acid and its pharmaceutically acceptable salts; (Benzyl-{4-[2-(5-methyl-2-thiophen-2-yl-oxazol-4-yl)-ethoxy]-benzyl} -amino)-acetic acid and its pharmaceutically acceptable salts; (Benzyl-{3-[2-(5-methyl-2-thiophen-2-yl-oxazol-4-yl)-ethoxy]-benzyl}-amino)-acetic acid and its pharmaceutically acceptable salts; [Benzyl-(4-{2-[2-methyl-5-(4-methylsulfanyl-phenyl)-pyrrol-1-yl]-ethoxy}-benzyl)- amino]-acetic acid and its pharmaceutically acceptable salts; (Benzyl-{4-[2-(methyl-pyridin-2-yl-amino)-ethoxy]-benzyl)-amino)-acetic acid and its pharmaceutically acceptable salts; (Benzyl-{4-[2-(5-ethyl-pyridin-2-yl)-ethoxy]-benzyl}-amino)-acetic acid and its pharmaceutically acceptable salts; {Benzyl-[4-(2-fluoro-benzyloxy)-benzyl]-amino}-acetic acid and its pharmaceutically acceptable salts; [Benzyl-(4-{2-[2-(4-methoxy-phenyl)-5-methyl-pyrrol- 1-yl]-ethoxy}-benzyl)-amino]- acetic acid and its pharmaceutically acceptable salts; [Benzyl-(4-{2-[2-methyl-5-(5-methyl-thiophen-2-yl)-pyrrol-1-yl]-ethoxy}-benzyl)-amino]- acetic acid and its pharmaceutically acceptable salts; [Benzyl-(4-{2-[5-methyl-2-(5-methyl-thiophen-2-yl)-oxazol4-yl]-ethoxy}-benzyl)- amino]-acetic acid and its pharmaceutically acceptable salts; (Benzyl-{4-[2-(2,3-dihydro-benzo[1,4]thiazin-4-yl)-ethoxy]-benzyl}-amino)-acetic acid (Benzyl-(4-[2-(5-methyl-2-thiophen-3-yl-oxazol-4-yl)-ethoxy]-benzyl}-amino)-acetic acid and its pharmaceutically acceptable salts; ({4-[2-(2-Benzo[b]thiophen-2-yl-5-methyl-oxazol-4-yl)-ethoxy]-benzyl}-benzyl-amino)- acetic acid and its pharmaceutically acceptable salts; {Benzyl-[4-(3-methyl4-oxo-3,4-dihydro-quinazolin-2-ylmethoxy)-benzyl]-amino} -acetic acid and its pharmaceutically acceptable salts; {Benzyl-[4-(2-phenoxazin-10-yl-ethoxy)-benzyl]-amino}-acetic acid and its pharmaceutically acceptable salts; (Benzyl-{4-[2-(3,4-dihydro-2H-quinofin-1-yl)-ethoxy]-benzyl}-amino)-acetic acid and its pharmaceutically acceptable salts; [{4-[2-(5-Ethyl-pyridin-2-yl)-ethoxy]-benzyl}-(4-methoxy-phenoxycarbonyl)-amino]- acetic acid and its pharmaceutically acceptable salts; ((4-Methoxy-phenoxycarbonyl)-{4-[2-(methyl-pyridin-2-yl-amino)-ethoxy]-benzyl}- amino)-acetic acid and its pharmaceutically acceptable salts; [(4-Methoxy-phenoxycarbonyl)-(4- (2-[2-methyl-5-(5-methyl-thiophen-2-yl)-pyrrol- 1-yl]- ethoxy}-benzyl)-amino]-acetic acid and its pharmaceutically acceptable salts; ((4-Methoxy-phenoxycarbonyl)- (4-[2-(5-methyl-2-thiophen-2-yl-oxazol-4-yl)-ethoxy]- benzyl}-amino)-acetic acid and its pharmaceutically acceptable salts; [(4-Methoxy-phenoxycarbonyl)-(4-{2-[5-methyl-2-(5-methyl-thiophen-2-yl)-oxazol-4-yl]- ethoxy}-benzyl)-amino]-acetic acid and its pharmaceutically acceptable salts; {(4-Methoxy-phenoxycarbonyl)-[4-(3-methyl-4-oxo-3,4-dihydro-quinazolin-2- ylmethoxy)-benzyl]-amino}-acetic acid and its pharmaceutically acceptable salts; [[4-(2-Fluoro-benzyloxy)-benzyl]-(4-methoxy-phenoxycarbonyl)-amino]-acetic acid [(4-Methoxy-phenoxycarbonyl)-(4-{2-[2-methyl-5-(4-methylsulfanyl-phenyl)-pyrro-1-yl]- ethoxy}-benzyl)-amino]-acetic acid and its pharmaceutically acceptable salts; [{4-[2-(2-Furan-2-yl-5-methyl-oxazol-4-yl)-ethoxy]-benzyl}-(4-methoxy- phenoxycarbonyl)-amino]-acetic acid and its pharmaceutically acceptable salts; [{4-[2-(3-Ethyl-4-methyl-6-oxo-2-thioxo-3,6-dihydro-2H-pyrimidin- 1-yl)-ethoxy]- benzyl}-(4-methoxy-phenoxycarbonyl)-amino]-acetic acid and its pharmaceutically acceptable salts; ((4-Methoxy-phenoxycarbonyl)-(4-[2-(2,5,6-trimethyl-4-oxo-4H-thieno[2,3-d]pyrimidin- 3-yl)-ethoxy]-benzyl)-amino)-acetic acid and its pharmaceutically acceptable salts; [{4-[2-(3,4-Dihydro-2H-quinolin- 1-yl)-ethoxy]-benzyl) -(4-methoxy-phenoxycarbonyl)- amino]-acetic acid and its pharmaceutically acceptable salts; [(3-[2-(2-Furan-2-yl-5-methyl-oxazol-4-yl)-ethoxy]-benzyl}-(4-methoxy- phenoxycarboniyl)-amino]-acetic acid and its pharmaceutically acceptable salts; ((4-Methoxy-phenoxycarbonyl)-(4-[2-(methyl-pyrimidin-2-yl-amino)-ethoxy]-benzyl}- amino)-acetic acid and its pharmaceutically acceptable salts; [{4-[2-(2-Benzo[1,3]dioxol-5-yl-5-methyl-pyrrol-1-yl)-ethoxy]-benzyl}-(4-methoxy- phenoxycarbonyl)-amino]-acetic acid and its pharmaceutically acceptable salts; [[4-(2-Benzotriazol-1-yl-ethoxy)-benzyl]-(4-methoxy-phenoxycarbonyl)-amino]-acetic acid and its pharmaceutically acceptable salts; [(4-Methoxy-phenoxycarbonyl)-(4-{2-[5-methyl-2-(5-methyl-furan-2-yl)-oxazol-4-yl]- ethoxy}-benzyl)-amino]-acetic acid and its pharmaceutically acceptable salts; [{3-[2-(2-Benzo[1,3]dioxol-5-yl-5-methyl-pyrrol-1-yl)-ethoxy]-benzyl}-(4-methoxy- phenoxycarbonyl)-amino]-acetic acid and its pharmaceutically acceptable salts; [[4-(2-Benzoimidazol-1-yl-ethoxy)-benzyl]-(4-methoxy-phenoxycarbonyl)-amino]-acetic acid and its pharmaceutically acceptable salts; ((4-Methoxy-phenoxycarbonyl)-[4-(1-methyl-1H-benzoimidazol-2-ylmethoxy)-benzyl]- amino)-acetic acid and its pharmaceutically acceptable salts; [[4-(6-Methoxy-1-methyl-1H -benzoimidazol-2-ylmethoxy)-benzyl]-(4-methoxy- phenoxycarbonyl)-amino]-acetic acid and its pharmaceutically acceptable salts; [(4- {2-[2-(5-Bromo-thiophen-2-yl)-5-methyl-oxazol4-yl]-ethoxy}-benzyl)-(4-methoxy- phenoxycarbonyl)-amino]-acetic acid and its pharmaceutically acceptable salts; [(4-Methoxy-phenoxycarbonyl)-(3-{2-[2-methyl-5-(4-methylsulfanyl-phenyl)-pyrro1-1- yl]-ethoxy}-benzyl)-amino]-acetic acid and its pharmaceutically acceptable salts; [[4-(Benzothiazol-2-ylmethoxy)-benzyl]-(4-methoxy-phenoxycarbonyl)-amino]-acetic acid and its pharmaceutically acceptable salts; ((4-Methoxy-phenoxycarbonyl)- (4-[2-(5-methyl-3-phenyl-isoxazol-4-yl)-ethoxy]-benzyl}- amino)-acetic acid and its pharmaceutically acceptable salts; {(4-Methoxy-phenoxycarbonyl)-[4-(2-morpholin-4-yl-ethoxy)-benzyl]-amino}-acetic acid and its pharmaceutically acceptable salts; [(4-Methoxy-phenoxycarbonyl)-(4-{2-[mpethyl-(4-nitro-phenyl)-amino]-ethoxy}-benzyl)- amino]-acetic acid and its pharmaceutically acceptable salts; [(4-Methoxy-phenoxycarbonyl)-(3-{2-[2-methyl-5-(5-methyl-thiophen-2-yl)-pyrrol- 1-yl]- ethoxy}-benzyl)-amino]-acetic acid and its pharmaceutically acceptable salts; [[4-(Benzooxazol-2-ylmethoxy)-benzyl]-(4-methoxy-phenoxycarbonyl)-amino]-acetic acid and its pharmaceutically acceptable salts;

The present invention also provides novel processes for the preparation of compounds of formula (I) as well as intermediates involved in their synthesis. The compounds of the present invention can be prepared according to the general schemes provided below:

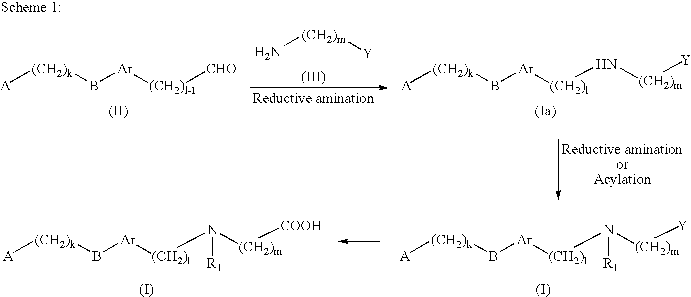

The compounds of general formula (I) wherein all the symbols are as defined earlier may be prepared by according to the process outlined in scheme 1 above, which comprises i) reacting the aldehyde of formula ([I) wherein all the symbols are as defined earlier with protected amino acids of formula (II) wherein Y denotes suitably protected carboxyl group such as an alkyl ester or amide and the like, in the presence of suitable reducing agents to obtain a compound of formula (la). Compound (Ia) represents compound of formula (I) where R₁=H. Suitable reducing agents like sodium borohydride, sodium triacetoxyborohydride, tetrabutyl ammonium borohydride and the like may be employed. The reaction may be carried out in solvents appropriate for the reagent used e.g. alcohols may be used with borohydrides and halogenated solvents such as 1,2-dichloroethane and the like or alcohols such as methanol, ethanol, propanol, isopropanol, butanol, t-butanol and the like or mixtures thereof may be used with sodium triacetoxyborohydride. Reaction temperatures may range from 0° C. to reflux temperature of the solvent(s) used. Reactions may be carried out in an atmosphere of inert gases like nitrogen, argon and the like but is not critical.

ii) compounds of general formula (Ia) wherein R₁=H & all other symbols are as defined earlier, may be converted to compounds of general formula (I) wherein all the symbols are as defined earlier by another reductive amination with an appropriate aldehyde (e.g. to get R₁=suitable alkyl derivative, corresponding aldehyde may be used; benzaldehyde may be used when R₁ is benzyl group) by a process similar to that described in (i) above or by acylation using appropriate acylating agents such as acyl halides (to get corresponding R₁=acyl), anhydrides (to get corresponding R₁=acyl), suitable carbamoyl chloride (to get NR₁=urea derivatives), suitable sulfonyl halides (to get R₁=sulfonates), haloformates (to get NR₁=carbamate) and the like. Reaction may be carried out in the presence of an inorganic base such as (aqueous) sodium(bi)carbonate, potassium(bi)carbonate, sodium or potassium hydroxide and the like or an organic base such as trialkyl amine, pyridine and the like. Solvents such as halogenated hydrocarbons (dichloromethane, dichloroethane, chloroform and the like), DMF, DMSO, ethers (diethyl ether, methyl tert butyl ether, tetrahydrofuran and the like) or mixtures thereof may be employed. Reaction temperatures may range from 0° C. to reflux temperature of the solvent(s) used. Reactions may be carried out in an atmosphere of inert gases like nitrogen, argon and the like but is not critical.

iii) the compound of general formula (I) (where R₃≢—OH), and all other symbols are as defined earlier may be optionally converted to compound of general formula (I) (R₃=—OH), by deprotecting the protected carboxyl group by using suitable deprotection methods e.g. acidic or basic hydrolysis may be employed when ‘Y’ is an ester. Aqueous alcohols and the like may be used as solvents. Reaction temperatures may range from 0° C. to reflux temperature of the solvent(s) used.
iv) optionally, if desired, the compounds of formula (I) are converted to their pharmaceutically acceptable salts by techniques known in the art.

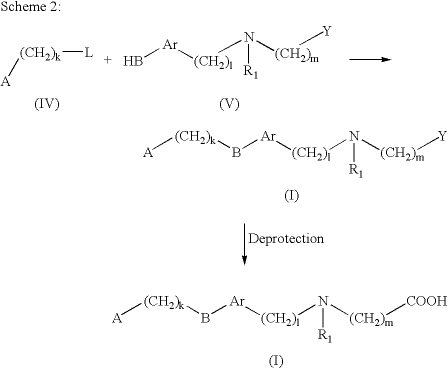

Alternatively, the compounds of general formula (I) wherein all the symbols are as defined earlier & R₃≢OH & NH₂ may be prepared by a sequence of reactions outlined in scheme 2 above which further may be converted to compound of general formula (I) by methods similar to those described in scheme 1 which comprises;

i) reacting compounds of general formula (IV) where L represents a suitable leaving group such as halogen, mesylate, tosylate, triflate & the like, with compounds of general formula (V) to obtain the compound of general formula (I). Suitable bases like metal hydrides e.g. Nag KH and the like, alkali metal carbonates e.g. potassium carbonate, sodium carbonate and the like, sodium hydroxide, potassium hydroxide, alkoxides such as NaOMe, NaOEt, potassium t-butoxide, sodium t-butoxide, sodium amyloxide and the like may be used. Reaction may be carried out in suitable solvents like DMF, DMSO, THF, toluene and the like or mixture thereof Acetone may also be used with alkali metal carbonates. Reaction temperature may range from 0° C. to the reflux temperature of the solvent(s) used. Inert atmosphere may be maintained using N₂, He, or argon gas. Reaction time may range from 1 to 72 hours.

ii) converting the compound of formula (I) to a further compound of formula (I) by the process as described in scheme I above.

iii) optionally, if desired, the compounds of formula I are converted to their pharmaceutically acceptable salts by techniques known in the art.

It will be appreciated that in any of the above-mentioned reactions any reactive group in the substrate molecule may be protected, according to conventional chemical practice. Suitable protecting groups in any of the above mentioned reactions are those used conventionally in the art. The methods of formation and removal in such protecting groups are those conventional methods appropriate to the molecule being protected. T. W. Greene and P. G. M. Wuts “Protective groups in Organic Synthesis”, John Wiley & Sons, Inc, 1999, 3^{rd} Ed., 201-245 along with references therein.

The pharmaceutically acceptable salts forming a part of this invention may be prepared by treating the compound of formula ( ) with 1-6 equivalents of a base such as sodium hydride, sodium methoxide, sodium ethoxide, sodium hydroxide, potassium tert-butoxide, calcium hydroxide, calcium acetate, calcium chloride, magnesium hydroxide, magnesium chloride, magnesium alkoxide and the like. Solvents such as water, acetone, ether, THF, methanol, ethanol, t-butanol, 2-butanone, dioxane, propanol, butanol, isopropanol, diisopropyl ether, tert-butyl ether or mixtures thereof may be used. Organic bases such as lysine, arginine, methyl benzylamine, ethanolamine, diethanolamine, tromethamine, choline, guanidine and their derivatives may be used. Acid addition salts, wherever applicable may be prepared by treatment with acids such as tartaric acid, mandelic acid, fumaric acid, malic acid, lactic acid, maleic acid, salicylic acid, citric acid, ascorbic acid, benzene sulfonic acid, p-toluene sulfonic acid, hydroxynaphthoic acid, methane sulfonic acid, acetic acid, benzoic acid, succinic acid, palmitic acid, hydrochloric acid, hydrobromic acid, sulfuric acid, nitric acid and the like in solvents such as water, alcohols, ethers, ethyl acetate, dioxane, THF, acetonitrile, DMF or a lower alkyl ketone such as acetone, or mixtures thereof.

Another aspect of the present invention comprises a pharmaceutical composition, containing at least one of the compounds of the general formula (I), their tautomeric forms, their pharmaceutically acceptable salts as an active ingredient, together with pharmaceutically employed carriers diluents and the like.

The novel compounds of the present invention can be formulated into suitable pharmaceutically acceptable compositions by combining with suitable excipients as are well known.

The novel compounds of the present invention can be formulated into suitable pharmaceutically acceptable compositions by combining with suitable excipients by techniques and processes and concentrations as are well known.

The compounds of formula (I) or pharmaceutical compositions containing them may be administered either by oral, topical or parenteral administration.

The pharmaceutical composition is provided by employing conventional techniques. Preferably’ the composition is in unit dosage form containing an effective amount of the active component, that is, the compounds of formula (I) according to this invention.

The quantity of active component, that is, the compounds of formula (I) according to this invention, in the pharmaceutical composition and unit dosage form thereof may be varied or adjusted widely depending upon the particular application method, the potency of the particular compound and the desired concentration. Generally, the quantity of active component will range between 0.5% to 90% by weight of the composition.

The compounds of general formula (I) or the compositions thereof are useful for the treatment and/or prophylaxis of disease caused by metabolic disorders such as hyperlipidemia, insulin resistance, leptin resistance, hyperglycernia, obesity, or inflammation

These compounds are useful for the treatment of hypercholesteremia, familial hypercholesteremia, hypertriglyceridemia, type 2 diabetes, dyslipidemia, disorders related to syndrome X such as hypertension, obesity, insulin resistance, coronary heart disease, atherosclerosis, xanthoma, stroke, peripheral vascular diseases and related disorders and diabetic complications.

The compounds of the invention may be administered to a mammal, especially, a human in need of such treatment, prevention, elimination, alleviation or amelioration of diseases mentioned above.

In another aspect of the present invention, method of treatment and/or prevention of the diseases mentioned above by treatment with compounds of the present invention are provided.

In a further aspect of the present invention, use of one or more compounds of the general formula (I) or pharmaceutically acceptable salts, for the preparation of a medicament thereof for the treatment and/or prevention of diseases mentioned in this document is provided.

The invention is explained in detail by the examples given below, which are provided by way of illustration only and therefore should not be construed to limit the scope of the invention.

The invention is explained in detail by the examples given below, which are provided by way of illustration only and therefore should not be construed to limit the scope of the invention.

1H NMR spectral data given in the tables (vide infra) are recorded using a 300 MHz spectrometer (Bruker AVANCE-300) and reported in δ scale. Until and otherwise mentioned the solvent used for NMR is CDCl₃ using Tetramethyl silane as the internal standard.

### Preparation 1

### Ethyl-{4-[2-(2,3-dihydro-benzo[1,4]oxazin-4-yl)-ethoxy]benzyl amino) acetate.

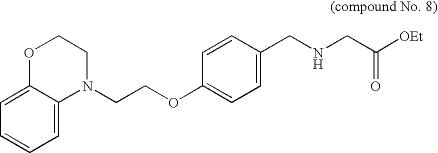

To a solution of 4-[2-(2,3-dihydro-benzo [1,4]oxazin-4-yl)ethoxybenzaldehyde (2.3 g) and glycine ethyl ester hydrochloride (1.2 g) in methanol (40 mL) was added triethyl amine (1.3 mL) at 30° C. and the reaction mixture was cooled in an ice bath. To this was added sodium borohydride (370 mg) and stirred for one hour at 30° C. The solvent was evaporated under reduced pressure and the residue was extracted with ethyl acetate (3×50 mL). The combined organic extract was washed with water (100 mL), brine solution (75 mL), dried over sodium sulphate and evaporated under reduced pressure. The crude product was flash chromatographed over silica gel using 25-40% ethyl acetate in petroleum ether as an eluent to yield 1.35 g of pure product.

### Preparation 2

### Ethyl-[4-(2-phenothiazin- 10-yl-ethoxy)-benzylamino]-acetate.

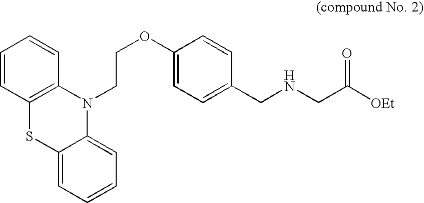

To a solution of 4-(2-phenothiazin-10-yl-ethoxy)-benzaldehyde (1.4 g) and glycine ethyf ester hydrochloride (0.59 g) in ethanol (30 mL) was added triethyl amine (0.64 mL) and the reaction mixture was stirred at 30 ° C. for 17 hours. The reaction mixture was cooled in an ice-bath and sodium borohydride (176 mg) was added to it in portions. Stirring was continued for 15 hours at 30° C. The solvent was evaporated under reduced pressure and the residue was dissolved in chloroform (150 mL), washed with water (100 mL), dried over calcium chloride and evaporated under reduced pressure to yield the product (2.0 g). The following compounds in table 1 were prepared following the procedure similar to that described for preparation 1-2.

TABLE 1

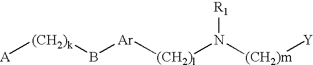

| | | | | | | | | | Mol. | % |
|---|---|---|---|---|---|---|---|---|---|---|
| S. No | A | Ar | R₁ | Y | B | k | 1 | m | Wt. | Yield |
| 1. | 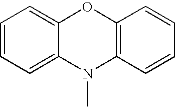 | 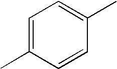 | H | COOEt | O | 2 | 1 | 1 | 418 | 71 |
| | ¹H: 1.27 (3H, t, J=7.14 Hz), 3.38 (2H, s), 3.73 (2H, s), 3.96 (2H, t, J=6.6 Hz), 4.2 (4H, m), | | | | | | | | | |
| | 6.61-6.69 (6H, m), 6.76-6.87 (4H, m), 7.22 (2H, d, J=9.14 Hz). | | | | | | | | | |
| 2. | 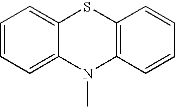 | 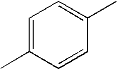 | H | COOEt | O | 2 | 1 | 1 | 434 | 73 |
| | ¹H: 1.26 (3H, t, J=7.13 Hz), 3.37 (2H, s), 3.72 (2H, s), 3.96 (2H, q, J=7.13 Hz), 4.30 (4H, s), | | | | | | | | | |
| | 6.84 (2H, d, J=8.6 Hz), 6.9 (4H, d, J=7.8 Hz), 7.13 (4H, d, J=7.58 Hz), 7.25 (2H, d, J=8.58 Hz). | | | | | | | | | |
| 3. | 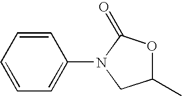 | 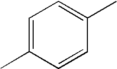 | H | COOMe | O | 1 | 1 | 1 | 370 | 60 |
| | ¹H: 3.4 (2H, t, J=5.6 Hz), 3.72 (3H, s), 3.74 (2H, s), 4.07 (1H, dd, J=8.9 & 6.0 Hz), 4.20-4.25 | | | | | | | | | |
| | (3H, m), 4.98 (1H, m), 6.85 (2H, d, J=8.6 Hz), 7.15 (1H, t, J=7.3 Hz), 7.26 (2H, m), 7.36-7.41 | | | | | | | | | |
| | (2H, m), 7.56 (2H, d, J=7.83 Hz). | | | | | | | | | |
| 4. | 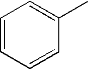 | 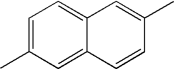 | H | COOEt | O | 1 | 1 | 1 | 349 | 25 |
| | ¹H: 1.25 (3H, t, J=7.14 Hz), 3.44 (2H, s), 3.9 (2H, s), 4.2 (2H, q, J=7.14 Hz), 5.18 (2H, s), 7.2 | | | | | | | | | |
| | (2H, m), 7.34-7.44 (4H, m), 7.48 (2H, d, J=7.1 Hz), 7.69-7.74 (3H, m). | | | | | | | | | |
| 5. | 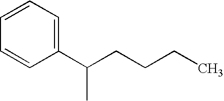 | 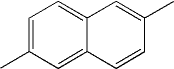 | H | COOEt | O | 0 | 1 | 1 | 405 | 52 |
| | ¹H: 0.9 (3H, t, J=7.1 Hz), 1.25 (3H, t, J=7.1 Hz), 1.4 (4H, complex), 1.8 (1H, m), 2.0 (1H, m), | | | | | | | | | |
| | 3.39 (2H, s), 3.88 (2H, s), 4.18 (2H, q, J=7.14 Hz), 5.2 (1H, m), 6.98 (1H, d, J=2.22 Hz), 7.2 | | | | | | | | | |
| | (2H, m), 7.3 (3H, m), 7.4 (2H, d, J=7.08 Hz), 7.5 (1H, J=8.43 Hz), 7.64 (2H, t, J=7.58 Hz). | | | | | | | | | |
| 6. | 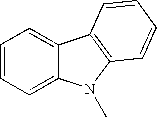 | 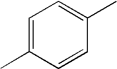 | H | COOEt | O | 2 | 1 | 1 | 402 | 59 |
| | ¹H: 1.2 (3H, t, J=7.1 Hz), 3.3 (2H, s), 3.7 (2H, s), 4.2 (2H, q, J₁=J₂=7.1 Hz), 4.3 (2H, t, J=6.0 | | | | | | | | | |
| | Hz), 4.7 (2H, t, J=6.1 Hz), 6.7 (2H, d, J=8.6 Hz), 7.1 (2H, d, J=8.6 Hz), 7.2 (2H, m), 7.4-7.5 | | | | | | | | | |
| | (4H, m), 8.1 (2H, d, J=7.8 Hz). | | | | | | | | | |
| 7. | 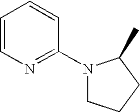 | 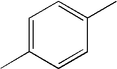 | H | COOEt | O | 1 | 1 | 1 | 369 | 64 |
| | ¹H: 1.2 (3H, t, J=7.1 Hz), 2.0 (2H, m), 2.2 (2H, m), 3.3 (1H, m), 3.4 (2H, s), 3.5 (1H, t, J=8.7 | | | | | | | | | |
| | Hz), 3.7 (2H, s), 3.8 (1H, t, J=8.7 Hz), 4.1 (2H, q, J₁=J₂=7.1 Hz), 4.2 (1H, dd, J=9.1 & 3.2 Hz), | | | | | | | | | |
| | 4.4 (1H, m), 6.4 (1H, d, J=8.5 Hz), 6.5 (1H, m), 6.9 (2H, d, J=8.6 Hz), 7.2 (2H, d, J=8.55 Hz), | | | | | | | | | |
| | 7.4 (1H, m), 8.1 (1H, dd, J=4.9 & 1.1 Hz). | | | | | | | | | |
| 8. | 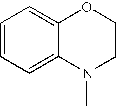 | 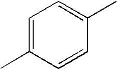 | H | COOEt | O | 2 | 1 | 1 | 370 | 30 |

¹H: 1.27 (3H, t, J=5.95 Hz), 3.38 (2H, s), 3.51 (2H, t, J=4.41 Hz), 3.69 (2H, t, J=5.7 Hz), 3.72
(2H, s), 4.14-4.24 (6H, complex), 6.61 (1H, m), 6.68 (1H, dd, J=6.84 & 8.14 Hz), 6.77-6.85
(4H, complex), 7.24 (2H, t, J=7.05 Hz).

### Preparation 3

### Ethyl-{benzyl-[3-(2-carbazol-9-yl-ethoxy)-benzyl]-amino}-acetate.

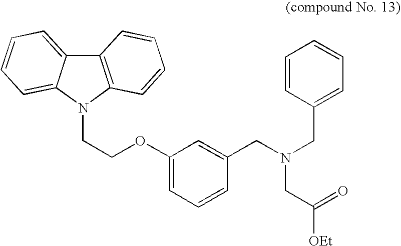

To a solution of ethyl-[3-(2-carbazol-9-yl-ethoxy)-benzyl]-amino)-acetate (compound No.6) (1.5 g) and benzaldehyde (0.4 mL) in dichloromethane (20 mL) was added sodium triacetoxy borohydride (1.0 g) and the reaction mixture was stirred at 30° C. for 36 hours. The reaction mixture was diluted with dichloromethane (75 mL) and washed with water (2X100 rnL). The organic extract was dried over calcium chloride and evaporated under reduced pressure. The crude product was chromatographed (flash) over silica gel using 10% ethyl acetate in petroleum ether as an eluent to yield 0.8 g of pure product.

### Preparation 4

### Ethyl-{benzyl-[3-(2-indol-1-yl-ethoxy)-benzyl]-amino}-acetate.

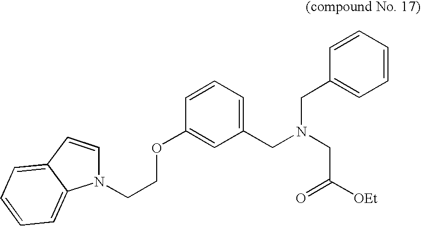

A mixture of 2-(indole-1-yl)-ethyl methanesulfonate (1.0 g), ethyl-{benzyl-[3- hydroxy benzyl]-amino}-acetate (1.25 g), potassium carbonate (1.5 g) and tetrabutylammonium bromide (10 mg) in dimethyl formamide was stirred at 60° C. for 17 hours. The reaction mixture was cooled to 30° C. and poured in to ice cold water (150 mL) and extracted with diethyl ether (3×50 mL). The combined organic extract was washed with water (100 mL), brine solution (100 mL), dried over sodium sulphate and evaporated under reduced pressure. The crude product was (flash) chromatographed over silica gel using 10% ethyl acetate in petroleum ether as an eluent to yield 480 mg of pure product.

### Preparation 5

### Ethyl-[{4-[2-(2,3-dihydro-benzo[1,4]oxazin-4-yl)-ethoxy]-benzyl}(4methoxyphenoxycarbonyl)-amino]-acetate. (compound No.32)

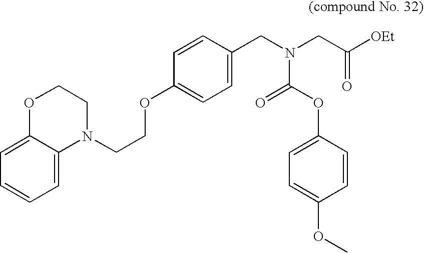

To an ice cold solution of ethyl-{4-[2-(2,3-dihydro-benzo[1,4]oxazin-4-yl)-ethoxy]benzyl amino} acetate (compound No. 8) (0.9 g), and 4-methoxyphenyl chloroformate (0.5 g) in dichloromethane (5 mL) was added pyridine (0.25 mL) and the reaction mixture was stirred at ca. 0° C. for 30 minutes. The reaction mixture was diluted with dichloromethane (50 mL), washed with water (25 mL), 1.0 N HCl (25 mL) followed by water (25 mL), dried over calcium chloride and evaporated under reduced pressure. The crude product was chromatographed over silica gel using 10-20% ethyl acetate in petroleum ether as an eluent to yield 0.5 g pure product.

### Preparation 6

### Ethyl-[{4-[2-(2,3-dihydro-benzo[1,4]thiazin-4-yl)-ethoxy]-benzyl}-(4-methoxy-phenoxycarbonyl)-amino]-acetate.(compound No.28)

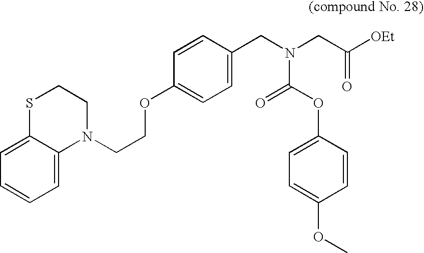

A mixture of 2-((2,3-dihydro-benzo[1,4]thiazin-4-yl)-ethyl}methane sulfonate (1 g), ethyl-[(4-hydroxy benzyl}-(4-methoxy-phenoxycarbonyl)-amnino]-acetate (1.4 g) and potassium carbonate (1.0 g) in 10 mL of dimethyl formamide was stirred at 70° C. after adding 30 mg of tetrabutyl ammonium bromide. The reaction mixture was cooled to 30° C. and water (150 mL) was added. This was extracted with ethyl acetate (3×50 mL). The combined organic extract was washed with water (2×100 mL), brine solution (100 mL), dried over sodium sulphate and evaporated under reduced pressure. The crude product was chromatographed over silica gel using 10-20% ethyl acetate in petroleum ether as an eluent to yield 1.7 g of pure product.

### Preparation 7

### Ethyl-(ethoxycarbonylmethyl-{4-[2-(5-methyl-2-phenyl-oxazol4-yl)-ethoxy]- benzyl}-amino)-acetate.

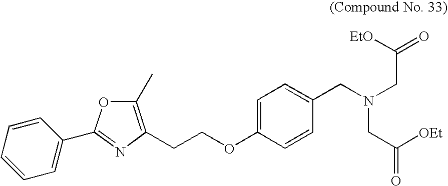

To a stirred and ice cold suspension of 60% sodium hydride (183 mg) in dry THF (5 mL) was added a solution of ethyl-{4-[2-(5-methyl-2-phenyl-oxazol-4-yl)-ethoxy]- benzylamino}-acetate (1.0 g) in dry THF (5 mL) and the reaction mixture was stirred at 30° C. for 3 hours. The reaction mixture was again cooled in an ice-bath and to it was added ethyl bromoacetate (0.3 mL) and the reaction mixture was stirred at 30° C. for 18 hours. The reaction mixture was poured into ice cold water (100 mL) and extracted with ethyl acetate (3×50 mL). The combined organic extract was washed with water (50 mL), brine solution (50 mL), dried over sodium sulphate and evaporated under reduced pressure. The crude product was flash chromatographed over silica gel using 5% ethyl acetate in petroleum ether as an eluent to obtain 1.1 g of pure product.

### Preparation 8

### Ethyl-[{4-[2-(6,7-dihydro-4H-thieno[3,2-c]pyridin-5-yl)-ethoxy]-benzyl}-(4-methoxy-phenoxycarbonyl)-amino]-acetate (compound.No.27).

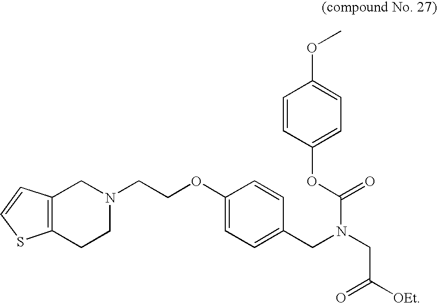

To an ice cold and stirred mixture of Ph₃P (0.95 g) and diisopropyl azodicarboxylate (0.71 mL) in dry THF (5 mL) was added a solution of 2-(6,7-dihydro-4H-thieno[3,2-c]pyridin-5-yl)-ethanol (0.5 g) in THF, followed by a solution of ethyl-[(4-hydroxy-benzyl)-(4-methoxy-phenoxycarbonyl)-amino]-acetate (1.0 g) in THF and the reaction mixture was stirred at 30° C. for 1.5 hours. The reaction mixture was poured in to ice cold water (50 mL) and extracted with ethyl acetate (3×50 mL). The combined organic extract was washed with water (50 mL), brine solution (50 mL), dried over sodium sulphate and evaporated under reduced pressure to yield 1.0 g crude product which was hydrolysed as such without purification.

In like manner compounds in table 2 were prepared by a procedure similar to that described in preparation 3-8.

TABLE 2

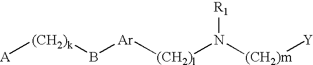

| S. | | | | | | | | | Mol. | % |
|---|---|---|---|---|---|---|---|---|---|---|
| No | A | Ar | R₁ | Y | B | k | 1 | m | Wt. | Yield |
| 9. | 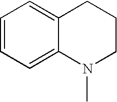 | 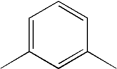 | 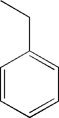 | COOEt | O | 2 | 1 | 1 | 458 | 31 |
| | ¹H: 1.25 (3H, t, J=6.05 Hz), 1.95 (2H, m), 2.75 (2H, t, J=6.27 Hz), 3.29 (2H, s), 3.43 (2H, t, | | | | | | | | | |
| | J=5.61 Hz), 3.69 (2H, t, J=6.54 Hz), 3.77 (2H, s), 3.79 (2H, s), 4.14 (4H, m), 6.61 (2H, m), | | | | | | | | | |
| | 6.76 (1H, m), 6.94 (3H, m), 7.0 (1H, t, J=7.5 Hz), 7.2-7.4 (6H, complex). | | | | | | | | | |
| 10. | 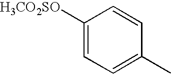 | 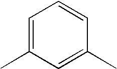 | 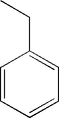 | COOEt | O | 2 | 1 | 1 | 497 | 95 |
| | ¹H: 1.2 (3H, t, J=7.6 Hz), 3.0 (2H, t, J=6.7 Hz), 3.1 (3H, s), 3.2 (2H, s), 3.79 (2H, s), 3.83 (2H, | | | | | | | | | |
| | s), 4.2 (4H, m), 6.9 (3H, m), 7.2 (4H, m), 7.6 (6H, m). | | | | | | | | | |
| 11. | 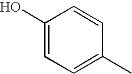 | 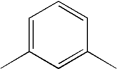 | 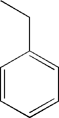 | COOEt | O | 2 | 1 | 1 | 419 | 49 |
| | ¹H: 1.25 (3H, t, J=3.55 Hz), 3.0 (2H, t, J=7.01 Hz), 3.28 (2H, s), 3.77 (2H, s), 3.79 (2H, s), | | | | | | | | | |
| | 4.13 (4H, m), 6.9 (3H, m), 7.2 (4H, m), 7.6 (6H, m). | | | | | | | | | |
| 12. | 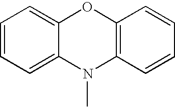 | 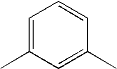 | 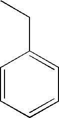 | COOEt | O | 2 | 1 | 1 | 508 | 46 |
| | ¹H: 1.24 (3H, t, J=6.9 Hz), 3.2 (2H, s), 3.77 (2H, s), 3.79 (2H, s), 3.97 (2H, t, J=6.6 Hz), 4.17 | | | | | | | | | |
| | (4H, m), 6.6 (6H, m), 6.78 (3H, m), 6.98 (2H, m), 7.2-7.3 (6H, complex). | | | | | | | | | |
| 13. | 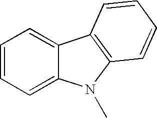 | 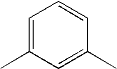 | 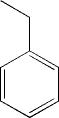 | COOEt | O | 2 | 1 | 1 | 492 | 43 |
| | ¹H: 1.23 (3H, t, J=7.14 Hz), 3.25 (2H, s), 3.73 (2H, s), 3.76 (2H, s), 4.11 (2H, q, J=7.2 Hz), | | | | | | | | | |
| | 4.36 (2H, t, J=6.5 Hz), 4.72 (2H, t, J=6.01 Hz), 6.7 (1H, d, J=8.1 & 2.16 Hz), 6.8 (1H, s), 6.92 | | | | | | | | | |
| | (2H, m), 7.17-7.5 (5H, complex), 7.48 (6H, m), 8.1 (2H, d, J=7.74 Hz). | | | | | | | | | |
| 14. | 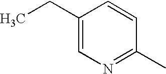 | 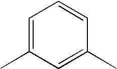 | 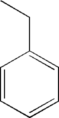 | COOEt | O | 2 | 1 | 1 | 432 | 88 |
| | ¹H: 1.24 (6H, t), 2.62 (2H, q, J=7.6 Hz), 3.25 (2H, t, J=6.7 Hz), 3.27 (2H, s), 3.76 (2H, s), 3.79 | | | | | | | | | |
| | (2H, s), 4.1 (2H, q, J=7.12 Hz), 4.34 (2H, t, J=6.7 Hz), 6.77 (1H, dd, J=9.4 & 2.1 Hz), 6.94 | | | | | | | | | |
| | (2H, d, J=7.59 Hz), 7.2-7.4 (7H, complex), 7.46 (1H, dd, J=2.16 & 7.89 Hz), 8.4 (1H, s). | | | | | | | | | |
| 15. | 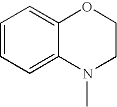 | 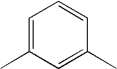 | 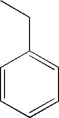 | COOEt | O | 2 | 1 | 1 | 460 | 79 |
| | ¹H: 1.2 (3H, t, J=7.14 Hz), 3.27 (2H, s), 3.5 (2H, t, J=4.4 Hz), 3.7 (2H, t, J=5.7 Hz), 3.77 (2H, | | | | | | | | | |
| | s), 3.79 (2H, s), 4.1-4.18 (4H, m), 4.2 (2H, t, J=4.4 Hz), 6.6 (1H, m), 6.7 (1H, m), 6.8 (3H, m), | | | | | | | | | |
| | 6.9 (2H, d, J=7.89 Hz), 7.2-7.38 (6H, m). | | | | | | | | | |
| 16. | 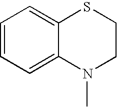 | 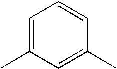 | 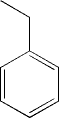 | COOEt | O | 2 | 1 | 1 | 467 | 88 |
| | ¹H: 1.2 (3H, t, J=7.13 Hz), 3.0 (2H, m), 3.28 (2H, s), 3.7-3.8 (8H, m), 4.1 (4H, m), 6.6 (1H, t, | | | | | | | | | |
| | J=7.4 Hz), 6.7 (2H, m), 6.9 (2H, d, J=7.3 Hz), 7.0 (2H, dd, J=7.6 & 1.4 Hz), 7.18-7.38 (6H, | | | | | | | | | |
| | m). | | | | | | | | | |
| 17. | 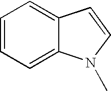 | 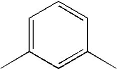 | 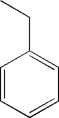 | COOEt | O | 2 | 1 | 1 | 442 | 54 |
| | ¹H: 1.2 (3H, t, J=7.1 Hz), 3.26 (2H, s), 3.74 (2H, s), 3.77 (2H, s), 4.1 (2H, q, J=7.1 Hz), 4.3 | | | | | | | | | |
| | (2H, t, J=5.64 Hz), 4.5 (2H, t, J=5.64 Hz), 6.5 (1H, d, J=3 Hz), 6.7 (1H, dd, J=2.1 & 8.1 Hz), | | | | | | | | | |
| | 7.1 (2H, m,), 7.0-7.23 (5H, m), 7.29-7.42 (5H, m), 7.6 (1H, d, J=8.16 Hz). | | | | | | | | | |
| 18. | 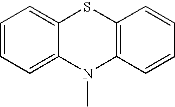 | 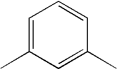 | 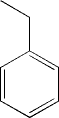 | COOEt | O | 3 | 1 | 1 | 538 | 100 |
| | ¹H: 1.2 (3H, t, J=7.1 Hz), 2.27 (2H, m), 3.27 (2H, s), 3.75 (2H, s), 3.79 (2H, s), 4.07-4.17 (6H, | | | | | | | | | |
| | m), 675 (1H, m), 6.88-6.95 (6H, m), 7.12-7.17 (5H, m), 7.22 (1H, d, J=7.78 Hz), 7.30 (2H, t, | | | | | | | | | |
| | J=7.1 Hz), 7.38 (2H, d, J=7.2 Hz). | | | | | | | | | |
| 19. | 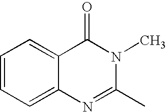 | 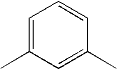 | 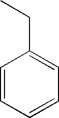 | COOEt | O | 1 | 1 | 1 | 471 | 56 |
| | ¹H: 1.2 (3H, t, J=7.1 Hz), 3.2 (2H, s), 3.74 (3H, s), 3.79 (4H, s), 4.1 (2H, q, J=7.0 Hz), 5.18 | | | | | | | | | |
| | (2H, s), 6.9 (1H, dd), 7.0 (1H, d, J=7.5 Hz), 7.1 (1H, s), 7.2-7.3 (6H, complex), 7.5 (1H, t), | | | | | | | | | |
| | 7.7 (2H, m), 8.2 (1H, d, J=7.6 Hz). | | | | | | | | | |
| 20. | 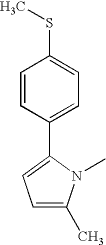 | 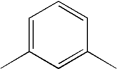 | 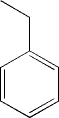 | COOEt | O | 2 | 1 | 1 | 528 | 50 |
| 21. | 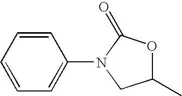 | 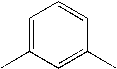 | 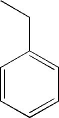 | COOEt | O | 1 | 1 | 1 | 474 | 85 |
| | ¹H: 1.2 (3H, t, J=7.1 Hz), 3.3 (2H, s), 3.77 (2H, s), 3.79 (2H, s), 4.0-4.2 (6H, m), 4.9 (1H, m), | | | | | | | | | |
| | 6.8 (1H, m), 7.0 (2H, m), 7.1-7.4 (9H, complex), 7.5 (2H, d, J=8.5 Hz). | | | | | | | | | |
| 22. | 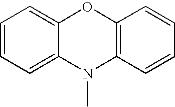 | 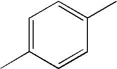 | 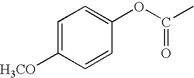 | COOEt | O | 2 | 1 | 1 | 568 | 100 |
| | ¹H: 1.25 (3H, t, J=7.0 Hz), 3.72 (3H, s), 3.98 (4H, m), 4.19 (2H, m), 4.56 (1H, s), 4.66 (1H, s), | | | | | | | | | |
| | 4.74 (2H, m), 6.61-6.67 (6H, m), 6.77-6.92 (6H, m), 7.04 (2H, t, J=8.8 Hz), 7.16-7.22 (2H, | | | | | | | | | |
| | m). | | | | | | | | | |
| 23. | 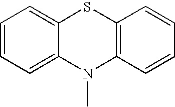 | 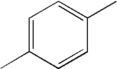 | 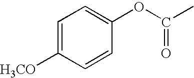 | COOEt | O | 2 | 1 | 1 | 584 | 89 |
| | ¹H: 1.25 (3H, t, J=7.1 Hz), 3.80 (3H, s), 4.0 (2H, m), 4.19 (2H, q, J=7.1 Hz), 4.31 (4H, s), 4.55 | | | | | | | | | |
| | (1H, s), 4.66 (1H, s), 6.85-6.96 (8H, m), 7.04 (3H, m), 7.16 (4H, d, J=7.46 Hz), 7.2 (1H, m). | | | | | | | | | |
| 24. | 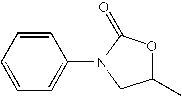 | 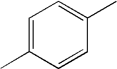 | 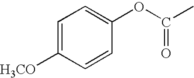 | COOMe | O | 1 | 1 | 1 | 520 | 94 |
| | ¹H: 3.79 (3H, s), 3.80 (3H, s), 4.0 (2H, d, J=3.42 Hz), 4.07 (1H, m), 4.17-4.24 (3H, m), 4.57 | | | | | | | | | |
| | (1H, s), 4.67 (1H, s), 5.0 (1H, m), 6.85-6.92 (5H, m), 7.04 (2H, t, J=8.8 Hz), 7.13-7.19 (2H, | | | | | | | | | |
| | m), 7.40 (2H, t, J=8.0 Hz), 7.56 (2H, d, J=8.0 Hz). | | | | | | | | | |
| 25. | 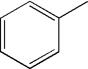 | 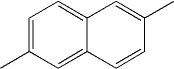 | 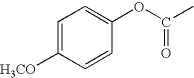 | COOEt | O | 1 | 1 | 1 | 499 | 69 |
| | ¹H: 1.25 (3H, t, J=7.1 Hz), 3.79 (3H, s), 4.03 (2H, s), 4.15 (2H, q, J=7.1 Hz), 4.76 (1H, s), 4.87 | | | | | | | | | |
| | (1H, s), 5.18 (2H, s), 6.9 (2H, d, J=8.76 Hz), 7.07 (2H, m), 7.22 (2H, m), 7.32-7.43 (4H, m), | | | | | | | | | |
| | 7.5 (2H, d, J=7.13 Hz), 7.66 (1H, s), 7.72 (2H, t, J=7.36 Hz). | | | | | | | | | |
| 26. | 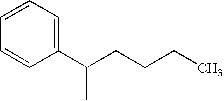 | 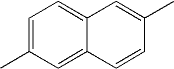 | 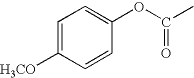 | COOMe | O | 0 | 1 | 1 | 541 | 60 |
| | ¹H: 0.9 (3H, t, J=7.1 Hz), 1.36 (4H, m), 1.88 (1H, m), 2.05 (1H, m), 3.7 (3H, s), 3.79 (3H, s), | | | | | | | | | |
| | 4.0 (2H, s), 4.71 (1H, s), 4.8 (1H, s), 5.2 (H, t, J=6.3 Hz), 6.8 (2H, d, J=8.3 Hz), 6.99-7.08 (3H, | | | | | | | | | |
| | m), 7.18-7.23 (2H, m), 7.32 (3H, t, J=7.36 Hz), 7.4 (2H, d, J=8.05 Hz), 7.57 (2H, m), 7.68 | | | | | | | | | |
| | (1H, d, J=8.9 Hz). | | | | | | | | | |
| 27. | 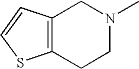 | 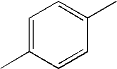 | 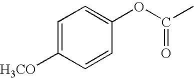 | COOEt | O | 2 | 1 | 1 | 524 | 87 |
| 28. | 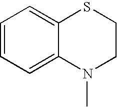 | 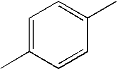 | 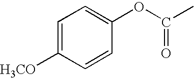 | COOEt | O | 2 | 1 | 1 | 536 | 90 |
| | ¹H: 1.25 (3H, t, J=7.1 Hz), 3.0 (2H, t, J=5.0 Hz), 3.74 (4H, m), 3.79 (3H, s), 3.98 (2H, m), | | | | | | | | | |
| | 4.17 (4H, m), 4.56 (1H, s), 4.67 (1H, s), 6.62 (1H, t, J=7.33 Hz), 6.72 (1H, d, J=8.16 Hz), 6.87 | | | | | | | | | |
| | (4H, m), 6.96-7.07 (4H, m) 7.21 (2H, m). | | | | | | | | | |
| 29. | 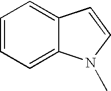 | 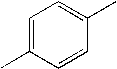 | 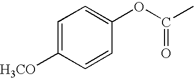 | COOEt | O | 2 | 1 | 1 | 502 | 73 |
| | ¹H: 1.2 (3H, t, J=7.1 Hz), 3.78 (3H, s), 3.96 (2H, s), 4.1 (2H, m), 4.3 (2H, m), 4.5 (3H, m), 4.6 | | | | | | | | | |
| | (1H, s), 6.5 (1H, d, J=3.09 Hz), 6.8-6.9 (4H, m), 7.0 (2H, m), 7.1 (1H, t, J=7.44 Hz), 7.2 (4H, | | | | | | | | | |
| | m), 7.4 (1H, d, J=8.32 Hz), 7.6 (1H, d, J=7.8 Hz). | | | | | | | | | |
| 30. | 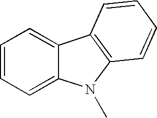 | 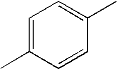 | 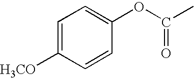 | COOEt | O | 2 | 1 | 1 | 552 | 97 |
| | ¹H: 1.2 (3H, t, J=7.1 Hz), 3.75 (2H, m), 3.79 (3H, s), 4.1-4.2 (2H, m), 4.3 (2H, m), 4.5 (1H, s), | | | | | | | | | |
| | 4.6 (1H, s), 4.7 (2H, m), 6.7 (2H, m), 6.8-6.9 (3H, m), 7.0 (2H, m), 7.1 (2H, m), 7.2 (1H, m), | | | | | | | | | |
| | 7.5 (4H, m), 8.1 (2H, d, J=7.75 Hz). | | | | | | | | | |
| 31. | 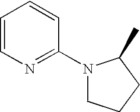 | 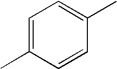 | 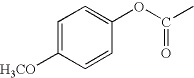 | COOEt | O | 1 | 1 | 1 | 519 | 81 |
| | ¹H: 1.20-1.28 (3H, t, J=7.1 Hz), 2.0 (2H, m), 2.2 (2H, m), 3.3 (1H, m), 3.5 (1H, m), 3.8 (3H, | | | | | | | | | |
| | s), 3.8 (1H, m), 3.98 (2H, d, J=1.9 Hz), 4.17 (2H, q, J=7.14 Hz), 4.26 (1H, m), 4.5 (1H, m), | | | | | | | | | |
| | 4.55 (1H, s), 4.66 (1H, s), 6.4 (1H, d, J=8.5 Hz), 6.5 (1H, m), 6.8 (2H, m), 6.9-7.4 (4H, m), 7.2 | | | | | | | | | |
| | (2H, dd, J=8.7 & 2.5 Hz), 7.4 (1H, m), 8.1 (1H, m). | | | | | | | | | |
| 32. | 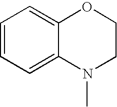 | 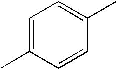 | 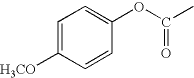 | COOEt | O | 2 | 1 | 1 | 520 | 80 |
| | ¹H: 1.25 (3H, t, J=7.1 Hz), 3.51 (2H, t, J=4.4 Hz), 3.70 (2H, m), 3.79 (3H, s), 4.0 (2H, m), | | | | | | | | | |
| | 4.15-4.2 (6H, m), 4.56 (1H, s), 4.66 (1H, s), 6.62 (1H, m), 6.7 (1H, d, J=8.01 Hz), 6.79 (1H, | | | | | | | | | |
| | m), 6.8 (5H, m), 7.0 (2H, t, J=9.54 Hz), 7.2 (2H, m). | | | | | | | | | |
| 33. | 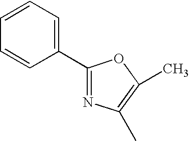 | 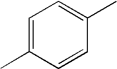 | 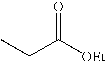 | COOEt | O | 2 | 1 | 1 | 480 | 90 |
| | ¹H: 1.25 (6H, t, J=7.1 Hz), 2.37 (3H, s), 2.97 (2H, t, J=6.6 Hz), 3.51 (4H, s), 3.82 (2H, s), 4.1 | | | | | | | | | |
| | (4H, q, J=7.1 Hz), 4.23 (2H, t, J=6.6 Hz), 6.8 (2H, d, J=8.5 Hz), 7.2 (2H, d, J=8.5 Hz), 7.35 | | | | | | | | | |
| | (3H, m), 7.98 (2H,d, J=7.8 Hz). | | | | | | | | | |
| 34. | 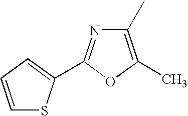 | 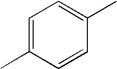 | 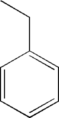 | COOEt | O | 2 | 1 | 1 | 490 | 60 |
| | ¹H: 1.25 (3H, t, J=7.12 Hz), 2.34 (3H, s), 2.94 (2H, t, J=6.58 Hz), 3.24 (2H, s), 3.72 (2H, s), | | | | | | | | | |
| | 3.77 (2H, s), 4.10-4.17 (2H, q, J=7.16 Hz), 4.2 (2H, t, J=6.61 Hz), 6.82-6.85 (2H, d, J=8.52 | | | | | | | | | |
| | Hz), 7.05-7.08 (1H, m), 7.20-7.37 (8H, m), 7.56-7.58 (1H, m). | | | | | | | | | |
| 35. | 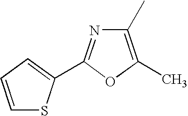 | 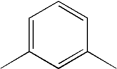 | 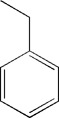 | COOEt | O | 2 | 1 | 1 | 490 | 60 |
| 36. | 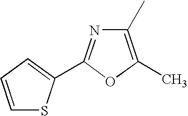 | 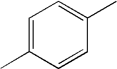 | 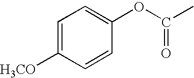 | COOEt | O | 2 | 1 | 1 | 550 | 44 |
| | ¹H: 1.25 (3H, t, J=7.11 Hz), 2.36 (3H, s), 2.95 (2H, t, J=6.48 Hz) 3.79 (3H, s), 3.97 (2H, s), | | | | | | | | | |
| | 4.14-4.24 (4H, m), 4.55 (1H, s), 4.66 (1H, s), 6.85-6.90 (4H, m), 7.01-7.09 (3H, m), 7.20-7.22 | | | | | | | | | |
| | (2H, m), 7.35 (1H, m), 7.57 (1H, m). | | | | | | | | | |
| 37. | 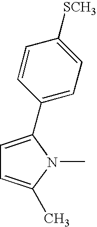 | 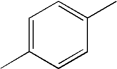 | 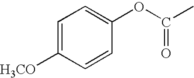 | COOEt | O | 2 | 1 | 1 | 588 | 56 |
| | ¹H: 1.27 (3H, t, J=7.25 Hz), 2.37 (3H, s), 2.51 (3H, s), 3.79 (3H, s), 3.92-3.97 (4H, m), 4.17-4.19 | | | | | | | | | |
| | (2H, m), 4.28 (2H, t, J=6.4 Hz), 4.53 (1H, s), 4.64 (1H, s), 5.97 (1H, d, J=3.21 Hz), 6.10 | | | | | | | | | |
| | (1H, d, J=3.36 Hz), 6.63-6.68 (2H, m), 6.88 (2H, d, J=8.85 Hz), 7.01-7.06 (2H, m), 7.15-7.18 | | | | | | | | | |
| | (2H, m), 7.289-7.34 (4H, m). | | | | | | | | | |
| 38. | 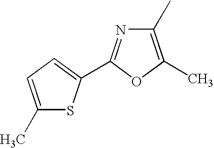 | 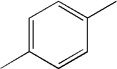 | 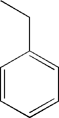 | COOEt | O | 2 | 1 | 1 | 504 | 30 |
| 39. | 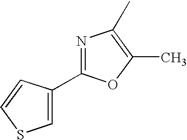 | 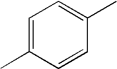 | 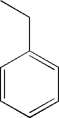 | COOEt | O | 2 | 1 | 1 | 490 | 30 |
| 40. | 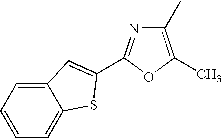 | 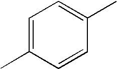 | 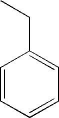 | COOEt | O | 2 | 1 | 1 | 540 | 25 |
| 41. | 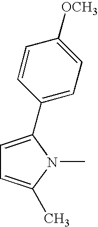 | 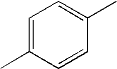 | 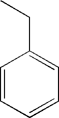 | COOEt | O | 2 | 1 | 1 | 512 | 96 |
| 42. | 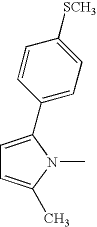 | 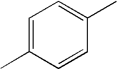 | 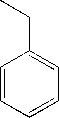 | COOEt | O | 2 | 1 | 1 | 528 | 28 |
| 43. | 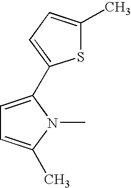 | 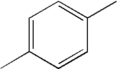 | 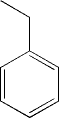 | COOEt | O | 2 | 1 | 1 | 502 | 10 |
| 44. | 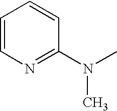 | 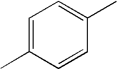 | 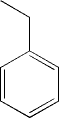 | COOEt | O | 2 | 1 | 1 | 433 | 94 |
| 45. | 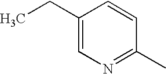 | 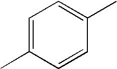 | 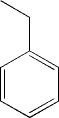 | COOEt | O | 2 | 1 | 1 | 432 | 61 |
| 46. | 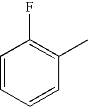 | 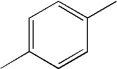 | 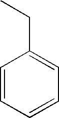 | COOEt | O | 1 | 1 | 1 | 407 | 94 |
| 47. | 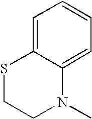 | 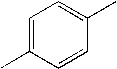 | 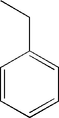 | COOEt | O | 2 | 1 | 1 | 476 | 100 |
| 48. | 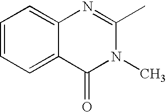 | 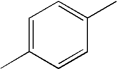 | 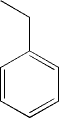 | COOEt | O | 1 | 1 | 1 | 471 | 98 |
| 49. | 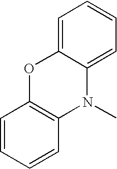 | 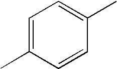 | 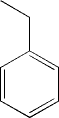 | COOEt | O | 2 | 1 | 1 | 508 | 49 |
| 50. | 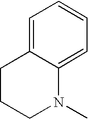 | 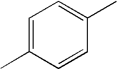 | 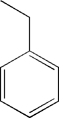 | COOEt | O | 2 | 1 | 1 | 458 | 92 |
| 51. | 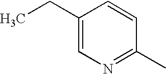 | 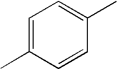 | 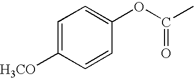 | COOEt | O | 2 | 1 | 1 | 492 | 71 |
| 52. | 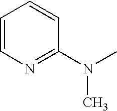 | 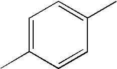 | 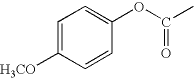 | COOEt | O | 2 | 1 | 1 | 493 | 88 |
| 53. | 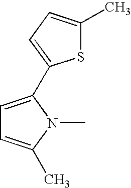 | 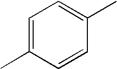 | 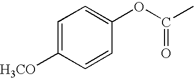 | COOEt | O | 2 | 1 | 1 | 562 | 82 |
| 54. | 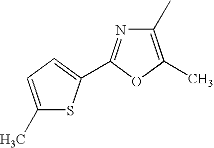 | 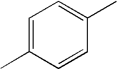 | 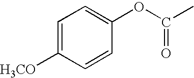 | COOEt | O | 2 | 1 | 1 | 564 | 37 |
| 55. | 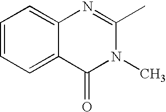 | 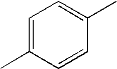 | 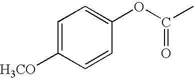 | COOEt | O | 1 | 1 | 1 | 531 | 59 |
| 56. | 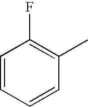 | 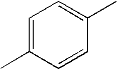 | 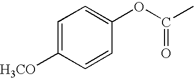 | COOEt | O | 1 | 1 | 1 | 467 | 94 |
| 57. | 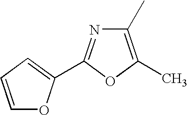 | 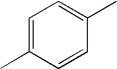 | 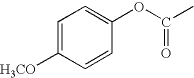 | COOEt | O | 2 | 1 | 1 | 534 | 58 |
| 58. | 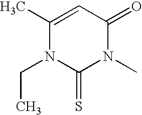 | 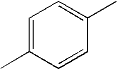 | 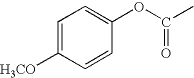 | COOEt | O | 2 | 1 | 1 | 555 | 73 |
| 59. | 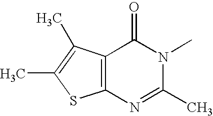 | 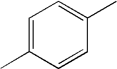 | 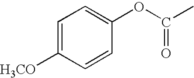 | COOEt | O | 2 | 1 | 1 | 579 | 44 |
| 60. | 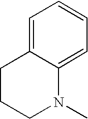 | 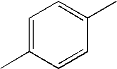 | 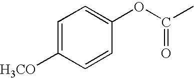 | COOEt | O | 2 | 1 | 1 | 518 | 80 |
| 61. | 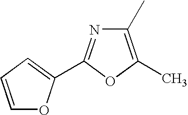 | 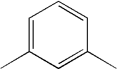 | 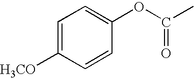 | COOEt | O | 2 | 1 | 1 | 534 | 20 |
| 62. | 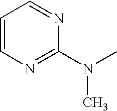 | 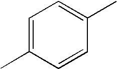 | 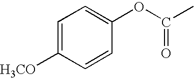 | COOEt | O | 2 | 1 | 1 | 494 | 78 |
| 63. | 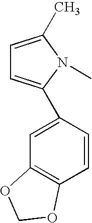 | 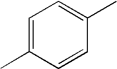 | 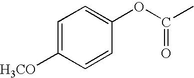 | COOEt | O | 2 | 1 | 1 | 586 | 100 |
| | ¹H: 1.26 (3H, t, J=6.15 Hz), 2.04-2.30 (2H, m), 2.36 (3H, s), 3.79 (3H, s), 3.93-3.97 (4H, m), | | | | | | | | | |
| | 4.17-4.26 (4H, m), 5.93 (1H, d, J=3.15 Hz), 5.99 (3H, s), 6.04 (1H, d, J=3.3 Hz), 6.66-6.71 | | | | | | | | | |
| | (2H, m), 6.84-6.87 (4H, m), 7.05 (2H, d, J=8.73 Hz), 7.17 (2H, d, J=6.48 Hz). | | | | | | | | | |
| 64. | 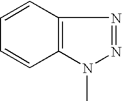 | 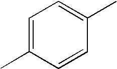 | 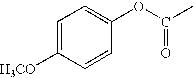 | COOEt | O | 2 | 1 | 1 | 504 | 50 |
| | ¹H: 1.22-1.25 (3H, m), 3.79 (3H, s), 3.96 (2H, s), 4.13-4.18 (2H, m), 4.54-4.67 (4H, m), 5.11 | | | | | | | | | |
| | (2H, t, J=5.50 Hz), 6.85-6.90 (4H, m), 7.01 (2H, d, J=7.2 Hz), 7.20 (2H, d, J=8.59 Hz), | | | | | | | | | |
| | 7.38-7.41 (2H, m), 7.86-7.89 (2H, m). | | | | | | | | | |
| 65. | 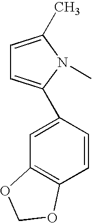 | 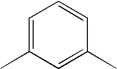 | 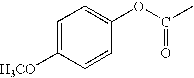 | COOEt | O | 2 | 1 | 1 | 586 | 22 |
| | ¹H: 1.30 (3H, t, J=7.3 Hz), 2.36 (3H, s), 3.79 (3H, s), 3.93-3.98 (4H, m), 4.10-4.29 (4H, m), | | | | | | | | | |
| | 4.56-4.66 (2H, m), 5.93 (1H, d, J=2.85 Hz), 5.98 (2H, s), 6.03 (1H, d, J=3.36 Hz), 6.62-6.68 | | | | | | | | | |
| | (2H, m), 6.84-6.87 (6H, m), 7.01-7.25 (3H, m). | | | | | | | | | |
| 66. | 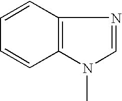 | 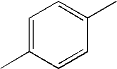 | 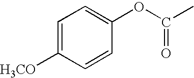 | COOEt | O | 2 | 1 | 1 | 503 | 39 |
| | ¹H: 1.20-1.28 (3H, m), 3.78 (3H, s), 3.96 (2H, s), 4.15-4.18 (2H, m), 4.30-4.32 (2H, m), 4.54 | | | | | | | | | |
| | (1H, s), 4.56-4.59 (2H, m), 4.64 (1H, s), 6.81 (2H, d, J=8.49 Hz), 6.85 (2H, d, J=9.15 Hz), 7.02 | | | | | | | | | |
| | (2H, d, J=8.97 Hz), 7.22 (2H, d, J=8.58 Hz, 7.26-7.81 (4H, m), 8.06 (1H, s). | | | | | | | | | |
| 67. | 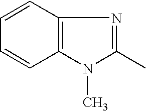 | 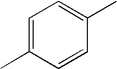 | 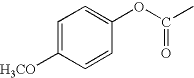 | COOEt | O | 1 | 1 | 1 | 503 | 43 |
| | ¹H: 1.98 (3H, t, J=7.38 Hz), 3.79 (3H, s), 3.89 (3H, s), 3.97 (2H, s), 4.11-4.18 (2H, m), 4.55 | | | | | | | | | |
| | (1H, s), 4.66 (1H, s), 5.42 (2H, s), 6.86 (2H, d, J=9.03 Hz), 7.02 (2H, d, J=8.28 Hz), 7.06 (2H, | | | | | | | | | |
| | d, J=8.75 Hz), 7.23-7.26 (2H, m), 7.28-7.77 (3H, m), 7.78-7.80 (1H, m). | | | | | | | | | |
| 68. | 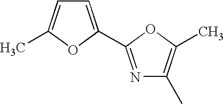 | 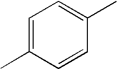 | 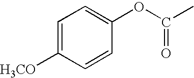 | COOEt | O | 2 | 1 | 1 | 548 | 20 |
| 69. | 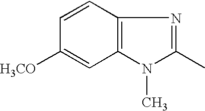 | 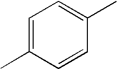 | 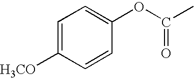 | COOEt | O | 1 | 1 | 1 | 533 | 30 |
| 70. | 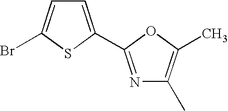 | 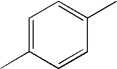 | 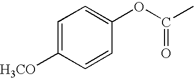 | COOEt | O | 2 | 1 | 1 | 629 | 36 |
| 71. | 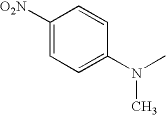 | 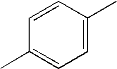 | 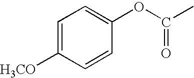 | COOEt | O | 2 | 1 | 1 | 537 | 33 |
| | ¹H: 1.24 (3H, t, J=7.11 Hz), 3.19 (3H, s), 3.79 (3H, s), 3.88 (2H, t, J=5.44 Hz), 3.98 (2H, s), | | | | | | | | | |
| | 4.15-4.19 (4H, m), 4.55 (1H, s), 4.66 (1H, s), 6.69 (2H, d, J=9.33 Hz), 6.81-6.88 (4H, m), | | | | | | | | | |
| | 7.04 (2H, d, J=8.79 Hz), 7.22-7.26 (2H, m), 8.13 (2H, d, J=9.39 Hz). | | | | | | | | | |
| 72. | 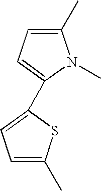 | 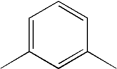 | 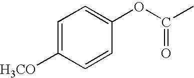 | COOEt | O | 2 | 1 | 1 | 562 | 30 |
| | ¹H: 1.25 (3H, t, J=6.67 Hz), 2.36 (3H, s), 2.48 (3H, s), 3.79 (3H, s), 3.99 (2H, s), 4.08 (2H, t, | | | | | | | | | |
| | J=6.26 Hz), 4.15-4.22 (2H, q, J=7.10 Hz), 4.34 (2H, t, J=6.25 Hz), 4.57 (1H, s), 4.67 (1H, s), | | | | | | | | | |
| | 5.91 (1H, d, J=3.37 Hz), 6.18 (1H, d, J=3.41 Hz), 6.72 (2H, d, J=9 Hz), 6.81-6.89 (4H, m), | | | | | | | | | |
| | 7.02 (2H, d, J=8.96 Hz), 7.18-7.25 (1H, m). | | | | | | | | | |
| 73. | 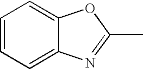 | 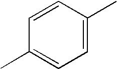 | 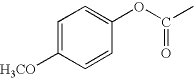 | COOEt | O | 1 | 1 | 1 | 490 | 26 |
| | ¹H: 1.23 (3H, t, J=6.67 Hz), 3.79 (3H, s), 3.99 (2H, s), 4.14-4.20 (2H, q, J=7.09 Hz), 4.57 (1H, | | | | | | | | | |
| | s), 4.67 (1H, s), 5.32 (2H, s), 6.87 (2H, d, J=6.84 Hz), 7.01-7.08 (4H, m), 7.27 (2H, d, J=6.93 | | | | | | | | | |
| | Hz), 7.37 (2H, m), 7.53-7.57 (1H, m), 7.75-7.78 (1H, m). | | | | | | | | | |
| 74. | 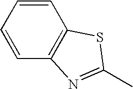 | 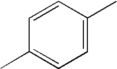 | 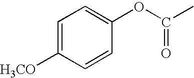 | COOEt | O | 1 | 1 | 1 | 506 | 78 |
| | ¹H: 0.8 (3H, m), 3.79 (3H, s), 3.99 (2H, S), 4.14-4.20 (2H, m), 4.57 (1H, s), 4.67 (1H, s), | | | | | | | | | |
| | 5.5 (2H, s), 6.85-6.88 (2H, m), 7.01-7.05 (4H, m), 7.26-7.28, (2H, m), 7.41 (1H, t, J=7.3 Hz), | | | | | | | | | |
| | 7.5 (1H, m), 7.9 (1H, d, J=8.0 Hz), 8.0 (1H, d, J=8.0 Hz). | | | | | | | | | |
| 75. | 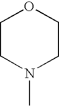 | 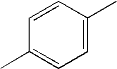 | 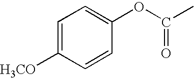 | COOEt | O | 2 | 1 | 1 | 472 | 41 |
| | ¹H: 1.25 (3H, t, J=7.1 Hz), 2.59 (4H, d, J=4.0 Hz), 2.79-2.83 (2H, m), 3.74 (4H, t, J=4.5 Hz), | | | | | | | | | |
| | 3.79 (3H, s), 3.99 (2H, d, J=3.2 Hz), 4.09-4.67 (4H, m), 4.56 (1H, s), 4.67 (1H, s), 6.86-6.91 | | | | | | | | | |
| | (4H, m), 7.01-7.07 (2H, m), 7.22-7.26 (2H, m). | | | | | | | | | |
| 76. | 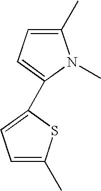 | 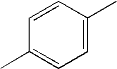 | 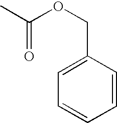 | COOEt | O | 2 | 1 | 1 | 546 | 83 |
| | ¹H: 1.15 (3H, t, J=7.12 Hz), 2.35 (3H, s), 2.48 (3H, s), 3.82 (1H, s), 3.91 (1H, s), 4.03-4.17 | | | | | | | | | |
| | (4H, m), 4.33 (2H, t, J=6.42 Hz), 4.50-4.51 (2H, m), 5.17-5.20 (2H, m), 5.91 (1H, d, J=3.18 | | | | | | | | | |
| | Hz), 6.17 (1H, d, J=3.42 Hz), 6.68-6.74 (3H, m), 6.80 (1H, d, J=3.42 Hz), 7.05 (1H, d, J=8.4 | | | | | | | | | |
| | Hz), 7.14 (1H, d, J=8.46 Hz), 7.330-7.336 (5H, m). | | | | | | | | | |
| 77. | 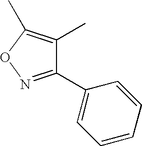 | 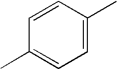 | 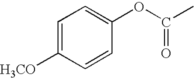 | COOEt | O | 2 | 1 | 1 | 544 | 25 |
| 78. | 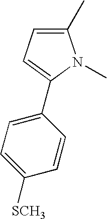 | 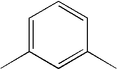 | 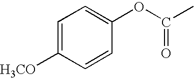 | COOEt | O | 2 | 1 | 1 | 588 | 34 |

### Preparation 9

### {Benzyl-[3-(2-indol-1-yl-ethoxy)-benzyl]-amino}-acetic acid.

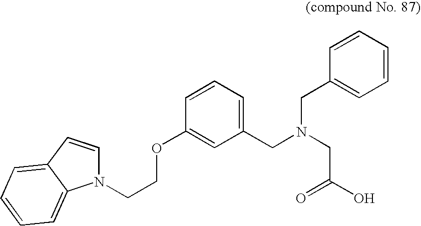 To a solution of ethyl-{benzyl-[3-(2-indol-1-yl-ethoxy)-benzyl]-amino}-acetate (compound No.17) (1.0 g) in ethanol (12 mL) was added a solution of sodium hydroxide (130 mg) in water (4 mL) and the reaction mixture was stirred at 30° C. for 16 hours. The solvent was evaporated under reduced pressure. Water (50 mL) was added to the residue, acidified with 1N HCl to pH 6 and extracted with ethyl acetate (4×30 mL). The combined organic extract was washed with water (50 mL), brine solution (50 mL), dried over sodium sulphate and evaporated under reduced pressure. The crude product was chromatographed (flash) over silica gel using 3.5% methanol in chloroform as an eluent to obtain 260 mg of pure product.

### Preparation 10

### [{4-[2-(2,3-Dihydro-benzo[1,4]oxazin-4-yl)-ethoxy]-benzyl}-(4-methoxy-phenoxycarbonyl)-amino]-acetic acid

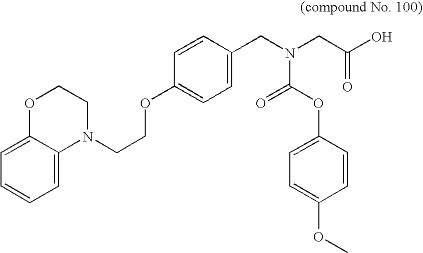

To a solution of ethyl-[{4-[2-(2,3-dihydro-benzo[1,4]oxazin-4-yl)-ethoxy]-benzyl}- (4-methoxy-phenoxyc.arbonyl)-amino]-acetate (compound No.32) (1.18 g) in a mixture of tetrahydrofuran (4.5 mL) and methanol (1.5 mL) was added a solution of LiOH.H₂0 (196 mg) in water (1.5 mL) and the reaction mixture was stirred at ambient temperature for 30 minutes. The solvent was evaporated under reduced pressure, water (50 mL) was added to the residue, acidified with iN HC1 to pH .6 and extracted with ethyl acetate (3×50 mL). The combined organic extract was washed with water (50 mL), brine solution (50 mL), dried over sodium sulphate and evaporated under reduced pressure. The crude product was chromatographed (flash) over silica-gel using 1% methanol in chloroform as an eluent to obtain 360 mg of pure product.

In like manner compounds in table 3 were prepared by a procedure similar to that described in preparation 9 & 10.

TABLE 3

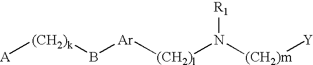

| S. | | | | | | | | | Mol. | % |
|---|---|---|---|---|---|---|---|---|---|---|
| No | A | Ar | R₁ | Y | B | k | 1 | m | Wt. | Yield |
| 79. | 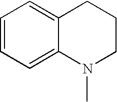 | 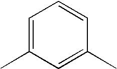 | 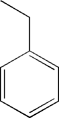 | COOH | O | 2 | 1 | 1 | 430 | 24 |
| | ¹H: 1.94 (2H, t, J=6.93 Hz), 2.75 (2H, t, J=6.17 Hz), 3.26 (2H, s), 3.42 (2H, t, J=5.54 Hz), | | | | | | | | | |
| | 3.69 (4H, m), 3.77 (2H, s), 4.13 (2H, t, J=6.10 Hz), 6.59 (2H, m), 6.82 (2H, d, J=5.73 Hz), | | | | | | | | | |
| | 6.88 (1H, d, J=7.5 Hz), 6.94 (1H, d, J=6.96 Hz), 7.05 (1H, t, J=7.56 Hz), 7.22 (6H, | | | | | | | | | |
| | complex). | | | | | | | | | |
| 80. | 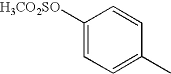 | 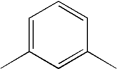 | 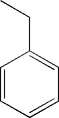 | COOH | O | 2 | 1 | 1 | 469 | 42 |
| | ¹H: 3.0 (2H, t, J=6.7 Hz), 3.1 (3H, s), 3.2 (2H, s), 3.79 (2H, s), 3.83 (2H, s), 4.1 (2H, t, J=6.7 | | | | | | | | | |
| | Hz), 6.9 (3H, m), 7.2 (4H, m), 7.6 (6H, m). | | | | | | | | | |
| 81. | 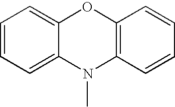 | 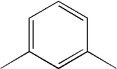 | 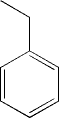 | COOH | O | 2 | 1 | 1 | 480 | 62 |
| | ¹H: 3.24 (2H, s), 3.77 (2H, s), 3.78 (2H, s), 3.98 (2H, t, J=6.13 Hz), 4.20 (2H, t, J=6.06 Hz), | | | | | | | | | |
| | 6.64 (5H, m), 6.79 (3H, dd, J=15.52 & 7.06 Hz), 6.97 (2H, d, J=6.24 Hz), 7.25 (4H, m), 7.36 | | | | | | | | | |
| | (2H, d, J=6.93 Hz), 7.58 (1H, s). | | | | | | | | | |
| 82. | 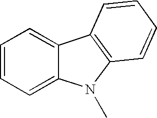 | 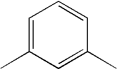 | 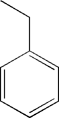 | COOH | O | 2 | 1 | 1 | 464 | 85 |
| | DMSO-D₆, ¹H: 3.10 (2H, s), 3.62 (2H, s), 3.65 (2H, s), 4.33 (2H, t, J=5.10 Hz), 4.79 (2H, t, | | | | | | | | | |
| | J=5.07 Hz), 6.67 (1H, dd, J=1.87 & 8.085 Hz), 6.78 (1H, s), 6.84 (1H, d, J=7.43 Hz), | | | | | | | | | |
| | 7.11-7.23 (4H, m), 7.11-7.23 (4H, m), 7.44 (2H, t, J=7.18 Hz), 7.68 (2H, d, J=8.25 Hz). | | | | | | | | | |
| | 8.12 (2H, d, J=7.68 Hz). | | | | | | | | | |
| 83. | 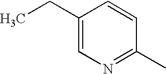 | 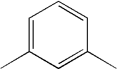 | 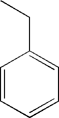 | COOH | O | 2 | 1 | 1 | 404 | 64 |
| | ¹H: 1.24 (3H, t, J=15.3 Hz), 2.63 (2H, q, J=15.18 & 7.56 Hz), 3.22 (2H, t, J=6.91 Hz), 3.27 | | | | | | | | | |
| | (2H, s), 3.74 (2H, s), 3.81 (2H, s), 4.34 (2H, t, J=6.93 Hz), 6.83 (2H, m), 6.95 (1H, s), 7.21 | | | | | | | | | |
| | (2H, dd, J=7.86 & 2.25 Hz), 7.33 (5H, m), 7.5 (1H, d, J=6.82 & 2.13 Hz), 8.4 (1H, d, J=1.62 | | | | | | | | | |
| | Hz). | | | | | | | | | |
| 84. | 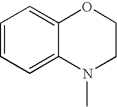 | 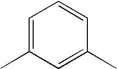 | 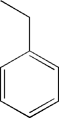 | COOH | O | 2 | 1 | 1 | 432 | 86 |
| | ¹H: 3.28 (2H, s), 3.5 (2H, t, J=4.5 Hz), 3.69 (2H, t, J=5.6 Hz), 3.75 (2H, s), 3.79 (2H, s), 4.1 | | | | | | | | | |
| | (2H, t, J=5.6 Hz), 4.2 (2H, t, J=4.3 Hz), 6.6 (1H, m), 6.7 (1H, d, J=7.0 Hz), 6.77-6.83 (4H, | | | | | | | | | |
| | m), 6.9 (1H, d, J=7.58 Hz), 7.2-7.35 (6H, m). | | | | | | | | | |
| 85. | 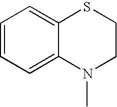 | 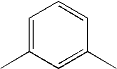 | 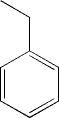 | COOH | O | 2 | 1 | 1 | 448 | 92 |
| | ¹H: 3.0 (2H, t, J=4.66 Hz), 3.3 (2H, s), 3.7 (4H, m), 3.79 (2H, s), 3.83 (2H, s), 4.1 (2H, t, | | | | | | | | | |
| | J=5.58 Hz), 6.6 (1H, t, J=7.3 Hz), 6.7 (1H, d, J=8.2 Hz), 6.8-7.0 (5H, m), 7.2-7.32 (6H, m). | | | | | | | | | |
| 86. | 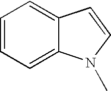 | 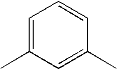 | 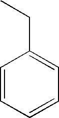 | COOH | O | 2 | 1 | 1 | 414 | 58 |
| | ¹H: 3.25 (2H, s), 3.7 (2H, s), 3.75 (2H, s), 4.26 (2H, t, J=5.56 Hz), 4.52 (2H, t, J=5.5 Hz), 6.5 | | | | | | | | | |
| | (1H, d, J=3.07 Hz), 6.7 (2H, d, J=7.45 Hz), 6.8 (1H, d, J=7.43 Hz), 7.1 (1H, t, J=7.03 Hz), | | | | | | | | | |
| | 7.20-7.38 (7H, m), 7.4 (2H, d, J=8.26 Hz), 7.6 (1H, d, J=7.8 Hz). | | | | | | | | | |
| 87. | 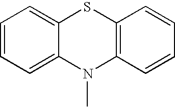 | 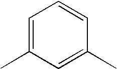 | 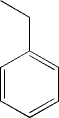 | COOH | O | 3 | 1 | 1 | 510 | ′94 |
| | ¹H: 2.26 (2H, m), 3.24 (2H, s), 3.75 (2H, s), 3.79 (2H, s), 4.0 (4H, t, J=6.07 Hz), 6.7 (1H, d, | | | | | | | | | |
| | J=8.3 Hz), 6.88-6.96 (6H, m), 7.10-7.20 (5H, m), 7.22-7.32 (3H, m), 7.38 (2H, d, J=7.19 | | | | | | | | | |
| | Hz). | | | | | | | | | |
| 88. | 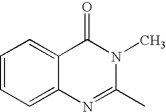 | 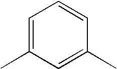 | 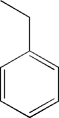 | COOH | O | 1 | 1 | 1 | 443 | 88 |
| | ¹H: 3.2 (2H, s), 3.7 (3H, s), 4.1 (2H, s), 4.2 (2H, s), 5.3 (2H, s), 7.1 (2H, t, J=9.6 Hz), 7.2 (1H, | | | | | | | | | |
| | s), 7.3-7.4 (6H, m), 7.5 (1H, t, J=7.3 Hz), 7.69 (1H, d, J=8.1 Hz), 7.7 (1H, t, J=7.5 Hz), 8.2 | | | | | | | | | |
| | (1H, d, J=7.9 Hz). | | | | | | | | | |
| 89. | 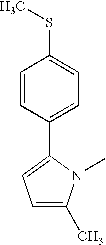 | 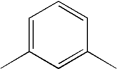 | 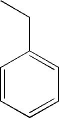 | COOH | O | 2 | 1 | 1 | 500 | 100 |
| | ¹H: 2.3 (3H, s), 2.5 (3H, s), 3.2 (2H, s), 3.7 (2H, s), 3.8 (2H, s), 3.9 (2H, t, J=6.4 Hz), 4.3 | | | | | | | | | |
| | (2H, t, J=6.4 Hz), 5.9 (1H, d, J=3.2 Hz), 6.1 (1H, d, J=3.4 Hz), 6.6 (1H, dd, J=8.2 & 2.1 Hz), | | | | | | | | | |
| | 6.7 (1H, s), 6.9 (1H, d, J=7.7 Hz), 7.2 (2H, t, J=7.9 Hz), 7.3 (3H, m), 7.35 (5H, m). | | | | | | | | | |
| 90. | 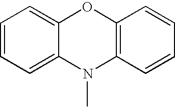 | 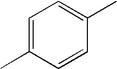 | 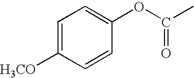 | COOH | O | 2 | 1 | 1 | 540 | 71 |
| | ¹H: 3.78 (3H, s), 3.9 (2H, m), 4.03 (2H, d, J=4.26 Hz), 4.19 (2H, m), 4.57 (1H, s), 4.67 (1H, | | | | | | | | | |
| | s), 6.61-6.7 (6H, m), 6.7-6.0 (6H, m), 7.04 (2H, t, J=9.3 Hz), 7.2 (2H, d, J=10.26 Hz). | | | | | | | | | |
| 91. | 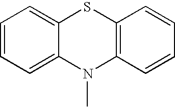 | 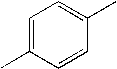 | 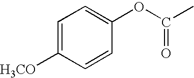 | COOH | O | 2 | 1 | 1 | 556 | 59 |
| | ¹H: 3.78 (3H, s), 4.04 (2H, d, J=4.86 Hz), 4.31 (4H, s), 4.56 (1H, s), 4.6 (1H, s), 6.85-6.91 | | | | | | | | | |
| | (4H, m), 6.96 (3H, d, J=7.2 Hz), 7.04 (3H, t, J=8.7 Hz), 7.13 (3H, d, J=7.57 Hz), 7.18-7.23 | | | | | | | | | |
| | (3H, m). | | | | | | | | | |
| 92. | 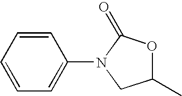 | 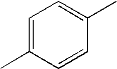 | 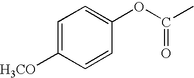 | COOH | O | 1 | 1 | 1 | 506 | 62 |
| | ¹H: 3.78 (3H, s), 4.08 (3H, m), 4.16-4.25 (3H, m), 4.57 (1H, s), 4.67 (1H,s), 4.98 (1H, m), | | | | | | | | | |
| | 6.84-6.93 (4H, m), 7.04 (2H, t, J=8.64 Hz), 7.18 (1H, t, J=7.4 Hz), 7.27 (2H, m), 7.39 (2H, t, | | | | | | | | | |
| | J=7.9 Hz), 7.5 (2H, d, J=7.9 Hz). | | | | | | | | | |
| 93. | 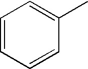 | 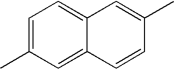 | 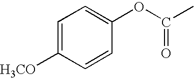 | COOH | O | 1 | 1 | 1 | 471 | 86 |
| | ¹H: 3.79 (3H, s), 4.09 (2H, s), 4.7 (1H, s), 4.87 (1H, s), 5.18 (2H, s), 6.87 (2H, d, J=8.7 Hz), | | | | | | | | | |
| | 7.08 (2H, t, J=8.9 Hz), 7.24 (2H, m), 7.32-7.43 (4H, m), 7.50 (2H, d, J=7.3 Hz), 7.63 (1H, s), | | | | | | | | | |
| | 7.73 (2H, t, J=8.0 Hz). | | | | | | | | | |
| 94. | 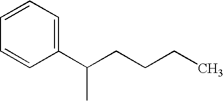 | 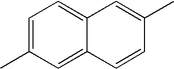 | 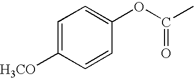 | COOH | O | 0 | 1 | 1 | 527 | 70 |
| | ¹H: 0.9 (3H, t, J=6.8 Hz), 1.25-1.5 (4H, m), 1.89 (1H, m), 2.04 (1H, m), 3.7 (3H, s), 4.03 (2H, | | | | | | | | | |
| | s), 4.71 (1H, s), 4.8 (1H, s), 5.25 (1H, t, J=5.8 Hz), 6.85 (2H, d, J=8.5 Hz), 7.0-7.08 (3H, m), | | | | | | | | | |
| | 7.2 (2H, m), 7.32 (3H, m), 7.4 (2H, m), 7.58 (2H, m), 7.69 (1H, d, J=8.91 Hz). | | | | | | | | | |
| 95. | 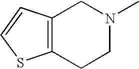 | 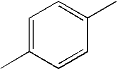 | 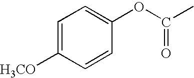 | COOH | O | 2 | 1 | 1 | 496 | 50 |
| | ¹H: 3.03 (2H, t, J=5.3 Hz), 3.24 (4H, m), 3.7 (3H, s), 3.86 (2H, d, J=6.84 Hz), 4.01 (2H, s), | | | | | | | | | |
| | 4.25 (2H, t, J=5.3 Hz), 4.52 (1H, s), 4.6 (1H, s), 6.74 (1H, d, J=5.14 Hz), 6.81-6.87 (4H, m), | | | | | | | | | |
| | 7.0 (2H, m), 7.14 (1H, d, J=5.14 Hz), 7.18 (2H, d, J=7.9 Hz). | | | | | | | | | |
| 96. | 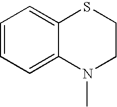 | 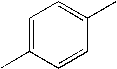 | 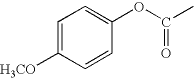 | COOH | O | 2 | 1 | 1 | 508 | 52 |
| | ¹H: 3.04 (2H, t, J=5.0 Hz), 3.74-3.79 (7H, complex), 4.04 (2H, d, J=4.98 Hz), 4.17 (2H, t, | | | | | | | | | |
| | J=4.66 Hz), 4.56 (1H, s), 4.67 (1H, s), 6.62 (1H, t, J=7.33 Hz), 6.72 (1H, d, J=8.16 Hz), 6.87 | | | | | | | | | |
| | (4H, t, J=6.21 Hz), 6.96-7.07 (4H, m), 7.21 (2H, m). | | | | | | | | | |
| 97. | 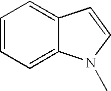 | 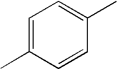 | 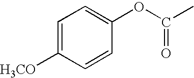 | COOH | O | 2 | 1 | 1 | 474 | 15 |
| | ¹H: 3.78 (3H, s), 4.0 (2H, d, J=3.24 Hz), 4.27 (2H, t), 4.54 (3H, m), 4.64 (1H, s), 6.51 (1H, d, | | | | | | | | | |
| | J=2.76 Hz), 6.52-6.88 (4H, m), 7.03 (2H, t, J=8.8 Hz), 7.1 (1H, t, J=7.42 Hz), 7.21 (4H, t, | | | | | | | | | |
| | J=5.86 Hz), 7.41 (1H, d, J=8.16 Hz), 7.63 (1H, d, J=7.8 Hz). | | | | | | | | | |
| 98. | 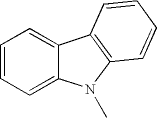 | 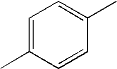 | 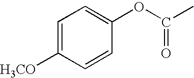 | COOH | O | 2 | 1 | 1 | 524 | 48 |
| | ¹H: 3.8 (3H, s), 4.0 (2H, s), 4.3 (2H, m), 4.5 (1H, s), 4.6 (1H, s), 4.7 (2H, t, J=4.8 Hz), 6.8 | | | | | | | | | |
| | (2H, t, J=7.4 Hz), 6.9 (2H, m), 7.0 (2H, t, J=8.6 Hz), 7.1 (2H, d, J=8.2 Hz), 7.2 (2H, m), 7.5 | | | | | | | | | |
| | (4H, m) 8.1 (2H, d, J=7.8 Hz). | | | | | | | | | |
| 99. | 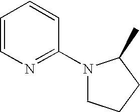 | 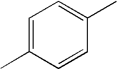 | 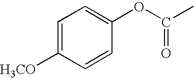 | COOH | O | 1 | 1 | 1 | 491 | 48 |
| | ¹H: 2.1 (2H, m), 2.2 (2H, m), 3.3 (2H, m), 3.8 (3H, s), 3.85 (1H, m), 3.9 (2H, d, J=10.5 Hz) | | | | | | | | | |
| | 4.2 (1H, m), 4.4 (1H, m), 4.5 (1H, s), 4.6 (1H, s), 6.5 (1H, d, J=8.5 Hz), 6.6 (1H, m), 6.8 | | | | | | | | | |
| | (4H, m), 7.0 (2H, m), 7.2 (2H, d, J=8.5 Hz), 7.5 (1H, m), 8.1 (1H, d, J=4.3 Hz). | | | | | | | | | |
| 100. | 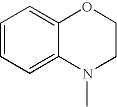 | 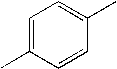 | 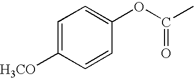 | COOH | O | 2 | 1 | 1 | 492 | 31 |
| | ¹H: 3.51 (2H, s), 3.70 (2H, d, J=5.39 Hz), 3.78 (3H, s), 4.0 (2H, d, J=5.4 Hz), 4.15-4.2 (4H, | | | | | | | | | |
| | m), 4.56 (1H, s), 4.66 (1H, s), 6.6 (1H, t, J=7.0 Hz), 6.7 (1H, d, J=7.9 Hz), 6.79-6.86 (6H, m), | | | | | | | | | |
| | 7.04 (2H, t, J=9.54 Hz), 7.2 (2H, d, J=8.52 Hz). | | | | | | | | | |
| 101. | 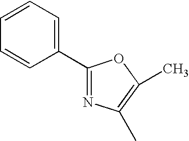 | 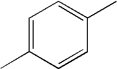 | 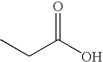 | COOH | O | 2 | 1 | 1 | 424 | 73 |
| | DMSO-d₆, ¹H: 3.35 (3H, s), 2.91 (2H, t, J=6.2 Hz), 3.35 (4H, s), 3.93 (2H, s), 4.2 (2H, t, | | | | | | | | | |
| | J=6.3 Hz), 6.86 (2H, d, J=8.2 Hz), 7.22 (2H, d, J=8.2 Hz), 7.35 (3H, m), 7.9 (2H, d, J=7.35 | | | | | | | | | |
| | Hz). | | | | | | | | | |
| 102. | 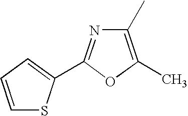 | 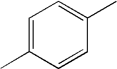 | 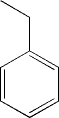 | COOH | O | 2 | 1 | 1 | 462 | 84 |
| | ¹H: 2.34 (3H, s), 2.95 (2H, t, J=6.51 Hz), 3.29 (2H, s), 3.81 (2H, s), 3.84 (2H, s), 4.20 (2H, t, | | | | | | | | | |
| | J=6.58 Hz), 6.86 (2H, d, J=8.49 Hz), 7.05-7.08 (1H, m), 7.21-7.36 (8H, m), 7.57-7.58 (1H, d, | | | | | | | | | |
| | J=3.12 Hz). | | | | | | | | | |
| 103. | 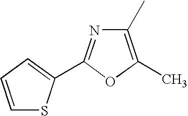 | 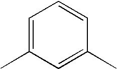 | 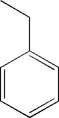 | COOH | O | 2 | 1 | 1 | 462 | 85 |
| | DMSO-d₆ ¹H: 2.30 (3H, s,), 2.88 (2H, t, J=6.28 Hz), 3.14 (2H, s), 3.68 (2H, s), 3.70 (s, 2H), | | | | | | | | | |
| | 4.15 (2H, t, J=6.40 Hz), 6.7-6.9 (3H, m), 7.06-7.3 (7H, m), 7.58-7.86 (2H, m). | | | | | | | | | |
| 104. | 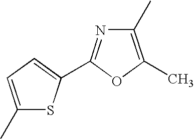 | 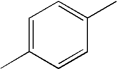 | 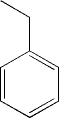 | COOH | O | 2 | 1 | 1 | 476 | 40 |
| | ¹H: 2.33 (3H, s), 2.50 (3H, s), 2.93 (2H, t, J=6.57 Hz), 3.26 (2H, s), 3.74 (2H, s), 3.78 (2H, s), | | | | | | | | | |
| | 4.20 (2H, t, J=6.63 Hz), 6.71 (1H, d, J=2.67 Hz), 6.85 (2H, d, J=8.49 Hz), 7.18 (2H, d, | | | | | | | | | |
| | J=8.49 Hz), 7.28-7.37 (6H, m). | | | | | | | | | |
| 105. | 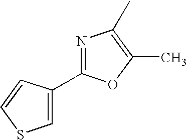 | 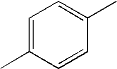 | 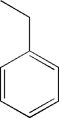 | COOH | O | 2 | 1 | 1 | 462 | 45 |
| | ¹H: 2.34 (3H, s), 2.96 (2H, t, J=6.58 Hz), 3.26 (2H, s), 3.75 (2H, s), 3.79 (2H, s), 4.21 (2H, | | | | | | | | | |
| | t, J=6.62 Hz), 6.86 (2H, d, J=8.54 Hz), 7.18 (2H, d, J=8.52 Hz), 7.28-7.37 (6H, m), 7.55-7.57 | | | | | | | | | |
| | (1H, m), 7.83-7.84 (1H, m). | | | | | | | | | |
| 106. | 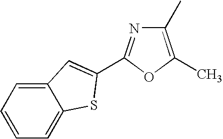 | 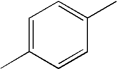 | 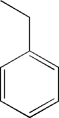 | COOH | O | 2 | 1 | 1 | 512 | 25 |
| | ¹H: 2.39 (3H, s), 2.98 (2H, t, J=6.48 Hz), 3.25 (s, 2H), 3.72 (2H, s), 3.77 (2H, s), 4.24 (2H, t, | | | | | | | | | |
| | J=6.55 Hz), 6.87 (2H, d, J=8.53 Hz) 7.18 (2H, d, J=8.53 Hz), 7.30-7.40 (7H, m), 7.79-7.85 | | | | | | | | | |
| | (3H, m). | | | | | | | | | |
| 107. | 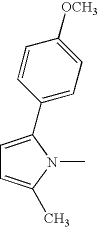 | 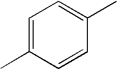 | 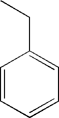 | COOH | O | 2 | 1 | 1 | 484 | 46 |
| | ¹H: 2.36 (3H, s), 3.25 (2H, s), 3.74 (3H, s), 3.79 (2H, s), 3.83 (2H, s), 3.92 (2H, t, J=6.51 | | | | | | | | | |
| | Hz), 4.25 (2H, t, J=6.51 Hz), 5.95 (1H, d, J=2.79 Hz), 6.05 (1H, d, J=3.3 Hz), 6.67 (2H, d, | | | | | | | | | |
| | J=8.55 Hz), 6.95 (2H, d, J=8.73 Hz), 7.16 (2H, d, J=8.55 Hz), 7.25-7.3 (3H, m), 7.31-7.33 | | | | | | | | | |
| | (4H, m). | | | | | | | | | |
| 108. | 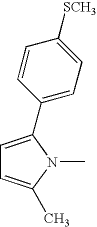 | 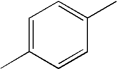 | 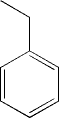 | COOH | O | 2 | 1 | 1 | 500 | 53 |
| | ¹H: 2.36 (3H, s), 2.5 (3H, s), 3.26 (2H, s), 3.76 (2H, s), 3.81 (2H, s), 3.92 (2H, t, J=6.46 Hz), | | | | | | | | | |
| | 4.27 (2H, t, J=6.46 Hz), 5.96 (1H d, J=3.15 Hz), 6.09 (1H, d, J=3.36 Hz) 6.65 (2H, d, J=8.55 | | | | | | | | | |
| | Hz), 7.17 (2H, d, J=8.55 Hz), 7.29-7.37 (9H, m). | | | | | | | | | |
| 109. | 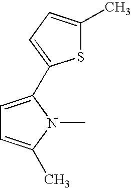 | 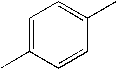 | 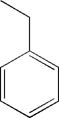 | COOH | O | 2 | 1 | 1 | 474 | 61 |
| | ¹H: 2.35 (3H, s), 2.48 (3H, s), 3.24 (2H, s), 3.69 (2H, s), 3.74 (2H, s), 4.07 (2H, t, J=6.2 Hz), | | | | | | | | | |
| | 4.34 (2H, t, J=6.4 Hz) 5.9 (1H, d, J=3.0 Hz), 6.18 (1H, d, J=3.4 Hz), 6.70 (1H, d, J=2.3 Hz), | | | | | | | | | |
| | 6.77 (2H, d, J=8.5 Hz), 6.8 (1H, d, J=3.4 Hz), 7.14 (2H, d, J=8.5 Hz), 7.29-7.38 (5H, m). | | | | | | | | | |
| 110. | 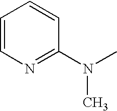 | 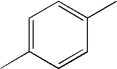 | 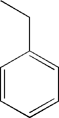 | COOH | O | 2 | 1 | 1 | 405 | 61 |
| | ¹H: 3.14 (3H, s), 3.25 (2H, s), 3.72 (2H, s), 3.77 (2H, s), 3.98 (2H, t, J=5.58 Hz), 4.18 (2H, t, | | | | | | | | | |
| | J=5.55 Hz), 6.5 (2H, d, J=8.55 Hz), 6.89 (2H, d, J=8.31 Hz), 7.26 (2H, d, J=8.37 Hz), 7.3-7.33 | | | | | | | | | |
| | (4H, m), 7.42-7.47 (2H, m), 8.14-8.15 (1H, m). | | | | | | | | | |
| 111. | 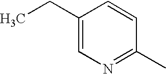 | 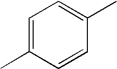 | 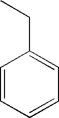 | COOH | O | 2 | 1 | 1 | 404 | 84 |
| | ¹H: 1.24 (3H, t, J=6.99 Hz), 2.64 (2H, q, J=7.9 Hz), 3.23 (2H, t, J=6.6 Hz), 3.28 (2H, s), 3.8 | | | | | | | | | |
| | (2H, s), 3.84 (2H, s), 3.99 (2H, t, J=6.61 Hz), 6.87 (2H, d, J=8.58 Hz), 7.18-7.29 (3H, m), | | | | | | | | | |
| | 7.29-7.33 (5H, m), 7.45-7.49 (1H, dd, J1=2.16 Hz, J2=7.92 Hz), 8.4 (1H, s). | | | | | | | | | |
| 112. | 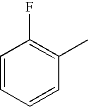 | 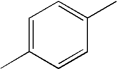 | 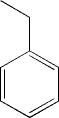 | COOH | O | 1 | 1 | 1 | 379 | 56 |
| | ¹H: 3.38 (2H, s), 3.76 (2H, s), 3.80 (2H, s), 5.12 (2H, s), 6.97 (2H, d, J=8.58 Hz), | | | | | | | | | |
| | 7.05-7.51 (11H, m) | | | | | | | | | |
| 113. | 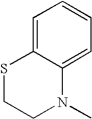 | 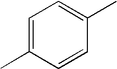 | 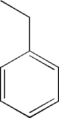 | COOH | O | 2 | 1 | 1 | 448 | 57 |
| | ¹H: 3.04 (2H, t, J=6.06 Hz), 3.25 (2H, s), 3.72 (2H, s), 3.76 (2H, s), 3.74-3.79 (4H, m), 4.16 | | | | | | | | | |
| | (2H, t, J=5.77 Hz), 6.59-6.61 (1H, m), 6.73 (1H, d, J=7.36 Hz), 6.88 (2H, d, J=8.556 Hz), | | | | | | | | | |
| | 6.98-7.04 (2H, m), 7.21 (2H, d, J=8.52 Hz), 7.27-7.37 (5H, m) | | | | | | | | | |
| 114. | 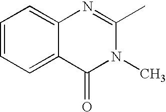 | 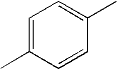 | 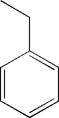 | COOH | O | 1 | 1 | 1 | 443 | 76 |
| | DMSO-d₆, ¹H: 3.14 (3H, s), 3.326 (2H, s), 3.66 (2H, s), 3.70 (2H, s), 5.24 (2H, s), 7.08 (2H, | | | | | | | | | |
| | d, J=8.58 Hz), 7.22-7.32 (7H, m), 7.55 (1H, t, J=7.52 Hz), 7.65 (1H, d, J=8.05 Hz), | | | | | | | | | |
| | 7.79-7.94 (1H, m),8.15 (1H, d, J=7.93 Hz). | | | | | | | | | |
| 115. | 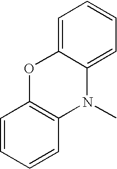 | 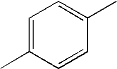 | 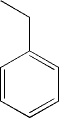 | COOH | O | 2 | 1 | 1 | 480 | 74 |
| | DMSO-d₆, ¹H: 3.1 (3H, s), 3.63 (2H, s), 3.68 (2H, s), 3.99 (2H, t, J=5.56 Hz) 4.17 (2H, t, | | | | | | | | | |
| | J=5.6 Hz), 6.61-6.63 (4H, m), 6.68-6.81 (2H, m), 6.88 (2H, d, J=8.5 Hz), 7.23 (3H, d, J=8.5 | | | | | | | | | |
| | Hz), 7.23-7.31 (5H, m). | | | | | | | | | |
| 116. | 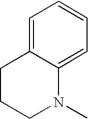 | 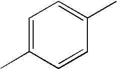 | 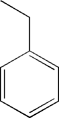 | COOH | O | 2 | 1 | 1 | 430 | 53 |
| | ¹H: 1.94 (2H, qui, J=6.10 Hz), 2.75 (2H, t, J=6.27 Hz), 3.28 (2H, s), 3.42 (2H, t, J=5.51 Hz), | | | | | | | | | |
| | 3.69 (2H, t, J=6.06 Hz), 3.79 (2H, s), 3.82 (2H, s), 4.13 (2H, t, J=6.09 Hz), 6.55-6.63 (2H, | | | | | | | | | |
| | m), 6.86 (2H, d, J=8.58 Hz), 6.94 (1H, d, J=7.11 Hz), 7.01-7.07 (1H, m), 7.22 (2H, d, | | | | | | | | | |
| | J=8.55 Hz), 7.30-7.38 (5H, m). | | | | | | | | | |
| 117. | 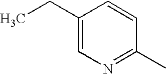 | 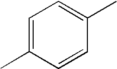 | 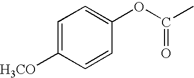 | COOH | O | 2 | 1 | 1 | 464 | 87 |
| | ¹H: 1.25 (3H, t, J=7.59 Hz), 2.68 (2H, q, J=7.57 Hz), 3.78 (2H, t, J=5.7 Hz), 3.78 (3H, s), | | | | | | | | | |
| | 3.9 (2H, s), 4.3 (2H, t, J=5.74 Hz) 4.58 (1H, s), 4.67 (1H, s), 6.83-6.88 (4H, m), 7.04-7.08 | | | | | | | | | |
| | (2H, m), 7.19-7.22 (2H, m), 7.27-7.3 (1H, m), 7.6 (1H, dd, J=2.16 & 7.92 Hz), 8.5 (1H, s) | | | | | | | | | |
| 118. | 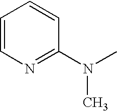 | 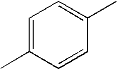 | 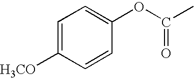 | COOH | O | 2 | 1 | 1 | 465 | 73 |
| | ¹H: 3.14 (3H, s), 3.75 (3H, s), 3.94-3.96 (4H, m), 4.14 (2H, s), 4.5 (1H, s), 4.6 (1H, s), 6.56-6.6 | | | | | | | | | |
| | (2H, m), 6.77-6.84 (4H, m), 7.03 (2H, d, J=8.9 Hz), 7.19 (2H, d, J=8.2 Hz), 7.48-7.53 | | | | | | | | | |
| | (1H, m), 8.15 (1H, s) | | | | | | | | | |
| 119. | 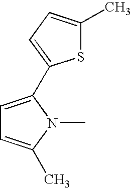 | 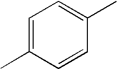 | 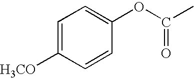 | COOH | O | 2 | 1 | 1 | 534 | 96 |
| | ¹H: 2.35 (3H, s), 2.48 (3H, s), 3.78 (3H, s), 4.03 (2H, t, J=5.83 Hz), 4.07 (2H, s), 4.34 (2H, t, | | | | | | | | | |
| | J=5.34 Hz), 4.54 (1H, s), 4.64 (1H, s), 5.91 (1H, d, J=2.97 Hz), 6.18 (1H, d, J=3.3 Hz), 6.69 | | | | | | | | | |
| | (1H, d, J=2.3 Hz), 6.74-6.79 (2H, m), 6.82 (1H, d, J=3.27 Hz), 6.84-6.88 (2H, m), 7.00-7.06 | | | | | | | | | |
| | (2H, m), 7.20 (2H, d, J=8.07 Hz). | | | | | | | | | |
| 120. | 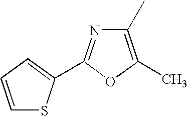 | 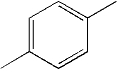 | 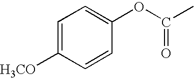 | COOH | O | 2 | 1 | 1 | 522 | 84 |
| | ¹H: 2.35 (3H, s), 2.96 (2H, t, J=6.48 Hz), 3.77 (3H, s), 4.02 (2H, s), 4.18-4.21 (2H, m), 4.55 | | | | | | | | | |
| | (1H, s), 4.64 (1H, s), 6.83-6.89 (4H, m), 7.01-7.08 (3H, m), 7.20 (2H, d, J=7.5 Hz), 7.35 | | | | | | | | | |
| | (1H, d, J=4.14 Hz), 7.59 (1H, d, J=3.54 Hz). | | | | | | | | | |
| 121. | 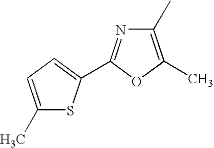 | 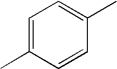 | 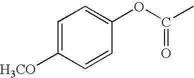 | COOH | O | 2 | 1 | 1 | 536 | 35 |
| | ¹H: 2.35 (3H, s)), 2.49 (3H, s), 2.96 (2H, s), 3.77 (3H, s), 4.0 (2H, s), 4.18 (2H, t, J=6.4 Hz), | | | | | | | | | |
| | 4.54 (1H, s), 4.64 (1H, s), 6.82 (1H, d, J=3.08 Hz), 6.72-6.87 (4H, m), 7.03 (2H, d, J=8.6 | | | | | | | | | |
| | Hz), 7.21 (2H, d, J=8.29 Hz), 7.41 (1H, d, J=3.58 Hz) | | | | | | | | | |
| 122. | 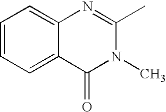 | 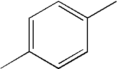 | 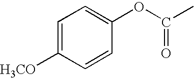 | COOH | O | 1 | 1 | 1 | 503 | 81 |
| | ¹H: 3.75 (3H, s), 3.78 (3H, s), 4.04 (2H, d, J=4.87 Hz), 4.57 (1H, s), 4.67 (1H, s), 5.20 (2H, | | | | | | | | | |
| | s), 6.83-6.87 (2H, dd, J1=5.14 Hz, J2=8.85 Hz), 7.00-7.07 (4H, m), 7.28 (2H, s), 7.49-7.54 | | | | | | | | | |
| | (1H, m), 7.71-7.79 (2H, m), 8.3 (1H, d, J=7.88 Hz) | | | | | | | | | |
| 123. | 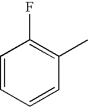 | 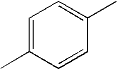 | 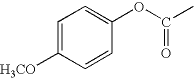 | COOH | O | 1 | 1 | 1 | 439 | 85 |
| | ¹H: 3.76 (3H, s), 4.05 (2H, d, J=10.68 Hz), 4.56 (1H, s), 4.65 (1H, s), 5.12 (2H, s), 6.86 (2H, | | | | | | | | | |
| | d, J=8.7 Hz), 6.94-7.49 (10H, m). | | | | | | | | | |
| 124. | 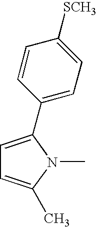 | 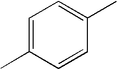 | 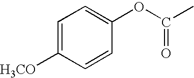 | COOH | O | 2 | 1 | 1 | 560 | 80 |
| | ¹H: 2.37 (3H, s), 2.50 (3H, s), 3.77 (3H, s), 3.95 (2H, t, J=6.09 Hz), 4.02 (2H, d, J=8.43 Hz), | | | | | | | | | |
| | 4.28 (2H, t, J=6.06 Hz), 4.53 (1H, s), 4.62 (1H, s), 5.96 (1H, d, J=3.12 Hz), 6.10 (1H, d, | | | | | | | | | |
| | J=3.36 Hz), 6.63-6.68 (2H, m), 6.84-6.87 (2H, dd, J=2.73 & 9.00 Hz), 7.00-7.06 (2H, m), | | | | | | | | | |
| | 7.15 (2H, d, J=7.22 Hz), 7.28-7.33 (4H, m). | | | | | | | | | |
| 125. | 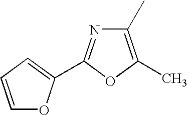 | 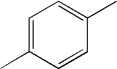 | 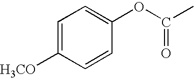 | COOH | O | 2 | 1 | 1 | 506 | 77 |
| | ¹H: 2.36 (3H, s), 2.97 (2H, t, J=5.15 Hz), 3.77 (3H, s), 4.01 (2H, s), 4.22 (2H, t, J=5.8 Hz), | | | | | | | | | |
| | 4.55 (1H, s), 4.63 (1H, s), 6.48-6.50 (1H, m), 6.83-6.88 (4H, m), 6.96 (1H, d, J=3.37 Hz), | | | | | | | | | |
| | 7.01-7.07 (2H, m), 7.21 (2H, d, J=8.04 Hz), 7.5 (1H, s). | | | | | | | | | |
| 126. | 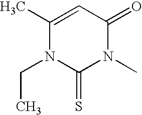 | 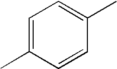 | 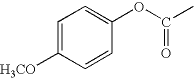 | COOH | O | 2 | 1 | 1 | 527 | 63 |
| | ¹H: 1.34 (3H, t, J=7.07 Hz)), 2.36 (3H, s), 3.5 (2H, t, J=6.07 Hz), 3.77 (3H, s), 4.04 (2H, d, | | | | | | | | | |
| | J=6.74 Hz), 4.26 (2H, t, J=6.94 Hz), 4.37 (2H, q, J=7.04 Hz), 4.56 (1H, s), 4.66 (1H, s), 6.23 | | | | | | | | | |
| | (1H, s), 6.87 (2H, dd, J=3.19 & 5.82 Hz), 6.94 (2H, t, J=7.6 Hz), 7.04 (2H, t, J=8.23 Hz), | | | | | | | | | |
| | 7.22-7.25 (2H, m). | | | | | | | | | |
| 127. | 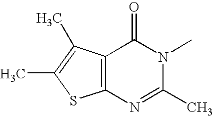 | 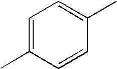 | 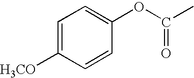 | COOH | O | 2 | 1 | 1 | 551 | 80 |
| | ¹H: 2.37 (3H, s), 2.39 (3H, s), 2.68 (3H, s), 3.77 (3H, s), 4.08 (2H, d, J=10.77 Hz), 4.36 (2H, | | | | | | | | | |
| | t, J=4.45 Hz), 4.58 (1H, s), 4.67 (1H, s), 4.83 (2H, t, J=4.47 Hz), 6.87 (2H, d, J=7.62 Hz), | | | | | | | | | |
| | 6.91-7.26 (6H, m) | | | | | | | | | |
| 128. | 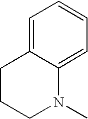 | 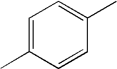 | 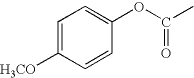 | COOH | O | 2 | 1 | 1 | 490 | 83 |
| | ¹H: 1.95 (2H, qui, J=5.94 Hz), 2.76 (2H, t, J=6.27 Hz), 3.43 (2H, t, J=5.49 Hz), 3.69 (2H, t, | | | | | | | | | |
| | J=5.67 Hz), 3.78 (3H, s), 4.03 (2H, t, J=5.28 Hz), 4.14 (2H, t, J=4.98 Hz), 4.56 (1H, s), 5.66 | | | | | | | | | |
| | (1H, s), 6.56-6.64 (2H, m), 6.84-6.89 (4H, m), 6.95 (1H, d, J=7.68 Hz), 7.01-7.07 (3H, m), | | | | | | | | | |
| | 7.22 (2H, d, J=8.22 Hz). | | | | | | | | | |
| 129. | 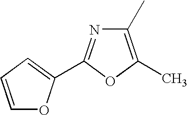 | 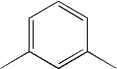 | 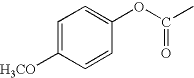 | COOH | O | 2 | 1 | 1 | 506 | 63 |
| | ¹H: 2.36 (3H, s), 2.96 (2H, t, J=7.37 Hz), 3.76 (3H, s), 4.07 (2H, s), 4.28 (2H, t, J=7.25 Hz), | | | | | | | | | |
| | 4.63 (1H, s), 4.69 (1H, s), 6.48-6.50 (1H, m), 6.81-6.85 (4H, m), 6.98-7.07 (4H, m), | | | | | | | | | |
| | 7.08-7.24 (1H, m), 7.50-7.51 (1H, m). | | | | | | | | | |
| 130. | 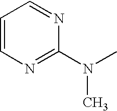 | 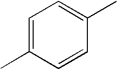 | 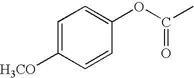 | COOH | O | 2 | 1 | 1 | 466 | 90 |
| | ¹H: 3.25 (3H, s), 3.78 (3H, s), 4.01-4.03 (4H, m), 4.21 (2H, t, J=5.5 Hz), 4.55 (1H, s), 4.65 | | | | | | | | | |
| | (1H, s), 6.49-6.52 (1H, m), 6.84-6.89 (4H, m), 7.01-7.07 (2H, m), 7.22 (2H, d, J=7.83 Hz), | | | | | | | | | |
| | 8.34 (2H, d, J=4.8 Hz). | | | | | | | | | |
| 131. | 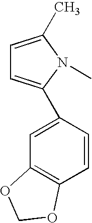 | 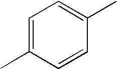 | 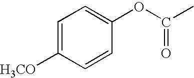 | COOH | O | 2 | 1 | 1 | 558 | 72 |
| | ¹H: 2.36 (3H, s), 3.77-3.83 (2H, m), 3.94-4.00 (2H, m), 4.07 (3H, s), 4.20-4.27 (2H, m), | | | | | | | | | |
| | 4.50-4.60 (2H, m), 5.92 (1H, d, J=3.00 Hz), 5.98 (2H, s), 6.03 (1H, d, J=3.30 Hz), 6.65-6.70 | | | | | | | | | |
| | (3H, m), 6.85-6.88 (4H, m), 7.02 (2H, d, J=8.98 Hz), 7.13-7.17 (2H, m) | | | | | | | | | |
| 132. | 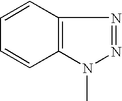 | 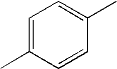 | 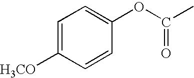 | COOH | O | 2 | 1 | 1 | 476 | 62 |
| | ¹H: 3.76 (3H, s), 4.08 (2H, s), 4.53 (2H, s), 4.64 (2H, t, J=5.59 Hz), 5.10 (2H, t, J=5.52 Hz), | | | | | | | | | |
| | 6.82-6.85 (4H, m), 7.00 (2H, d, J=8.80 Hz), 7.19 (2H, d, J=8.07 Hz), 7.37-7.41 (2H, m), | | | | | | | | | |
| | 7.86-7.89(2H, m). | | | | | | | | | |
| 133. | 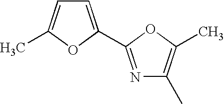 | 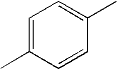 | 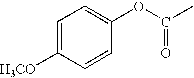 | COOH | O | 2 | 1 | 1 | 520 | 81 |
| | ¹H: 2.37 (3H, s), 2.42 (3H, s), 2.96 (2H, t, J=5.65 Hz), 4.02 (2H, s), 3.78 (3H, s), 4.20 (2H, t, | | | | | | | | | |
| | J=5.64 Hz), 4.55 (1H, s), 4.65 (1H, s), 6.08 (1H, d, J=2.63 Hz), 6.83-6.91 (5H, m), 7.01-7.07 | | | | | | | | | |
| | (2H, m), 7.20 (2H, d, J=8.30 Hz). | | | | | | | | | |
| 134. | 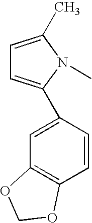 | 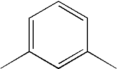 | 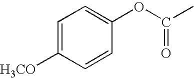 | COOH | O | 2 | 1 | 1 | 558 | 84 |
| | ¹H: 2.49 (3H, s), 3.72 (3H, s), 3.97 (2H, s), 4.20-4.22 (2H, m), 4.25-4.39 (2H, m), 4.55 (2H, | | | | | | | | | |
| | s), 5.80 (1H, d, J=3.18 Hz), 5.89 (1H, d, J=3.12 Hz), 6.02 (2H, s), 6.64-6.67 (2H, m), | | | | | | | | | |
| | 6.81-7.01 (8H, m), 7.17-7.20 (1H, m). | | | | | | | | | |
| 135. | 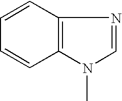 | 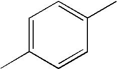 | 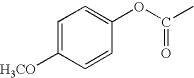 | COOH | O | 2 | 1 | 1 | 475 | 64 |
| | ¹H: 3.78 (3H, s), 4.04 (2H, s), 4.38-4.39 (2H, s), 4.53-4.56 (3H, m), 4.66 (1H, s), 6.62 (2H, d, | | | | | | | | | |
| | J=8.28 Hz), 6.85 (2H, d, J=8.94 Hz), 7.06 (2H, d, J=9.03 Hz), 7.18 (2H, d, J=8.55 Hz), | | | | | | | | | |
| | 7.32-7.39 (2H, m), 7.47 (1H, d, J=7.47 Hz), 7.80 (1H, d, J=7.8 Hz), 8.02 (1H, s). | | | | | | | | | |
| 136. | 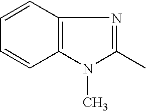 | 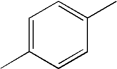 | 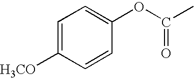 | COOH | O | 1 | 1 | 1 | 475 | 69 |
| | ¹H: 3.78 (3H, s), 3.93 (3H, s), 4.05 (2H, s), 4.60 (1H, s), 4.71 (1H, s), 5.51 (2H, s), 6.85 (2H, | | | | | | | | | |
| | d, J=7.38 Hz), 7.03-7.09 (4H, m), 7.27 (2H, m), 7.31-7.83 (4H, m) | | | | | | | | | |
| 137. | 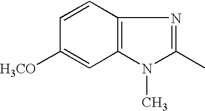 | 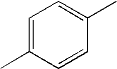 | 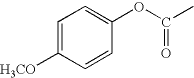 | COOH | O | 1 | 1 | 1 | 505 | 50 |
| | ¹H: 3.77 (3H, s), 3.87 (6H, s), 4.05 (2H, s), 4.60 (1H, s), 4.70 (1H, s), 5.42 (2H, s), 6.79-6.87 | | | | | | | | | |
| | (4H, m), 6.90-7.07 (4H, m), 7.26-7.69 (3H, m) | | | | | | | | | |
| 138. | 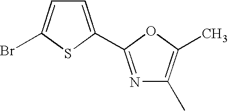 | 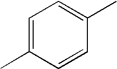 | 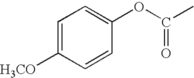 | COOH | O | 2 | 1 | 1 | 601 | 23 |
| | ¹H: 2.34 (3H, s), 2.94 (2H, t, J=5.95 Hz), 3.78 (3H, s), 4.03 (2H, s), 4.19 (2H, s), 4.56 (1H, | | | | | | | | | |
| | s), 4.65 (1H, s), 6.84-6.87 (4H, m), 7.03-7.07 (3H, m), 7.2 (2H, d, J=8.31 Hz), 7.32 (1H, m) | | | | | | | | | |
| 139. | 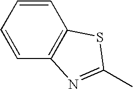 | 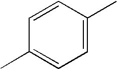 | 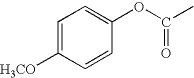 | COOH | O | 1 | 1 | 1 | 478 | 90 |
| | ¹H: 3.78 (3H, s), 4.04 (2H, s), 4.58 (2H, s), 5.5 (2H, s), 6.86 (2H, d, J=8.1 Hz), 7.0-7.03 (4H, | | | | | | | | | |
| | m), 7.26-7.29 (2H, m), 7.38-7.43 (1H, m), 7.48-7.53 (1H, m), 7.89 (1H, d, J=8.0 Hz), 8.03 | | | | | | | | | |
| | (1H, d, J=8.1 Hz) | | | | | | | | | |
| 140. | 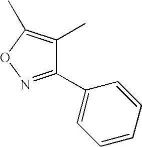 | 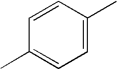 | 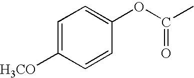 | COOH | O | 2 | 1 | 1 | 516 | 30 |
| | ¹H: 2.43 (3H, s), 2.89 (2H, m), 3.48 (2H, s), 3.63 (3H, s), 3.93 (2H, m), 4.35 (1H, s), 4.50 | | | | | | | | | |
| | (1H, s), 6.71 (2H, d, J=6.9 Hz), 6.84 (2H, d, J=7.92 Hz), 6.96 (2H, t, J=8.4 Hz), 7.10 (2H, t, | | | | | | | | | |
| | J=9.33 Hz), 7.49 (3H, s), 7.62 (2H, d, J=3.18 Hz) | | | | | | | | | |
| 141. | 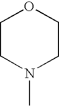 | 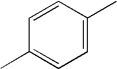 | 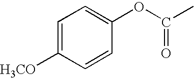 | COOH | O | 2 | 1 | 1 | 444 | 31 |
| | ¹H: 2.87 (4H, s), 3.02 (2H, m), 3.7 (3H, s), 3.80 (4H, s), 3.91 (2H, s), 4.18 (2H, m), 4.53 | | | | | | | | | |
| | (1H, s), 4.60 (1H, s), 6.80-6.86 (4H, m), 7.03 (2H, d, J=8.94 Hz), 7.19 (2H, d, J=8.31 Hz) | | | | | | | | | |
| 142. | 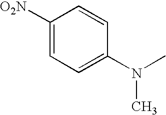 | 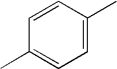 | 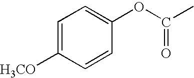 | COOH | O | 2 | 1 | 1 | 509 | 85 |
| | ¹H: 3.15 (3H, s), 3.70 (3H, s), 3.81-3.89 (4H, m), 4.08 (2H, t, J=5.49 Hz), 4.48 (1H, s), 4.52 | | | | | | | | | |
| | (1H, s), 6.63-6.68 (2H, m), 6.75 (4H, m), 6.94-7.0 (2H, m), 7.1-7.3 (2H, m), 8.06-8.11 (2H, | | | | | | | | | |
| | m) | | | | | | | | | |
| 143. | 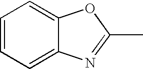 | 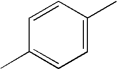 | 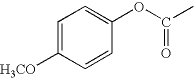 | COOH | O | 1 | 1 | 1 | 462 | 50 |
| | ¹H: 3.77 (3H, s), 3.98 (1H, s), 4.03 (1H, s), 4.53 (1H, s), 4.65 (1H, s), 5.39 (2H, s), 6.90 (2H | | | | | | | | | |
| | d, J=8.98 Hz), 7.0 (2H, d, J=8.94 Hz), 7.08 (2H, t, J=8.49 Hz), 7.29 (2H, d, J=8.91 Hz), | | | | | | | | | |
| | 7.39-7.43 (2H, m), 7.63 (1H, d, J=6.95 Hz), 7.7 (1H, d, J=6.95 Hz) | | | | | | | | | |
| 144. | 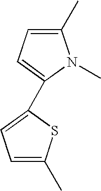 | 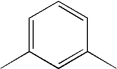 | 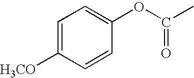 | COOH | O | 2 | 1 | 1 | 534 | 45 |
| | ¹H: 2.23 (3H, s), 2.39 (3H, s), 3.55 (3H, s), 3.77 (2H, s), 3.93 (2H, t, J=5.64 Hz), 4.21 (2H, | | | | | | | | | |
| | t, J=5.95 Hz), 4.36 (1H, s), 4.43 (1H, s), 5.85 (1H, d, J=2.94 Hz), 6.13 (1H, d, J=3.39 Hz), | | | | | | | | | |
| | 6.52-6.68 (6H, m), 6.73 (1H, d, J=3.33 Hz), 6.84 (2H, d, J=8.46 Hz), 6.91 (1H, d, J=3.57 | | | | | | | | | |
| | Hz). | | | | | | | | | |
| 145. | 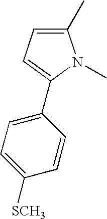 | 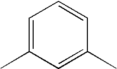 | 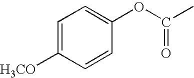 | COOH | O | 2 | 1 | 1 | 560 | 97 |

CD₃OD, ¹H: 2.3 (3H, s), 2.46 (3H, s), 3.77 (3H, s), 3.83 (2H, s), 3.96 (2H, t, J=5.6 Hz), 4.31
(2H, t, J=5.6 Hz), 4.5 (1H, s), 4.6 (1H, s), 5.85 (1H, d, J=3.3 Hz), 5.97 (1H, d, J=3.3 Hz), 6.6
(2H, m), 6.8 (3H, m), 7.0 (2H, m), 7.15 (1H, m), 7.25 (4H, m).

### Preparation 11

### L-Arginine salt of {Benzyl-[3-(3-phenothiazin-10-yl-propoxy)-benzyl]-amino}-acetic acid

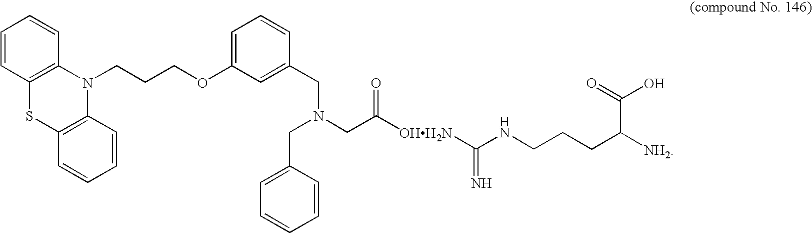

To a solution of {Benzyl-[3-(3-phenothiazin-10-yl-propoxy)-benzyl]-amino}-acetic acid. (compound No. 88) (100 mg) in ethanol (10 mL) heated to 60° C. was added another solution of L-arginine (34 mg) in water (0.2 mL) and the reaction mixture was heated to reflux for 3 hours. Solvent was evaporated under reduced pressure and the residue was triturated with ethyl acetate to obtain 80 mg of product. Preparation 12 Sodium and potassium salts of the compounds in table 3 were prepared by following the general procedure described below.

To a solution of carboxylic acid (mentioned in table 3) (1 mmol) in alcoholic solvent like methanol, ethanol and the like was added another solution of sodium or potassium alkoxides (0.95 mmol) in alcoholic solvent and the reaction mixture was stirred for 3 hours at 25-30° C. Solvent was evaporated and the residue was triturated with dry diethyl ether or diisopropyl ether to obtain the salt of the corresponding carboxylic acid. The compounds of the present invention lowered triglyceride, total cholesterol, LDL, VLDL and increased HDL and lowered serum glucose levels. This was demonstrated by in vivo animal experiments.

A) Demonstration of in vivo Efficacy of Compounds:

i) Serum triglyceride and total cholesterol lowering activity in Swiss albino mice: Male Swiss albino mice (SAM) were bred in Zydus animal house. All these animals were maintained under 12 hour light and dark cycle at 25±1° C. Animals were given standard laboratory chow (NIN, Hyderabad, India) and water ad libitum. SAM of 20-30 g body weight range was used. The protocol approved by Institutional Animal Ethics Committee is being used. The test compounds were administered orally to Swiss albino mice at 0.001 to 50 mg/kg/day dose for 6 days. The compound was administered after suspending it in 0.25% CMC or dissolving it in water, when compound is water-soluble. Control mice were treated with vehicle (0.25% of Carboxymethylcellulose; dose 10 ml/kg).

The blood samples were collected on oth day and in fed state 1 hour after drug administration on 6^{th} day of the treatment. The blood was collected in non heparinised capillary and the serum was analyzed for triglyceride and total cholesterol (Wieland, O. Methods of Enzymatic analysis. Bergermeyer, H., O., Ed., 1963. 211-214; Trinder, P. Ann. Clin Biochem. 1969. 6: 24-27). Measurement of serum triglyceride and total cholesterol was done using commercial kits (Zydus-Cadila, Pathline, Ahmedabad, India).

Formula for calculation:

Percentage reduction in triglycerides/total cholesterol were calculated according to the formula:

$$Percentage reduction ( \% ) = 1 - [ \frac{TT / OT}{TC / OC} ] ⨯ 100$$

OC=Zero day control group value OT=Zero day treated group value TC=Test day control group TT=Test day treated group

TABLE 1
Triglyceride lowering activity in Swiss albino mice:

| | Dose | % Triglyceride |
|---|---|---|
| Example No. | (mg/kg/day) | lowering |
| 113 | 10 | 43 |
| 122 | 10 | 35 |
| 130 | 3 | 38 |

ii) Serum Glucose Lowering Activity in db/db Mice Models

Homozygous animal C₅₇BL/KsJ-db/db mice are obese, hyperglycemic, hyperinsulinemic and insulin resistant (J. Clin. Invest., 85, 962-967, 1990), whereas heterozygous are lean and normoglycemic. The homozygous animals very closely mimic the human type II diabetes when blood sugar levels are not sufficiently controlled. Since this type of model resembles human type II diabetes meritus, the compounds of the invention were tested for their antidiabetic activity in this model. The compounds of the present invention showed serum glucose and triglycerides lowering activities. Male C₅₇ BL/KsJ-db/db mice of 8 to 14 weeks age, having body weight range of 40 to 60 grams, procured from the Jackson Laboratory, USA, were used in the experiment as per the protocol approved by the Institutional Animal Ethics Committee. Test compounds were suspended on 0.25%, carboxymethyl cellulose or dissolved in water when the compound is water soluble and administered to test group containing 6 animals at a dose of 0.001 mg to 50 mg/kg through oral gavage daily for 6 days. The control group received vehicle (dose 10 ml/kg). On the 6^{th} day, one hour after the drug dosing, blood was collected from retro-orbital sinus and the serum was analyzed for glucose and triglycerides were measured using commercial kits (Zydus-Cadila, Pathline, Ahmedabad, India). The serum glucose and triglyceride lowering activities of the test compound was calculated according of the formula:
Serum Glucose Lowering Activity (%)=

$$1 - [ \frac{TT / OT}{TC / OC} ] ⨯ 100$$

OC=Zero day control group value OT=Zero day treated group value TC=Test day control group TT=Test day treated group

| | Dose | Serum Glucose | Plasma TG |
|---|---|---|---|
| Example No. | (mg/kg/day) | reduction (%) | reduction (%) |
| 106 | 3 | 57 | — |

No adverse effects were observed for any of the mentioned compounds of invention. The compounds of the present invention showed good serum glucose, lipid and cholesterol lowering activity in the experimental animals used. These compounds are useful for the testing/prophylaxis of diseases caused by hyperlipidemia, hypercholesterolemia, hyperinsulinemia, hyperglycemia such as NIDM, cardiovascular diseases, stroke, hypertension, obesity since such diseases are interlinked to each other.

We claim:

1. A compound of formula (I) 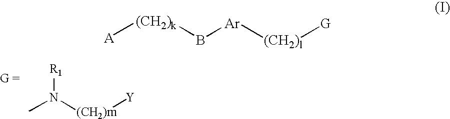
their tautomeric forms, their pharmaceutically acceptable salts, their. pharmaceutically acceptable solvates wherein
‘A’ represents an optionally substituted group selected from aryl, heteroaryl, heterocyclyl groups, each of them may optionally be fused; ‘B’ represents oxygen or sulfur; ‘Ar’ represents an optionally substituted divalent aromatic, heteroaromatic or a heterocyclic group, each of them may optionally be fused; RI represents hydrogen, optionally substituted groups selected from alkyl (linear or branched), alkenyl (linear or branched), alkynyl (linear or branched), aralkyl, aryloxycarbonyl, alkoxycarbonyl, alkynyloxycarbonyl, alkenyloxycarbonyl, arylcarbonyl, alkylcarbonyl, aryl, heteroaryl, heteroarylcarbonyl, alkylcarbonylamino, arylcarbonylamino, heteroarylcarbonylamino, alkoxycarbonylamino, aryloxycarbonylamino, heteroaryloxycarbonylamino, alkylsulfonyl, alkenylsulfonyl, alkynylsulfonyl, heteroaryloxycarbonyl, heterocyclyloxycarbonyl, alkylaminocarbonyl, arylaminocarbonyl, hydroxyalkyl, alkoxy, alkylsulfonyl, arylthiocarbonyl, heteroarylsulfonyl, arylsulfonyl groups; k, l and m are integers independently ranging from 1-3; Y is COR₃ (where R₃ is OH or substituted or unsubstituted alkoxy, aryloxy, aralkyloxy, NH₂, aminoalkyl, amiodialkyl, aminoaralkyl, aminoalkylaralyl groups); (CH₂)ₖ, (CH₂)ₗ, (CH₂)ₘ, may be optionally substituted with one or more substituents selected from optionally substituted alkyl, haloalkyl, aryl, alkenyl, alkoxy, aryloxy, aralkoxy, alkoxycarbonyl, aryloxycarbonyl and the like; with the proviso that, ‘A’ does not represent 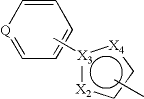
where Q is ‘C’ or ‘N’ and X₂, X₃ & X₄ are independently selected from C, N, O or S;

2. A compound as claimed in claim 1 wherein the substitutions on ‘A’ may be selected from hydroxyl, oxo, halo, thio, nitro, amino, cyano, formyl, alkyl, haloalkyl, perhaloalkyl, alkoxy, haloalkoxy, perhaloalkoxy, alkenyl, alkynyl, cycloalkyl, cycloalkenyl, bicycloalkyl, bicycloalkenyl, alkoxy, alkenoxy, cycloalkoxy, aryl, aryloxy, aralkyl, aralkoxy, heterocylyl, heteroaryl, heterocyclylalkyl, heteroaralkyl, heteroaryloxy, heteroaralkoxy, heterocyclyloxy, heterocyclylalkoxy, heterocyclylalkoxyacyl, acyl, acyloxy, acylamino, monosubstituted or disubstituted amino, arylamino, aralkylamino, carboxylic acid and its derivatives such as esters and amides, carbonylamino, hydroxyalkyl, aminoalkyl, alkoxyalkyl, aryloxyalkyl, aralkoxyalkyl, alkylthio, thioalkyl, arylthio, alkylsulfonylamino, alkylsulfonyloxy, alkoxycarbonylamino, aryloxycarbonylamino, aralkyloxycarbonylamino, aminocarbonylamino, alkylaminocarbonylamino, alkoxyamino, hydroxyl amino, sulfenyl derivatives, sulfonyl derivatives, sulfonic acid derivatives.

3. A compound as claimed in claim 1 wherein the substitutions on ‘Ar’ may be selected from optionally substituted linear or branched alkyl, alkoxy, thioalkyl, halogen, haloalkyl, haloalkoxy, acyl, amino, acylamino, thio or carboxylic acid derivatives or sulfonic acids or their derivatives.

4. A compound as claimed in claim 1, wherein the substituents on R₁ may be selected from from hydroxyl, oxo, halo, thio, nitro, amino, cyano, formyl, alkyl, haloalkyl, perhaloalkyl, alkoxy, haloalkoxy, perhaloalkoxy, alkenyl, alkynyl, cycloalkyl, cycloalkenyl, bicycloalkyl, bicycloalkenyl, alkoxy, alkenoxy, cycloalkoxy, aryl, aryloxy, aralkyl, aralkoxy, heterocylyl, heteroaryl, heterocyclylalkyl, heteroaralkyl, heteroaryloxy, heteroaralkoxy, heterocyclyloxy, heterocyclylalkoxy, heterocyclylalkoxyalkyl, heterocyclylalkoxyacyl, acyl, acyloxy, acylamino, monosubstituted or disubstituted amino, arylamino, aralkylamino, carboxylic acid and its derivatives such as esters and amides.

5. A compound as claimed in claim 1 selected from Ethyl-[4(2-phenoxazin- 10-yl-ethoxy)-benzylamino]-acetate; Ethyl-[4-(2-phenothiazin-10-yl-ethoxy)-benzylamino]-acetate; Methyl-[4-(2-oxo-3-phenyl-oxazolidin-5-ylmethoxy)-benzylamino]-acetate; Ethyl-[(6-benzyloxy-naphthalen-2-ylmethyl)-amino]-acetate; Ethyl-{[6:(1-phenyl-pentyloxy)-naphthalen-2-ylmethyl]-amino}-acetate; Ethyl-[4-(2-carbazol-9-yl-ethoxy)-benzylamino]-acetate; Ethyl-[4-(1-pyridin-2-yl-pyrrolidin-2-ylmethoxy)-benzylaminol-acetate; Ethyl-{4-[2-(2,3-dihydro-benzo[1,4]oxazin-4-yl)-ethoxy]-benzylamino}-acetate; Ethyl-(benzyl-{3-[2-(3,4-dihydro-2H-quiolino-1-yl)-ethoxy]-benzyl}-amino)-acetate; Ethyl-(benzyl-{3-[2-(4-methanesulfonyloxy-phenyl)-thoxy]-benzyl}-amino)- acetate; Ethyl-(benzyl-{3-[2-(4-hydroxy-phenyl)-ethoxy]-benzyl}-amino)-acetate; Ethyl-{benzyl-[3-(2-phenoxazin-10-yl-ethoxy)-benzyl]-amino}-acetate; Ethyl-{benzyl-[3-(2-carbazol-9-yl-ethoxy)-benzyl]-amino}-acetate; Ethyl-(benzyl-{3-[2-(5-ethyl-pyridin-2-yl)-ethoxy]-benzyl}-amino)-acetate; Ethyl-(benzyl-{3-[2-(2,3-dihydro-benzo[1,4]oxazin4-yl)-ethoxy]-benzyl}-amino)-acetate; Ethyl-(benzyl-{3-[2-(2,3-dihydro-benzo[1,4]thiazin-4-yl)-ethoxy]-benzyl}-amino)-acetic acid ethyl ester; Ethyl-{benzyl-[3-(2-indol-1-yl-ethoxy)-benzyl]-amino}-acetate; Ethyl-{benzyl-[3-(3-phenothiazin-10-yl-propoxy)-benzyl]-amino}-acetate; Ethyl-{benzyl-[3-(3-methyl-4-oxo-3,4-dihydro-quinazolin-2-ylmethoxy)-benzyl]-amino}-acetate; Ethyl-[benzyl-(3-{2-[2-methyl-5-(4-methylsulfanyl-phenyl)-pyrrol-1-yl]-ethoxy}-benzyl)- amino]-acetate; Ethyl-{benzyl-[3-(2-oxo-3-phenyl-oxazolidin-5-ylmethoxy)-benzyl]-amino}-acetate; Ethyl-((4-methoxy-phenoxycarbonyl)-[4-(2-phenoxazin- 10-yl-ethoxy)-benzyl]-amino}-acetate; Ethyl-{(4-methoxy-phenoxycarbonyl)-[4-(2-phenothiazin-10-yl-ethoxy)-benzyl]-amino}-acetate; Methyl-{(4-methoxy-phenoxycarbonyl)-[4-(2-oxo-3-phenyl-oxazolidin-5-ylmethoxy)-benzyl]-amino}-acetate; Ethyl-[(6-benzyloxy-naphthalen-2-ylmethyl)-(4-methoxy-phenoxycarbonyl)-amino]-acetate; Methyl-{(4-methoxy-phenoxycarbonyl)-[6-(l -phenyl-pentyloxy)-naphthalen-2-ylmethyl]-amino}-acetate; Ethyl-[{4-[2-(6,7-dihydro-4H-thieno[3,2-c]pyridin-5-yl)-ethoxy]-benzyl}-(4-methoxy-phenoxycarbonyl)-amino]-acetate; Ethyl-[{4-[2 (2,3-dihydro-benzo[1,4]thiazin4-yl)-ethoxy]-benzyl}-(4-methoxy-phenoxycarbonyl)-amino]- acetate; Ethyl-[[4-(2-indol-1-yl-ethoxy)-benzyl]-(4-methoxy-phenoxycarbonyl)-amino]- acetate; Ethyl-[[4-(2-carbazol-9-yl-ethoxy)-benzyl]-(4-methoxy-phenoxycarbonyl)-amino]-acetate; Ethyl- {(4-methoxy-phenoxycarbonyl)-[4-(1-pyridin-2-yl-pyrrolidin-2-ylmethoxy)-benzyl]- amino}-acetate; Ethyl-[{4-[2-(2,3-dihydro-benzo[1,4]oxazin-4-yl)-ethoxy]-benzyl}-(4-methoxy-phenoxycarbonyi)-amino]- acetate; Ethyl-(benzyl-{4-[2-(5-methyl-2-thi6phen-2-yl-oxazol-4-yl)-ethoxy]-benzyl}-amino)-acetate; Ethyl-(benzyl-{3-[2-(5-methyl-2-thiophen-2-yl-oxazol-4-yl)-ethoxy]-benzyl}-amino)-acetate; Ethyl-[benzyl-(4-{2-[2-methyl-5-(4-methylsulfanyl-phenyl)-pyrrol-1-yl]-ethoxy}-benzyl)- amino]-acetate; Ethyl-(benzyl-{4-[2-(methyl-pyridin-2-yl-amino)-ethoxy]-benzyl}-amino)-acetate; Ethyl-(benzyl-{4-[2-(5-ethyl-pyridin-2-yl)-ethoxy]-benzyl}-amino)-acetate; Ethyl-{benzyl-[4-(2-fluoro-benzyloxy)-benzyl]-amino}-acetate; Ethyl-[benzyl-(4-{2-[2-(4-methoxy-phenyl)-5-methyl-pyrrol-1-yl]-ethoxy}-benzyl)- amino]-acetate; Ethyl-[benzyl-(4-{2-[2-methyl-5-(5-methyl-thiophen-2-yl)-pyrrol-1-yl]-ethoxy}-benzyl)- amino]-acetate; Ethyl-[benzyl-(4-{2-[5-methyl-2-(5-methyl-thiophen-2-yl)-oxazol-4-yl]-ethoxy}-benzyl)- amino]-acetate; Ethyl-(benzyl-{4-[2-(2,3-dihydro-benzo[1,4]thiazin-4-yl)-ethoxy]-benzyl}-amino)-acetate; Ethyl-(benzyl-{4-[2-(5-methyl-2-thiophen-3-yl-oxazol-4-yl)-ethoxy]-benzyl}-amino)- acetate; Ethyl-({4-[2-(2-benzo[b]thiophen-2-yl-5-methyl-oxazol-4-yl)-ethoxy]-benzyl}-benzyl-amino)-acetate; Ethyl-fbenzyl-[4-(3-methyl-4-oxo-3,4-dihydro-quinazolin-2-ylmethoxy)-benzyl]-amino}-acetate; Ethyl-{benzyl-[4-(2-phenoxazin-10-yl-ethoxy)-benzyl]-amino}-acetate; Ethyl-(benzyl-{4-[2-(3,4-dihydro-2H-quinolin-1-yl)-ethoxy]-benzyl}-mino)-cetate; Ethyl-[{4-[2-(5-ethyl-pyridin-2-yl)-ethoxy]-benzyl}-(4-methoxy-henoxycarbonyl)-amino]-acetate; Ethyl-((4-methoxy-phenoxycarbo’ -(4-[2-(methyl-pyridin-2-yl-amino)-ethoxy]-benzyl}-amino)-acetate; Ethyl-[(4-methoxy-phenoxycarbonyl)-(4-{2-[2-methyl-5-(5-methyl-thiophen-2-yl)-pyrrol-1-yl]-ethoxy}-benzyl)-amino]-acetate; Methyl-((4-methoxy-phenoxycarbonyl)-(4-[2-(5-methyl-2-thiophen-2-yl-oxazol4-yl)-ethoxy]-benzyl}-amino)-acetate; Ethyl-[(4-methoxy-phenoxycarbonyl)-(4-(2-[5-methyl-2-(5-methyl-thiophen-2-yl)-oxazol-4-yl]-ethoxy}-benzyl)-amino]-acetate; Ethyl-{(4-methoxy-phenoxycarbonyl)-[4-(3-methyl-4-oxo-3,4-dihydro-quinazolin-2-ylmethoxy)-benzyl]-amino}-acetate; Ethyl-[[4-(2-fluoro-benzyloxy)-benzyl]-(4-methoxy-phenoxycarbonyl)-amino]-acetate; Ethyl-[(4-methoxy-phenoxycarbonyl)-(4-{2-[2-methyl-5-(4-methylsulfanyl-phenyl)-pyrrol-1-yl]-ethoxy}-benzyl)-amino]-acetate; Ethyl-[{4-[2-(2-furan-2-yl-5-methyl-oxazol-4-yl)-ethoxy]-benzyl}-(4-methoxy-phenoxycarbonyl)-amino]-acetate; Ethyl-[{4-[2-(3-ethyl-4-methyl-6-oxo-2-thioxo-3,6-dihydro-2H-pyrimidin-1-yl)-ethoxy]- benzyl}-(4-methoxy-phenoxycarbonyl)-amino]-acetate; Ethyl-((4-methoxy-phenoxycarbonyl)-(4-[2-(2,5,6-trimethyl-4-oxo4H-thieno[2,3-d]pyrimidin-3-yl)-ethoxy]-benzyl}-amino)-acetate; Ethyl-[{4-[2-(3,4-dihydro-2H-quinolin-1-yl)-ethoxy]-benzyl}-(4-methoxy-phenoxycarbonyl)-amino]-acetate; Ethyl-[{3-[2-(2-furan-2-yl-5-methyl-oxazol-4-yl)-ethoxy]-benzyl}-(4-methoxy-phenoxycarbonyl)-amino]-acetate; Ethyl-((4-methoxy-phenoxycarbonyl)-(4-[2-(methyl-pyrimidin-2-yl-amino)-ethoxy]- benzyl}-amino)-acetate; Ethyl-[{4-[2-(2-benzo[1,3]dioxol-5-yl-5-methyl-pyrrol-1-yl)-ethoxy]-benzyl}-(4methoxy-phenoxycarbonyl)-amino]-acetate; Ethyl-[[4-(2-benzoimidazol-1-yl-ethoxy)-benzyl]-(4-methoxy-phenoxycarbonyl)-amino]-acetate; Ethyl-{3-[2-(2-benzo[1,3]dioxol-5-yl-5-methyl-pyrrol-1-yl)-ethoxy]-benzyl}-(4-methoxy- phenoxycarbonyl)-amino]-acetate; Ethyl-[[4-(2-benzoimidazol-1-yl-ethoxy)-benzyl]-(4-methoxy-phenoxycarbonyl)-amino]-acetate; Ethyl-{(4-methoxy-phenoxycarbonyl)-[4-(1-methyl- 1H-benzoimidazol-2-ylmethoxy)-benzyl]-amino)-acetate; Ethyl-[(4-methoxy-phenoxycarbonyl)-(4-{2-[5-methyl-2-(5-methyl-furan-2-yl)-oxazol-4- yl]-ethoxy}-benzyl)-amino]-acetate; Ethyl- [[4-(6-methoxy-1-methyl-1H-benzoimidazol-2-ylmethoxy)-benzyl]-(4-methoxy- phenoxycarbonyl)-amino]-acetate; Ethyl-[(4-{2-[2-(5-bromo-thiophei-2-yl)-5-methyl-oxazol-4-yl]-ethoxy)-benzyl)-(4-methoxy-phenoxycarbonyl)-amino]-acetate; Ethyl-[[4-(benzothiazol-2-ylmethoxy)-benzyl]-(4-methoxy-phenoxycarbonyl)-amino]- acetate; Ethyl-[benzyloxycarbonyl-(4-{2-[2-methyl-5-(5-methyl-thiophen-2-yl)-pyrrol-1-yl]-ethoxy}-benzyl)-amino]- acetate; Ethyl-{(4-methoxy-phenoxycarbonyl)-[4-(2-morpholin-4-yl-ethoxy)-benzyl]-amino}-acetate; Ethyl-[(4-methoxy-phenoxycarbonyl)-(4-{2-[methyl-(4-nitro-phenyl)-aamino]-ethoxy}-benzyl)-amino]-acetate; Ethyl-[(4-methoxy-phenoxycarbonyl)-(3-{2-[2-methyl-5-(5-methyl-thiophen-2-yl)-pyrrol- 1-yl]ethoxy}benzyl)-amino]-acetate; Ethyl-[[4-(benzooxazol-2-ylmethoxy)-benzyl]-(4-methoxy-phenoxycarbonyl)-amino]- acetate; Ethyl-[(4-methoxy-phenoxycarbonyl)-(3-(2-[2-methyl-5-(4-methylsulfanyl-phenyl)- pyrrol-1-yl]-ethoxy}-benzyl)-amino]-acetate; Ethyl-((4-methoxy-phenoxycarbonyl)-{4-[2-(5-methyl-3-phenyI-isoxazol-4-yl)-ethoxy]-benzyl}-amino)-acetate; (Benzyl-{3-[2-(3,4-dihydro-2H-quinolin-1-yl)-ethoxy]-benzyl}-amino)-acetic acid and its pharmaceutically acceptable salts; (Benzyl-{3-[2-(4-methanesulfonyloxy-phenyl)-ethoxy]-benzyl}-amino)-acetic acid and its pharmaceutically acceptable salts; {Benzyl-[3-(2-phenoxazin-10-yl-ethoxy)-benzyl]-amino}-acetic acid and its pharmaceutically acceptable salts; {Benzyl-[3-(2-carbazol-9-yl-ethoxy)-benzyl]-amino}-acetic acid and its pharmaceutically acceptable salts; (Benzyl-{3-[2-(5-ethyl-pyridin-2-yl)-ethoxy]-benzyl}-amino)-acetic acid and its pharmaceutically acceptable salts; (Benzyl-{3-[2-(2,3-dihydro-benzo[1,4]oxazin-4-yl)-ethoxy]-benzyl}-amino)-acetic acid and its pharmaceutically acceptable salts; (Benzyl-{3-[2-(2,3-dihydro-benzo[1,4]thiazin-4-yl)-ethoxy)-benzyl}-amino)-acetic acid and its pharmaceutically acceptable salts; {Benzyl-[3-(2-indol-1-yl-ethoxy)-benzyl]-amino}-acetic acid and its pharmaceutically acceptable salts; {Benzyl-[3-(3-phenothiazin-10-yl-propoxy)-benzyl]-amino}-acetic acid and its pharmaceutically acceptable salts; {Benzyl-[3-(3-methyl-4-oxo-3,4-dihydro-quinazolin-2-ylmethoxy)-benzyl]-amino}-acetic acid and its pharmaceutically acceptable salts; [Benzyl-(3-{2-[2-methyl-5-(4-methylsulfanyl-phenyl)-pyrrol-1-yl]-ethoxy}-benzyl)- amino]-acetic acid and its pharmaceutically acceptable salts; {(4-Methoxy-phenoxycarbonyl)-[4-(2-phenoxazin-10-yl-ethoxy)-benzyl]-amino}-acetic acid and its pharmaceutically acceptable salts; {(4-Methoxy-phenoxycarbonyl)-[4-(2-phenothiazin-10-yl-ethoxy)-benzyl]-amino}-acetic acid and its pharmaceutically acceptable salts; {(4-Methoxy-phenoxycarbonyl)-[4-(2-oxo-3-phenyl-oxazolidin-5-ylmethoxy)-benzyl]-amino}-acetic acid and its pharmaceutically acceptable salts; [(6-Benzyloxy-naphthalen-2-ylmethyl)-(4-methoxy-phenoxycarbonyl)-amnino]-acetic acid and its pharmaceutically acceptable salts; {(4-Methoxy-phenoxycarbonyl)-[6-(1-phenyl-pentyloxy)-naphthalen-2-ylmethyl]-amino}- acetic acid and its pharmaceutically acceptable salts; [{4-[2-6,7-Dihydro4H-thieno[3,2-c]pyridin-5-yl)-ethoxy]-benzyl}-(4-methoxy- phenoxycarbonyl)-amino]-acetic acid and its pharmaceutically acceptable salts; [(4-[2-(2,3-Dihydro-benzo [1 ,4]thiazin4-yl)-ethoxy]-benzyl}-(4-methoxy-phenoxycarbonyl)-amino]-acetic acid and its pharmaceutically acceptable salts; [[4-(2-Indol-1-yl-ethoxy)-benzyl]-(4-methoxy-phenoxycarbonyl)-amino]-acetic acid and its pharmaceutically acceptable salts; [[4-(2-Carbazol-9-yl-ethoxy)-benzyl]-(4-methoxy-phenoxycarbonyl)-amino]-acetic acid and its pharmaceutically acceptable salts; {(4-Methoxy-phenoxycarbonyl)-[4-(1-pyridin-2-yl-pyrrolidin-2-ylmethoxy)-benzyl]-amino}-acetic acid and its pharmaceutically acceptable salts; [{4-[2-(2,3-Dihydro-benzo[1,4]oxazin4-yl)-ethoxy]-benzyl}-(4-methoxy-phenoxycarbonyl)-amino]-aceticacid and its pharmaceutically acceptable salts; (Carboxymethyl-{4-[2-(5-methyl-2-phenyl-oxazol-4-yl)-ethoxy]-benzyl}-amino)-acetic acid and its pharmaceutically acceptable salts; (Benzyl-{4-[2-(5-methyl-2-thiophen-2-yl-oxazol-4-yl)-ethoxy]-benzyl}-amino)-acetic acid and its pharmaceutically acceptable salts; (Benzyl-{3-[2-(5-methyl-2-thiophen-2-yl-oxazol-4-yl)-ethoxy]-benzyl}-amino)-acetic acid and its pharmaceutically acceptable salts; [Benzyl-(4-{2-[2-methyl-5-(4-methylsulfanyl-phenyl)-pyrrol- 1-yl]-ethoxy}-benzyl)-amino]-acetic acid and its pharmaceutically acceptable salts; (Benzyl-{4-[2-(methyl-pyridin-2-yl-amino)-ethoxy]-benzyl}-amino)-acetic acid and its pharmaceutically acceptable salts; (Benzyl-{4-[2-(5-ethyl-pyridin-2-yl)-ethoxy]-benzyl}-amino)-acetic acid and its pharmaceutically acceptable salts; {Benzyl-[4-(2-fluoro-benzyloxy)-benzyl]-amino}-acetic acid and its pharmaceutically acceptable salts; [Benzyl-(4-{2-[2-(4-methoxy-phenyl)-5-methyl-pyrrol-1-yl]-ethoxy)-benzyl)-amino]- acetic acid and its pharmaceutically acceptable salts; [Benzyl-(4- {2-[2-methyl-5-(5-methyl-thiophen-2-yl)-pyrrol- 1-yl]-ethoxy}benzyl)-amino’]- acetic acid and its pharmaceutically acceptable salts; [Benzyl-(4-{2-[5-methyl-2-(5-methyl-thiophen-2-yl)-oxazol-4-yl]-ethoxy}-benzyl)- amino]-acetic acid and its pharmaceutically acceptable salts; (13enzyl-{4-[2-(2,3-dihydro-benzo[1,4]thiazin-4-yl)-ethoxy]-benzyl)-amino)-acetic acid (Benzyl-{4-[2-(5-methyl-2-thiophen-3-yl-oxazol4-yl)-ethoxy]-benzyl}-amino)-acetic acid and its pharmaceutically acceptable salts; ({4-[2-(2-Benzo[b]thiophen-2-yl-5-methyl-oxazol-4-yl)-ethoxy]-benzyl}-benzyl-amino)-acetic acid and its pharmaceutically acceptable salts; (Benzyl-[4-(3-methyl4-oxo-3,4-dihydro-quinazolin-2-ylmethoxy)-benzyl]-amino}-acetic acid and its pharmaceutically acceptable salts; {Benzyl-[4-(2-phenoxazin-10-yl-ethoxy)-benzyl]-amino}-acetic acid and its pharmaceutically acceptable salts; (Benzyl-{4-[2-(3,4-dihydro-2H-quinolin-1-yl)-ethoxy]-benzyl}-amino)-acetic acid and its pharmaceutically acceptable salts; [{4-[2-(5-Ethyl-pyridin-2-yl)-ethoxy]-benzyl} -(4-methoxy-phenoxycarb6nyl)-amino]- acetic acid and its pharmaceutically acceptable salts; ((4-Methoxy-phenoxycarbonyl)- (4-[2-(methyl-pyridin-2-yl-amino)-ethoxy]-benzyl}- amino)-acetic acid and its pharmaceutically acceptable salts; [(4-Methoxy-phenoxycarbonyl)-(4--2-[2-methyl-5-(5-methyl-thiophen-2-yl)-pyrrol-1-yl]-ethoxy}-benzyl)-amino]-acetic acid and its pharmaceutically acceptable salts; ((4-Methoxy-phenoxycarbonyl)-{4-[2-(5-methyl-2-thiophen-2-yl-oxazol-4-yl)-ethoxy]-benzyl}-amino)-acetic acid and its pharmaceutically acceptable salts; [(4-Methoxy-phenoxycarbonyl)-(4-{2-[5-methyl-2-(5-methyl-thiophen-2-yl)-oxazol-4-yl]- ethoxy}-benzyl)-amino]-acetic acid and its pharmaceutically acceptable salts; {(4-Methoxy-phenoxycarbonyl)-[4-(3-methyl4-oxo-3,4-dihydro-quinazolin-2- ylmethoxy)-benzyl]-amino}-acetic acid and its pharmaceutically acceptable salts; [[4-(2-Fluoro-benzyloxy)-benzyl]-(4-methoxy-phenoxycarbonyl)-amino]-acetic acid [(4-Methoxy-phenoxycarbonyl)-(4-{2-[2-methyl-5-(4-methylsulfanyl-phenyl)-pyrrol-1-yl]-ethoxy}-benzyl)-amino]-acetic acid and its pharmaceutically acceptable salts; {4-[2-2-Furan-2-yl-5-methyl-oxazol-4-yl)-ethoxy]-benzyl}-(4-methoxy-phenoxycarbonyl)-amino]-acetic acid and its pharmaceutically acceptable salts; [{4-[2-(3-Ethyl4-methyl-6-oxo-2-thioxo-3,6-dihydro-2H-pyrimidin-1-yl)-ethoxy]- benzyl}-(4-methoxy-phenoxycarbonyl)-amino]-acetic acid and its pharmaceutically acceptable salts; (4-Methoxy-phenoxycarbonyl)-{4-[2-(2,5,6-t ethyl-4-oxo4H-thieno[2,3-d]pyrimidin- 3-yl)-ethoxy]-benzyl}-amino)-acetic acid and its’ pharmaceutically acceptable salts; [{4-[2-(3,4-Dihydro-2H-quinolin-1-yl)-ethoxy]-benzyl)-(4-methoxy-phenoxycarbonyl)- amino]-acetic acid and its pharmaceutically acceptable salts; [{3-[2-(2-Furan-2-yl-5-methyl-oxazol4-yl)-ethoxy]-benzyl}-(4-methoxy-phenoxycarbonyl)-amino]-acetic acid and its pharmaceutically acceptable salts; ((4-Methoxy-phenoxycarbonyl)-{4-[2-(methyl-pyrimidin-2-yl-amino)-ethoxy]-benzyl}-amino)-acetic acid and its pharmaceutically acceptable salts; [{4-[2-(2-Benzo[1,3]dioxol-5-yl-5-methyl-pyrrol-1-yl)-ethoxy]-benzyl}-(4-methoxy- phenoxycarbonyl)-amino]-acetic acid and its pharmaceutically acceptable salts; [[4-(2-Benzotriazol- 1-yl-ethoxy)-benzyl]-(4-methoxy-phenoxycarbonyl)-amino]-acetic acid and its pharmaceutically acceptable salts; [(4-Methoxy-phenoxycarbonyl)-(4-{2-[5-methyl-2-(5-methyl-furan-2-yl)-oxazol-4-yl]- ethoxy}-benzyl)-amino]-acetic acid and its pharmaceutically acceptable salts; [{3-[2-(2-Benzo[1,3]dioxol-5-yl-5-methyl-pyrrol- 1-yl)-ethoxy]-benzyl)-(4-methoxy- phenoxycarbonyl)-amino]-acetic acid and its pharmaceutically acceptable salts; [[4-(2-Benzoimidazol- 1-yl-ethoxy)-benzyl]-(4-methoxy-phenoxycarbonyl)-amino]-acetic acid and its pharmaceutically acceptable salts; {(4-Methoxy-phenoxycarbonyl)-[4-(1-methyl-1H -benzoimidazol-2-ylmethoxy)-benzyl]-amino}-acetic acid and its pharmaceutically acceptable salts; [[4(6-Methoxy-1-methyl-1H -benzoimidazol-2-ylmethoxy)-bepzyl]-(4-methoxy- phenoxycarbonyl)-amino]-acetic acid and its pharmaceutically acceptable salts; [(4-(2-[2-(5-Bromo-thiophen-2-yl)-5-methyl-oxazol-4-yl]-ethoxy}-benzyl)-(4-methoxy- phenoxycarbonyl)-amino]-acetic acid and its pharmaceutically acceptable salts; [(4-Methoxy-phenoxycarbonyl)-(3- {2-[2-methyl-5-(4-methylsulfanyl-phenyl)-pyrrol 1-yl]-ethoxy}-benzyl)-amino]-acetic acid and its pharmaceutically acceptable salts; [[4-(13enzothiazol-2-ylmethoxy)-benzyl]-(4-methoxy-phenoxycarbonyl)-amino]-acetic acid and its pharmaceutically acceptable salts; ((4-Methoxy-phenoxycarbonyl)-(4-[2-(5-methyl-3-phenyl-isoxazol4-yl)-ethoxy]-benzyl}-amino)-acetic acid and its pharmaceutically acceptable salts; {(4-Methoxy-phenoxycarbonyl)-[4-(2-morpholin4-yl-ethoxy)-benzyl]-amino}-acetic acid and its pharmaceutically acceptable salts; [(4-Methoxy-phenoxycarbonyl)-(4-{2-[methyl-(4-nitro-phenyl)-amino]-ethoxy}-benzyl)-amino]-acetic acid and its pharmaceutically acceptable salts; [(4-Methoxy-phenoxycarbonyl)-(3-{2-[2-methyl-5-(5-methyl-thiophen-2-yl)-pyrrol-1-yl]- ethoxy}-benzyl)-amino]-acetic acid and its pharmaceutically acceptable salts; [[4-(13enzooxazol-2-ylmethoxy)-benzyl]-(4-methoxy-phenoxycarbonyl)-amino]-acetic acid and its pharmaceutically acceptable salts;

6. A pharmaceutical composition which comprises compounds of formula (I), as claimed in any preceding claims and a pharmaceutically acceptable carrier, diluent, excipients or solvate.

7. A pharmaceutical composition according to claim 6, in the form of a tablet, capsule, powder, granule, syrup, solution or suspension.

8. A method of preventing or treating diseases caused by hyperlipidaemia, hypercholesteremia, hyperglycemia, obesity, impaired glucose tolerance, leptin resistance, insulin resistance, diabetic complications, comprising administering an effective, non-toxic amount of compound of formula (I) as defined in any preceding claims to a patient in need thereof.

9. The method according to any preceding claim, wherein the disease is type 2 diabetes, impaired glucose tolerance, dyslipidaemia, hypertension, obesity, atherosclerosis, hyperlipidaemia, coronary artery disease, cardiovascular disorders and other diseases wherein insulin resistance is the underlying pathophysiologal mechanism.

10. A method according to claim 8 or 9 which comprises administering a compound of formula (I), as defined in claims 1-5 and a pharmaceutically acceptable carrier, diluent, excipients or solvate to a patient in need thereof

11. Use of compounds of formula (I), their pharmaceutical compositions and medicines containing them as defined in any previous claims as a medicament suitable for the treatment of diseases mentioned in any of the aforesaid claims.

12. A process for preparing compound of formula (I) comprising
i) converting an aldehyde of formula (I) wherein all the symbols are as defined in claim 1, with a suitably protected amino acid of formula (III), wherein ‘Y’ denotes suitable protected carboxylic acid group to obtain the compound of formula (Ia), wherein all the symbols are as defined in claim 1, & R₁=H. 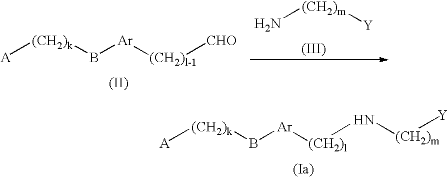
ii) converting the compounds of general formula (Ia) wherein all the symbols are as defined in claims 1, & R₁=H, to compounds of general formula (I) wherein all the symbols are as defined in claim 1, & R₃≢OH, by reacting with suitable aldehyde 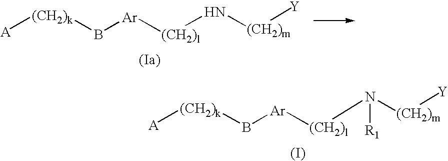
iii) deprotecting the compounds of general formula (I) to obtain the compounds of general formula (I) wherein R₃=OH & all other symbols are as defined in claim 1.

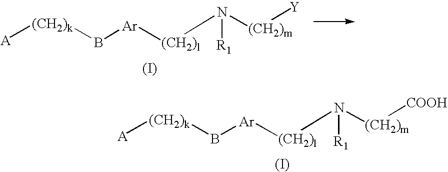

13. A process for preparing compound of formula (I) which comprises
i) reacting compounds of general formula (IV) where ‘L’ represents a suitable leaving group selected from halogen, mesylate, tosylate, tritlate, with compounds of general formula (V) where ‘Y’ represents suitably protected carboxyl group and all other symbols are as defined in claim 1, to obtain the compound of general formula (I), where R₃≢OH & NH₂; 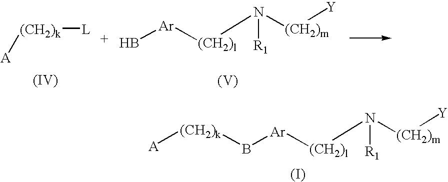
ii) deprotecting the compound of formula (I) to obtain a further compound of formula (I), wherein all symbols are as defined in claim 1 & R₃=OH. [chemical structure]
